SEQUENCE LISTING (20 sequences)

SEQ ID NO: 1 - protein, 1368 aa, Streptococcus pyogenes
  REGION 1..1368: note = MISC_FEATURE - AKP81606.1
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNSLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 2560 aa, Francisella tularensis
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNSLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEMSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 3 - protein, 15 aa, synthetic construct
  REGION 1..15: note = Synthetic
IGLDIGTNSVGWAVI

SEQ ID NO: 4 - protein, 8 aa, synthetic construct
  REGION 1..8: note = Synthetic Sequence
IVIEMARE

SEQ ID NO: 5 - protein, 27 aa, synthetic construct
  REGION 1..27: note = Synthetic Sequence
DVDHIVPQSFLKDDSIDNKVLTRSDKN

SEQ ID NO: 6 - protein, 8 aa, synthetic construct
  REGION 1..8: note = Synthetic Sequence
HHAHDAYL

SEQ ID NO: 7 - protein, 11 aa, synthetic construct
  REGION 1..11: note = Synthetic Sequence
YGRKKRRQRRR

SEQ ID NO: 8 - protein, 12 aa, synthetic construct
  REGION 1..12: note = Synthetic Sequence
RRQRRTSKLMKR

SEQ ID NO: 9 - protein, 27 aa, synthetic construct
  REGION 1..27: note = Synthetic Sequence
GWTLNSAGYLLGKINLKALAALAKKIL

SEQ ID NO: 10 - protein, 33 aa, synthetic construct
  REGION 1..33: note = Synthetic Sequence
KALAWEAKLAKALAKALAKHLAKALAKALKCEA

SEQ ID NO: 11 - protein, 16 aa, synthetic construct
  REGION 1..16: note = Synthetic Sequence
RQIKIWFQNRRMKWKK

SEQ ID NO: 12 - protein, 11 aa, synthetic construct
  REGION 1..11: note = Synthetic Sequence
YGRKKRRQRRR

SEQ ID NO: 13 - protein, 9 aa, synthetic construct
  REGION 1..9: note = Synthetic Sequence
RKKRRQRRR

SEQ ID NO: 14 - protein, 11 aa, synthetic construct
  REGION 1..11: note = Synthetic Sequence
YGRKKRRQRRR

SEQ ID NO: 15 - protein, 8 aa, synthetic construct
  REGION 1..8: note = Synthetic Sequence
RKKRRQRR

SEQ ID NO: 16 - protein, 11 aa, synthetic construct
  REGION 1..11: note = Synthetic Sequence
YARAAARQARA

SEQ ID NO: 17 - protein, 11 aa, synthetic construct
  REGION 1..11: note = Synthetic Sequence
THRLPRRRRRR

SEQ ID NO: 18 - protein, 11 aa, synthetic construct
  REGION 1..11: note = Synthetic Sequence
GGRRARRRRRR

SEQ ID NO: 19 - protein, 1307 aa, synthetic construct
  REGION 1..1307: note = Synthetic Sequence
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 20 - protein, 1228 aa, synthetic construct
  VARIANT 162: note = X can be any naturally occuring amino acid.
  VARIANT 188: note = X can be any naturally occuring amino acid.
  VARIANT 457: note = X can be any naturally occuring amino acid.
  VARIANT 532: note = X can be any naturally occuring amino acid.
  VARIANT 559: note = X can be any naturally occuring amino acid.
  VARIANT 593: note = X can be any naturally occuring amino acid.
  VARIANT 604: note = X can be any naturally occuring amino acid.
  VARIANT 627: note = X can be any naturally occuring amino acid.
  VARIANT 702: note = X can be any naturally occuring amino acid.
  VARIANT 723: note = X can be any naturally occurring amino acid.
  VARIANT 747: note = X can be any naturally occurring amino acid.
  VARIANT 950: note = X can be any naturally occurring amino acid.
  VARIANT 958: note = X can be any naturally occurring amino acid.
  VARIANT 987: note = X can be any naturally occurring amino acid.
  VARIANT 1036: note = X can be any naturally occurring amino acid.
  VARIANT 1129: note = X can be any naturally occurring amino acid.
  VARIANT 1132: note = X can be any naturally occurring amino acid.
  VARIANT 1135: note = X can be any naturally occurring amino acid.
  VARIANT 1138: note = X can be any naturally occurring amino acid.
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENXFSEEAKSTSIAFRCINENLTRYISNXDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIXKDLLDSVKSFENYIKAFFGEGKETNRDESFYGDFVLAYDILLKVDHIYDAIRNYVTQKPYSKDKFKLYFQNPQFXGGWDKDKETDYRATILRYGSKYYLAIXDKKYAKCLQKIDKDDVNGNYEKINYKLLPGPNKXLPKVFFSKKWXAYYNPSEDIQKIYKNGTFKKGDXFNLNDCHKLIDFFKDSISRYPKWSNAYDFNFSETEKYKDIAGFYREVEEQGYKVSFESASKKEVDKLVEEGKLYXFQIYNKDFSDKSHGTPNLHTXYFKLLFDENNHGQIRLSGGAELFXRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKXLIDKLNYXVDKKSNPCATGGALKGYQITNKFESFKSXSTQNGFIFYIPAWLTSKIDPSTGFVNLLKTKYTSIADSKKFISSFDRIXYVPEEDLFEFALDYKNFSRTDADYIKKWKLYSYGNRIRIFAAAKKNNVFAWEEVCLTSAYKELFNKYGINYQQGDIRALLCEQSDKAFYSSFXALXSLXLQXRNSITGRTDVDFLISPVKNSDGIFYDSRNYEAQENAILPKNADANGAYNIARKVLWAIGQFKKAEDEKLDKVKIAISNKEWLEYAQTSVK

(19) United States
(12) Patent Application Publication
(10) Pub. No.: US 2026/0132254 A1
(43) Pub. Date: May 14, 2026

(54) CATIONIC POLYMER AND USE FOR BIOMOLECULE DELIVERY

(71) Applicant: GenEdit Inc., Brisbane, CA (US)

(72) Inventors: Kunwoo Lee, Berkeley, CA (US); Santanu Maity, Berkely, CA (US)

(73) Assignee: GenEdit Inc., Brisbane, CA (US)

(21) Appl. No.: 19/239,086

(22) Filed: Jun. 16, 2025

Related U.S. Application Data
(62) Division of application No. 17/051,157, now Pat. No. 12,344,709.
(60) Provisional application No. 62/663,985, filed on Apr. 27, 2018.
(60) Provisional application No. 62/750,097, filed on Oct. 24, 2018.

(51) Int. Cl. C08G 69/10; A61K 9/51; A61K 47/10; A61K 47/64; A61K 47/69; C08G 69/04; C08G 69/48

(52) U.S. Cl. CPC C08G 69/10; A61K 9/5146; A61K 47/10; A61K 47/6455; A61K 47/6931; C08G 69/04; C08G 69/48

(57) ABSTRACT

Provided is a polymer comprising a structure of Formula 1:

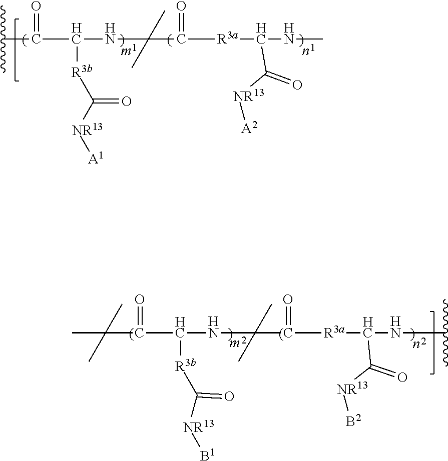

and a method of preparing said polymer. Also provided is a composition comprising the polymer and a nucleic acid and/or polypeptide, and a method of delivering a nucleic acid and/or polypeptide to a cell.

### CROSS-REFERENCE TO RELATED APPLICATIONS

This application claims the benefit of U.S. Provisional Patent Application No. 62/663,985 filed on Apr. 27, 2018, and U.S. Provisional Patent Application No. 62/750,097 filed on Oct. 24, 2018, the entire disclosures of which are hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: 39,273 Byte Extensible Markup Language (xml) file named 515539_ReplacementSequenceListing.xml, created on Aug. 6, 2025.

### BACKGROUND OF THE INVENTION

Peptide, protein, and nucleic based technologies have countless applications to prevent, cure and treat diseases. However, the safe and effective delivery of large molecules (e.g., polypeptides and nucleic acids) to their target tissues remains problematic. Accordingly, there continues to be a need for new compositions and methods useful for delivering therapeutic molecules.

### BRIEF SUMMARY OF THE INVENTION

Provided herein is a polymer comprising a structure of Formula 1:

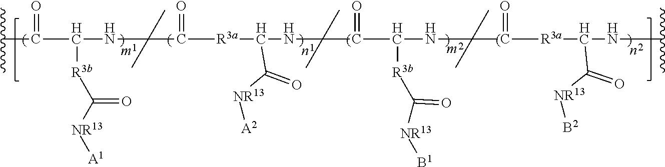

wherein:
each of m¹ and n¹ is an integer from 0 to 1000;
each of m² and n² is an integer from 0 to 1000, provided that the sum of m²+n² is greater than 5;
the symbol “/” indicates that the units separated thereby are linked randomly or in any order;
R^{3a} and R^{3b} are each independently a methylene or ethylene group;
each instance of R¹³ is independently hydrogen or a C₁-C₁₂ alkyl group, alkenyl group, cycloalkyl group, or cycloalkenyl group;
A¹ and A² are each independently a group of formula

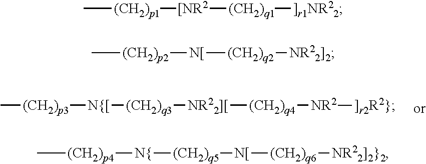

B1 and B2 are each independently

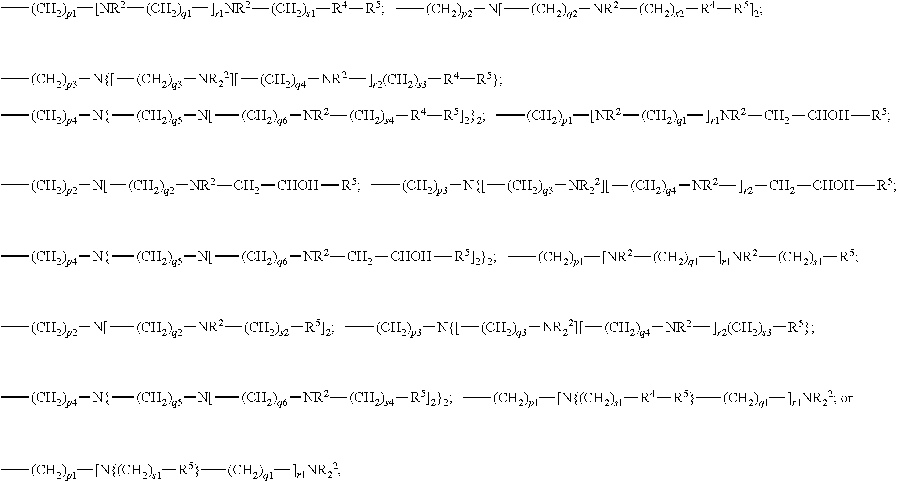

wherein p1 to p4, q1 to q6, r1 and r2, and s1 to s4 are each independently an integer of 1 to 5;
each instance of R² is independently hydrogen or a C₁-C₁₂ alkyl group, alkenyl group, cycloalkyl group, or cycloalkenyl group; each instance of R⁴ is independently —C(O)O—, —C(O)NH—, or —S(O)(O)—; and each instance of R⁵ is independently a substituted or unsubstituted alkyl group, cycloalkyl group, alkenyl group, cycloalkenyl group, aryl group, heteroalkyl group, heterocyclic group, or combination thereof optionally comprising from 1 to 8 (e.g., 2 to 8) secondary or tertiary amines or a substituent comprising a tissue-specific or cell-specific targeting moiety.

Also provided is a polymer of Formula 2:

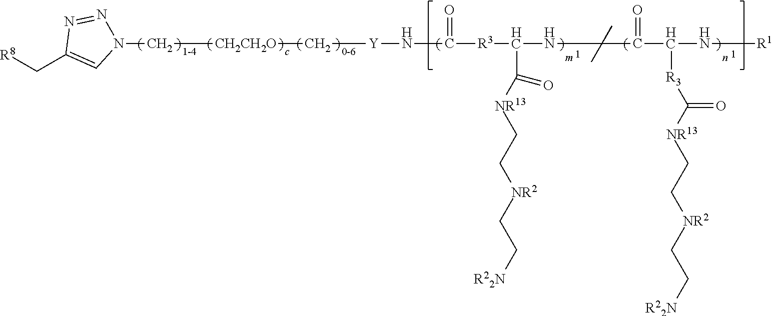

wherein:
each of m¹ and n¹ is an integer from 0 to 1000, provided that the sum of m¹ and n¹ is 10 to 2000;
c is an integer from 0 to 50;
Y is optionally present and is a cleavable linker;
R¹ is hydrogen, an aryl group, a heterocyclic group (e.g., aromatic or non-aromatic), a C₁-C₁₂ alkyl group, alkenyl group, cycloalkyl group, or cycloalkenyl group, or a C₁-C₁₂ linear or branched alkyl group optionally substituted with one or more substituents, such as one or more amines;
each instance of R² is independently hydrogen or a C₁-C₁₂ alkyl group, alkenyl group, cycloalkyl group, or cycloalkenyl group;
R⁸ is a is a tissue-specific or cell-specific targeting moiety; and
each instance of R¹³ is independently hydrogen or a C₁-C₁₂ alkyl group, alkenyl group, cycloalkyl group, or cycloalkenyl group.

The disclosure also provides a composition comprising a structure of Formula 1 and/or a polymer of Formula 2, and a nucleic acid and/or polypeptide. Further provided is a method of preparing a polymer comprising a structure of Formula 1 or a polymer of Formula 2, as well as methods of using the polymers and compositions comprising same, for example, to deliver a nucleic acid or protein to a cell.

### BRIEF DESCRIPTION OF THE DRAWINGS

FIG. 1 provides the amino acid sequence of Cas9 from Streptococcus pyogene (SEQ ID NO:1).

FIG. 2 provides the amino acid sequence of Cpf1 from Francisella tularensis subsp. Novicida U112 (SEQ ID NO:2).

FIG. 3 is a graph of the level of Cas9 RNP delivery as measured by green fluorescence protein (“GFP”) negative % in GFP-HEK cells at different dosages of polymer/Cas9 compositions.

FIG. 4 is a schematic illustration of an ai9 reporter allele.

FIG. 5 shows the level of Cre recombinase delivery to primary myoblasts as measured by the amount of RFP+ expression at different concentrations of polymer/Cre compositions.

FIG. 6 illustrates the level of Cre recombinase delivery in mouse muscle visualized by red fluorescence protein (lighter-colored portions indicate fluorescence).

FIG. 7 shows the indel mutation rate of human neuronal stem cells exhibited by Cas9 delivery.

FIG. 8 is a schematic illustration of a traffic light reporter in HEK 293T.

FIG. 9 shows the level of Cre recombinase delivery to TLR-HEK 293T as measured by the amount of RFP+ expression at different concentrations of polymer/Cre compositions.

FIG. 10 is a graph of the level of Cas9 RNP delivery as measured by green fluorescence protein (“GFP”) % in GFP-HEK cells at different dosages of polymer/Cas9 compositions.

FIG. 11 illustrates the level of eGFP mRNA delivery to HEK 293T cells as measured by GFP %.

FIG. 12 shows the incubation time dependence of the level of eGFP mRNA delivery to HEK 293T cells as measured by GFP %.

FIG. 13 illustrates level of red fluorescent protein (“RFP”) mRNA delivery to HEK 293T cells as measured by RFP+ expression in both the presence and absence of a 1.5 kDa PEG-PAsp(DET) polymer.

FIG. 14 illustrates level of red fluorescent protein (“RFP”) mRNA to HEK 293T cells as measured by RFP+ expression in both the presence and absence of a 1.5 kDa PEG-PAsp(DET) polymer.

FIG. 15 is a graph of the level of Cas9 RNP delivery as measured by green fluorescence protein (“GFP”) % in GFP-HEK cells for polymer/Cas9 nanoparticles in a buffer.

FIG. 16 provides the sequence of AsCpf1 (SEQ ID NO: 19).

FIG. 17 provides the sequence of LbCpf1 (SEQ ID NO: 20).

FIG. 18 is a graph showing the intensity averaged particle diameter (nm) of Polymers 2, 5, 33, 34, 35, and 36 as a function of time.

### DETAILED DESCRIPTION OF THE INVENTION

The invention provides a polymer comprising a structure of Formula 1:

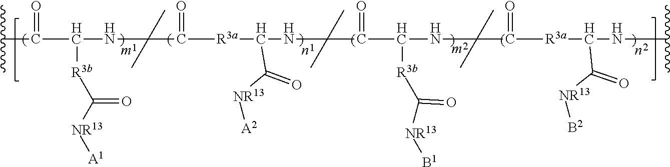

wherein:
each of m¹ and n¹ is an integer from 0 to 1000;
each of m² and n² is an integer from 0 to 1000, provided that the sum of m²+n² is greater than 5;
the symbol “/” indicates that the units separated thereby are linked randomly or in any order;
R^{3a} and R^{3b} are each independently a methylene or ethylene group;
each instance of R¹³ is independently hydrogen or a C₁-C₁₂ alkyl group, alkenyl group, cycloalkyl group, or cycloalkenyl group;
A¹ and A² are each independently a group of formula

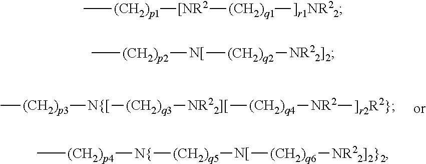

B1 and B2 are each independently

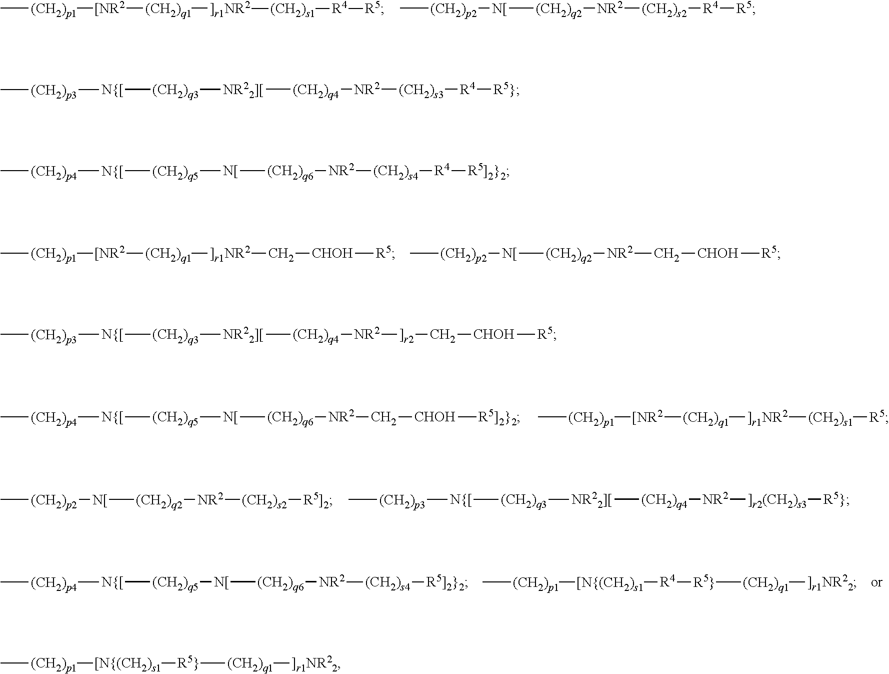

wherein
p1 to p4, q1 to q6, r1 and r2, and s1 to s4 are each independently an integer of 1 to 5;
each instance of R² is independently hydrogen or a C₁-C₁₂ alkyl group, alkenyl group, cycloalkyl group, or cycloalkenyl group;
each instance of R⁴ is independently —C(O)O—, —C(O)NH—, or —S(O)(O)—;
and each instance of R⁵ is independently a substituted or unsubstituted alkyl group, cycloalkyl group, alkenyl group, cycloalkenyl group, aryl group, heteroalkyl group, heterocyclic group, or combination thereof optionally comprising from 1 to 8 or 2 to 8 secondary or tertiary amines or a substituent comprising a tissue-specific or cell-specific targeting moiety.

As used herein, “alkyl” refers to a substituted or unsubstituted hydrocarbon chain. The alkyl group can have any number of carbon atoms (e.g., C₁-C₁₀₀ alkyl, C₁-C₅₀ alkyl, C₁-C₁₂ alkyl, C₁-C₈alkyl, C₁-C₆ alkyl, C₁-C₄ alkyl, C₁-C₂ alkyl, etc.). The hydrocarbon chain can be saturated, or in some embodiments can be unsaturated to any degree (e.g., providing alkenyl or alkynyl groups), and can be linear, branched, straight-chained, cyclic (e.g., cycloalkyl or cycloalkenyl), or a combination thereof. Cyclic groups can be monocyclic, fused to form bicyclic or tricyclic groups, linked by a bond, or spirocyclic. Cyclic groups comprise at least three carbons (e.g., C₃-C₁₂, C₃-C₁₀, C₃-C₈, C₃-C₆, or C₅-C₆). In some embodiments, the hydrocarbon chain can be interrupted by one or more heteroatoms (e.g., 1, 2, 3, 4, or 5 or more atoms of oxygen, nitrogen, and/or sulfur), thereby providing a heteroalkyl, heteroalkenyl, heteroalkynyl, or heterocyclyl (i.e., a heterocyclic group). In some embodiments, the alkyl, alkenyl, alkynyl is substituted with one or more substituents.

The term “heterocyclyl,” or “heterocyclic group” refers to a cyclic group, e.g., aromatic (e.g., heteroaryl) or non-aromatic where the cyclic group has one or more heteroatoms (e.g., 1, 2, 3, 4, or 5 or more atoms of oxygen, nitrogen, and/or sulfur). In some embodiments, the heterocyclyl or heterocyclic group (i.e., cyclic group, e.g., aromatic (e.g., heteroaryl) or non-aromatic where the cyclic group has one or more heteroatoms) is substituted with one or more substituents.

The term “aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. In some embodiments, the aryl group comprises an alkylene linking group so as to form an arylalkyl group (e.g., a benzyl group). Some aryl groups have from 6 to 12 ring members, such as phenyl, naphthyl or biphenyl. Other aryl groups have from 6 to 10 ring members, such as phenyl or naphthyl. In some embodiments, the aryl substituent comprise one or more heteroatoms (e.g., 1, 2, 3, 4, or 5 or more atoms of oxygen, nitrogen, and/or sulfur), thereby proving a heteroaryl group. In some embodiments, the aryl is substituted with one or more substituents.

It is understood that any of the foregoing groups may be univalent, bivalent, or multivalent (e.g., alkylene, alkenylene, alkynylene, arylene, heteroalkylene, heteroalkenylene, heteroalkynylene, heterocyclylene, heteroarylene, etc.).

As used herein, the term “substituted” can mean that one or more hydrogens on the designated atom or group (e.g., substituted alkyl group) are replaced with another group provided that the designated atom's normal valence is not exceeded. For example, when the substituent is oxo (i.e., ═O), then two hydrogens on the atom are replaced. Substituent groups can include one or more of an aldehyde, an ester, an amide, a ketone, nitro, cyano, fluoroalkyl (e.g., trifluoromethane), halo (e.g., fluoro), aryl (e.g., phenyl), heterocyclyl or heterocyclic group (i.e., cyclic group, e.g., aromatic (e.g., heteroaryl) or non-aromatic where the cyclic group has one or more heteroatoms), oxo, or combinations thereof. Combinations of substituents and/or variables are permissible provided that the substitutions do not significantly adversely affect synthesis or use of the compound.

According to Formula 1, each of m¹ and n¹ is an integer from 0 to 1000, such as 0-500, 0-200, 0-100, or 0-50. Furthermore, each of m² and n² is an integer from 0 to 1000, such as 0-500, or 0-100, provided that the sum of m²+n² is greater than 5 (e.g., 5-2000, 5-1000, 5-500, 5-200, 5-100, or 5-50). In other words, the polymer comprises at least some monomeric units of comprising groups B¹ and/or B², herein referred to collectively as the “B monomers.” In some embodiments, m¹ and n¹ are zero, such that the polymer comprises no A¹ or A² groups. In other embodiments, the polymer comprises some A¹ and/or A² groups and some B¹ and/or B² groups. In such embodiments, the ratio of (m¹+n¹)/(m²+n²) is about 20 or less (e.g., about 10 or less, about 5 or less, about 2 or less, or even about 1 or less). Also, in some embodiments, the ratio of (m¹+n¹)/(m²+n²) is about 0.2 or more, such as about 0.5 or more.

The polymer can exist as any suitable structure type. For example, the polymer can exist as an alternating polymer, random polymer, block polymer, graft polymer, linear polymer, branched polymer, cyclic polymer, or a combination thereof. In certain embodiments, the polymer is a random polymer, block polymer, graft polymer, or a combination thereof.

Thus, in the structure of Formula 1, the monomers (which can be referred to by their respective side chains A¹, A², B¹, and B²) can be arranged randomly or in any order. The integers m¹, n¹, m², and n² merely denote the number of the respective monomers that appear in the chain overall, and do not necessarily represent blocks of those monomers, although blocks or stretches of a given monomer might be present in some embodiments. For instance, the structure of Formula 1 can comprise the monomers in the order -A¹-A²-B¹-B²-, -A²-A¹-B²-B¹-, -A¹-B¹-A²-B²-, etc. In some embodiments, the polymer has a polypeptide (e.g., polyaspartamide) backbone arranged in an alpha/beta configuration, such that the “1” and “2” monomers alternate (e.g., -A¹-A²-B¹-B²-, -A²-A¹-B²-B¹-, -A¹-B²-B¹-A²-, -A²-B¹-B²-A¹-, -B¹-A²-B¹-A²-, etc.). However, the “A” and “B” monomers or sidechains (e.g., A¹/A² and B¹/B²) can be dispersed randomly throughout the polymer backbone, or can be arranged in blocks, or some combination thereof.

By way of further illustration, the polymer could instead be described as Formula 1.1:

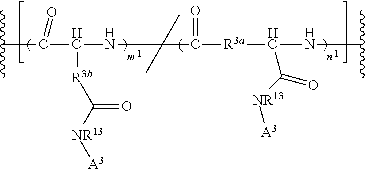

or, where the backbone is in an alpha, beta configuration, as Formula 1.2:

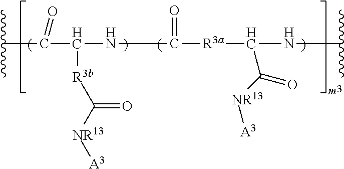

wherein,
m¹ and n¹ are as previously described;
m³ is an integer from 5-2000 (e.g., 5-1000, 5-500, 5-100, 25-2000, 25-500, 25-100, 50-2000, 50-1000, 50-500, or 50-100);
the symbol “/” indicates that the units separated thereby are linked randomly or in any order;
R^{3a} and R^{3b} are each independently a methylene or ethylene group;
each instance of R¹³ is independently hydrogen or a C₁-C₁₂ alkyl group, alkenyl group, cycloalkyl group, or cycloalkenyl group;
each A³ is independently a group of formula

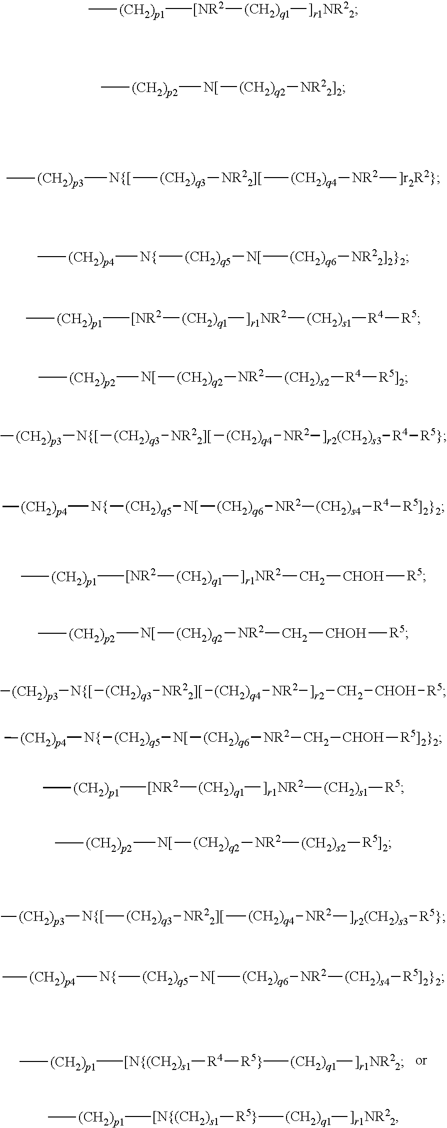

wherein p1 to p4, q1 to q6, r1 and r2, and s1 to s4 are each independently an integer of 1 to 5;
each instance of R² is independently hydrogen or a C₁-C₁₂ alkyl group, alkenyl group, cycloalkyl group, or cycloalkenyl group;
each instance of R⁴ is independently —C(O)O—, —C(O)NH—, or —S(O)(O)—; and
each instance of R⁵ is independently an alkyl group, cycloalkyl group, alkenyl group, cycloalkenyl group, aryl group, heteroalkyl group, heterocyclic group, or combination thereof optionally comprising from 1 to 8 or 2 to 8 secondary or tertiary amines or a substituent comprising a tissue-specific or cell-specific targeting moiety;
provided that at least about 5% (such as at least about 10%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or even 100%) of the A³ groups are selected from

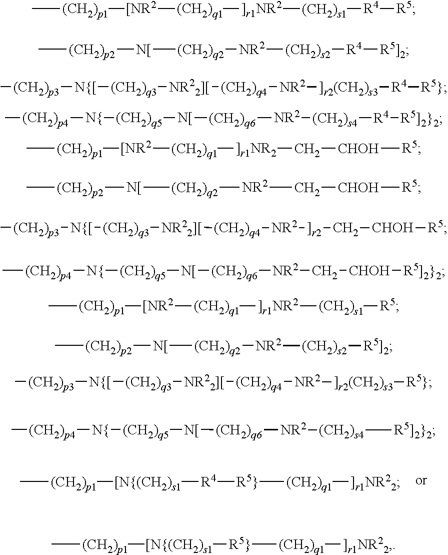

In still other embodiments, the polymer has some or even a majority of A³ groups (at least about 5%, at least about 10%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%) that are selected from

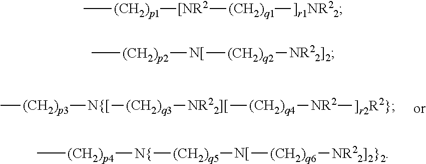

In any of the forgoing polymer structures, R^{3a} and R^{3b} are each independently a methylene or ethylene group. In some embodiments, R^{3a} is an ethylene group and R^{3b} is a methylene group; or R^{3a} is a methylene group and R^{3b} is an ethylene group. In certain embodiments, R^{3a} and R^{3b} are each an ethylene group. In preferred embodiments, R^{3a} and R^{3b} are each a methylene group.

Groups A¹ and A² are independently selected and, therefore, can be the same or different from one another. Similarly, groups B¹ and B² are independently selected and can be the same or different from one another. Furthermore, each instance of A³ can be the same or different from other A³ groups.

In groups A¹, A², A³, B¹, and B², integers p1 to p4 (i.e., p1, p2, p3, and p4), q1 to q6 (i.e., q1, q2, q3, q4, q5, and q6), and r1 and r2 are each independently an integer of 1 to 5 (e.g., 1, 2, 3, 4, or 5). In some embodiments, p1 to p4 (i.e., p1, p2, p3, and p4), q1 to q6 (i.e., q1, q2, q3, q4, q5, and q6), and/or r1 and r2 are each independently an integer of 1 to 3 (e.g., 1, 2, or 3). In certain embodiments, p1 to p4 (i.e., p1, p2, p3, and p4) and/or q1 to q6 (i.e., q1, q2, q3, q4, q5, and q6) are each 2. In some embodiments, p1 to p4 (i.e., p1, p2, p3, and p4) and/or q1 to q6 (i.e., q1, q2, q3, q4, q5, and q6) are each 2, and r1 and r2 are each 1. In some embodiments, s1-s4 are each independently an integer of 1, 2, 3, 4, or 5, such as 1, 2, or 3, or 1-2.

In the polymer structures, each instance of R⁴ is independently —C(O)O—, —C(O)NH—, or —S(O)(O)—. In some embodiments, each instance of R⁴ is independently —C(O)O— or —C(O)NH—. In certain embodiments, each instance of R⁴ is —C(O)O—. In certain embodiments, each instance of R⁴ is —C(O)NH—.

In some embodiments of Formula 1, each of A¹ and A² is a group of formula —(CH₂)ₚ₁—[NH—(CH₂)_{q1}—]ᵣ₁NH₂, such as a group —(CH₂)₂—NH—(CH₂)₂—NH₂. In addition, or alternatively, each of B¹ and B² is a group of formula —(CH₂)ₚ₁—[NH—(CH₂)_{q1}—]ᵣ₁NH—(CH₂)₂—R⁴—R⁵, such as a group —(CH₂)₂—NH—(CH₂)₂—NH—(CH₂)₂—R⁴—R⁵, or a group —(CH₂)₂—NH—(CH₂)₂—NH—(CH₂)₂—C(O)—O—R⁵. In some embodiments, each of B¹ and B² is a group of the formula —(CH₂)ₚ₁—[NR²—(CH₂)_{q1}—]ᵣ₁NR²—(CH₂)ₛ₁—R⁵ or —(CH₂)₂—NR² (CH₂)₂—NR²—(CH₂)₂—R⁵, optionally wherein each R² is independently a C₁-C₃ alkyl (e.g., methyl), and optionally wherein R⁵ is a C₁-C₃ alkyl amino or C₁-C₃ dialkylamino (e.g., R⁵ is —CH₂—NH—CH₃ or —CH₂—N—(CH₃)₂).

In some embodiments of Formula 1.1, A³ is a group of the formula —(CH₂)ₚ₁—[NH—(CH₂)_{q1}—]ᵣ₁NH₂, such as a group —(CH₂)₂—NH—(CH₂)₂—NH₂; or a group of formula —(CH₂)ₚ₁—[NH—(CH₂)_{q1}—]ᵣ₁NH—(CH₂)₂—R⁴—R⁵, such as a group —(CH₂)₂—NH—(CH₂)₂—NH—(CH₂)₂—R⁴—R⁵: —(CH₂)₂—NH—(CH₂)₂—NH—(CH₂)₂—C(O)—O—R⁵; or a group —(CH₂)ₚ₁—[NR²—(CH₂)_{q1}—]ᵣ₁NR²—(CH₂)ₛ₁—R⁵, such as —(CH₂)₂—NR²—(CH₂)₂—NR²—(CH₂)₂—R⁵, optionally wherein each R² is independently a C₁-C₃ alkyl (e.g., methyl), and optionally wherein R⁵ is a C₁-C₃ alkyl amino or C₁-C₃ dialkylamino (e.g., R⁵ is —CH₂—NH—CH₃ or —CH₂—N—(CH₃)₂); provided that at least about 5% (such as at least about 10%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or even 100%) of the A³ groups are —(CH₂)ₚ₁—[NH—(CH₂)_{q1}—]ᵣ₁NH—(CH₂)₂—R⁴—R⁵, such as a group —(CH₂)₂—NH—(CH₂)₂—NH—(CH₂)₂—R⁴—R⁵, or a group —(CH₂)₂—NH—(CH₂)₂—NH—(CH₂)₂—C(O)—O— R⁵; or a group —(CH₂)ₚ₁—[NR²—(CH₂)_{q1}—]ᵣ₁NR²—(CH₂)ₛ₁—R⁵, such as —(CH₂)₂—NR² (CH₂)₂—NR²—(CH₂)₂—R⁵, optionally wherein each R² is independently a C₁-C₃ alkyl (e.g., methyl), and optionally wherein R⁵ is a C₁-C₃ alkyl amino or C₁-C₃ dialkylamino (e.g., R⁵ is —CH₂—NH—CH₃ or —CH₂—N—(CH₃)₂). Additionally, in some embodiments, the polymer has some or even a majority of A³ groups (at least about 5%, at least about 10%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%) that are —(CH₂)ₚ₁—[NH—(CH₂)_{q1}—]ᵣ₁NH—(CH₂)₂—R⁴—R⁵, such as a group —(CH₂)₂—NH—(CH₂)₂—NH—(CH₂)₂—R⁴—R⁵, or a group —(CH₂)₂—NH—(CH₂)₂—NH—(CH₂)₂—C(O)—O—R⁵; or a group —(CH₂)ₚ₁—[NR²—(CH₂)_{q1}—]ᵣ₁NR²—(CH₂)ₛ₁—R⁵, such as —(CH₂)₂—NR²—(CH₂)₂—NR²—(CH₂)₂—R⁵, optionally wherein each R² is independently a C₁-C₃ alkyl (e.g., methyl), and optionally wherein R⁵ is a C₁-C₃ alkyl amino or C₁-C₃ dialkylamino (e.g., R⁵ is —CH₂—NH—CH₃ or —CH₂—N—(CH₃)₂).

Furthermore, in the polymer structures, each instance of R⁵ is independently an alkyl group, cycloalkyl group, alkenyl group, cycloalkenyl group, aryl group, heteroalkyl group, heterocyclic group, or combination thereof, optionally comprising from 1 to 8 (or 2 to 8) secondary or tertiary amines. Any of the foregoing groups of R⁵ can be substituted or unsubstituted. R⁵ also can be a substituent comprising a tissue-specific or cell-specific targeting moiety. In some embodiments, R⁵ can comprise from about 1 to about 50 carbon atoms (e.g., from about 2 to about 50 carbon atoms, such as about 2 to about 40 carbon atoms, from about 2 to about 30 carbon atoms, from about 2 to about 20 carbon atoms, from about 1 to about 16 or about 2 to about 16 carbon atoms, from about 1 to about 12 or about 2 to about 12 carbon atoms, from about 1 to about 10 or about 2 to about 10 carbon atoms, or from about 1 to about 8 or about 2 to about 8 carbon atoms). In some embodiments, R⁵ is a heteroalkyl group comprising from 1 to 8 or 2 to 8 (i.e., 1, 2, 3, 4, 5, 6, 7, or 8) secondary or tertiary amines. The secondary or tertiary amines can be part of the heteroalkyl backbone (i.e., the longest continuous chain of atoms in the heteroalkyl group, or a pendant substituent. Thus, for instance, the heteroalkyl group comprising the secondary or tertiary amines can be an alkylamino group, dialkylamino group, amino alkyl group, alkylaminoalkyl group, dialkylaminoalkyl group, aminoalkylamino group, or the like comprising 1 to 8 (or 2-8) secondary or tertiary amines.

In some embodiments, each R⁵ is independently selected from:

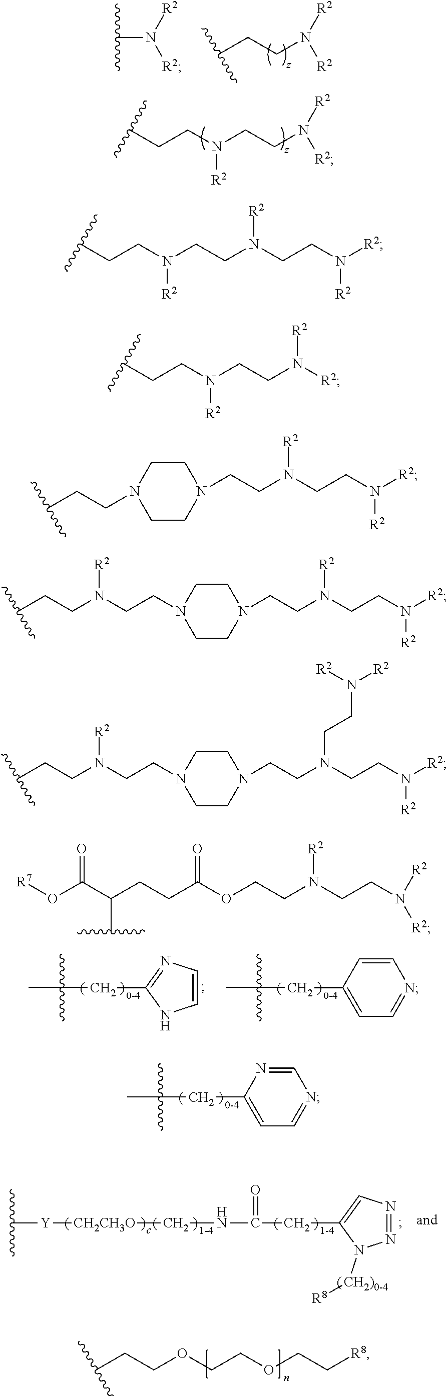

wherein
each instance of R² is independently hydrogen or a C₁-C₁₂ alkyl group, alkenyl group, cycloalkyl group, or cycloalkenyl group;
R⁷ is a C₁-C₅₀ alkyl group, alkenyl group, cycloalkyl group, or cycloalkenyl group optionally substituted with one or more amines;
z is an integer from 1 to 5;
c is an integer from 0 to 50;
Y is optionally present and is a cleavable linker;
n is an integer from 0 to 50; and
R⁸ is a tissue-specific or cell-specific targeting moiety, C₁-C₁₂ alkyl group, alkenyl group, cycloalkyl group, or cycloalkenyl group

Each instance of R² can be hydrogen or a C₁-C₁₂ alkyl group, alkenyl group, cycloalkyl group, or cycloalkenyl group. In some embodiments, R² is a C₁-C₁₂ (e.g., a C₁-C₁₀ alkyl group; a C₁-C₈alkyl group; a C₁-C₆ alkyl group; a C₁-C₄ alkyl group, a C₁-C₃ alkyl group, or a C₁ or C₂ alkyl group) linear or branched alkyl group. In certain embodiments, R² is methyl or hydrogen.

R⁷ can be a C₁-C₅₀ (e.g., C₁-C₄₀, C₁-C₃₀, C₁-C₂₀, C₁-C₁₀, C₄-C₁₂, or C₆-C₈) alkyl group, alkenyl group, cycloalkyl group, or cycloalkenyl group optionally substituted with one or more amines. In some embodiments, R⁷ is a C₄-C₁₂, such as a C₆-C₈, alkyl group, alkenyl group, cycloalkyl group, or cycloalkenyl group optionally substituted with one or more amines. In some embodiments, R⁷ is substituted with one or more amines. In certain embodiments, R⁷ is substituted with 1 to 8 or 2 to 8 (i.e., 1, 2, 3, 4, 5, 6, 7, or 8) secondary or tertiary amines. The amines can be a part of the alkyl group (i.e., encompassed in the alkyl group backbone) or a pendant substituent.

R¹³ can be hydrogen or a C₁-C₁₂ alkyl group, alkenyl group, cycloalkyl group, or cycloalkenyl group. In some embodiments, R¹³ is a C₁-C₁₂ (e.g., a C₁-C₁₀ alkyl group; a C₁-C₈ alkyl group; a C₁-C₆ alkyl group; a C₁-C₄ alkyl group, a C₁-C₃ alkyl group, or a C₁ or C₂ alkyl group) linear or branched alkyl group. In certain embodiments, R¹³ is methyl. In other embodiments, R¹³ can be hydrogen. Typically, within a given polymer, each R¹³ is the same (e.g., all methyl or all hydrogen); however, each R¹³ is independently chosen and can be the same or different.

Each instance of Y is optionally present. As used herein, the phrase “optionally present” means that a substituent designated as optionally present can be present or not present, and when that substituent is not present, the adjoining substituents are bound directly to each other. When Y is present, Y is a cleavable linker. As used herein, the phrase “cleavable linker” refers to any chemical element that connects two species that can be cleaved as to separate the two species. For example, the cleavable linker can be cleaved by a hydrolytic process, photochemical process, radical process, enzymatic process, electrochemical process, or a combination thereof. Exemplary cleavable linkers include, but are not limited to:

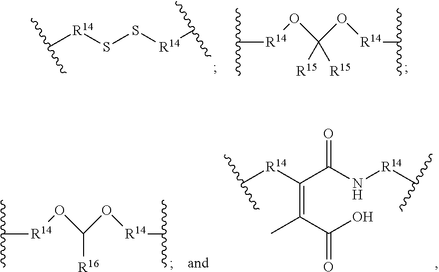

wherein each occasion of R¹⁴ independently is a C₁-C₄ alkyl group, each occasion of R¹⁵ independently is hydrogen, an aryl group, a heterocyclic group (e.g., aromatic or non-aromatic), a C₁-C₁₂ alkyl group, alkenyl group, cycloalkyl group, or cycloalkenyl group, and R¹⁶ is a six-membered aromatic or heteroaromatic group optionally substituted with one or more —OCH₃, —NHCH₃, —N(CH₃)₂, —SCH₃, —OH, or a combination thereof.

The polymer can be any suitable polymer, provided the polymer comprises the foregoing polymer structure as part of the polymer. In some embodiments, the polymer is a block copolymer comprising a polymer block having the structure of Formula 1, 1.1, or 1.2, and one or more other polymer blocks (e.g., an ethylene oxide subunit, or a propylene oxide subunit). In other embodiments, the structure of Formula 1, 1.1, or 1.2 is the only polymeric unit of the polymer, which may be “capped” at either end with a suitable terminus. In certain embodiments, the polymer further comprises a substituent comprising a tissue-specific or cell-specific targeting moiety.

In some embodiments, the polymer has structure of Formula 1A:

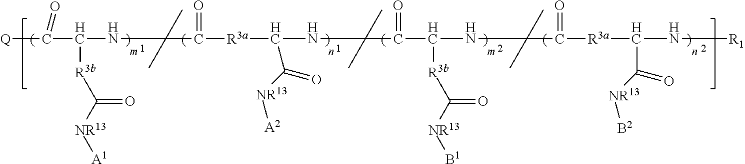

wherein Q is of the formula:

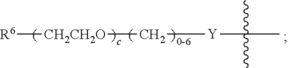

### or Formula 1A1:

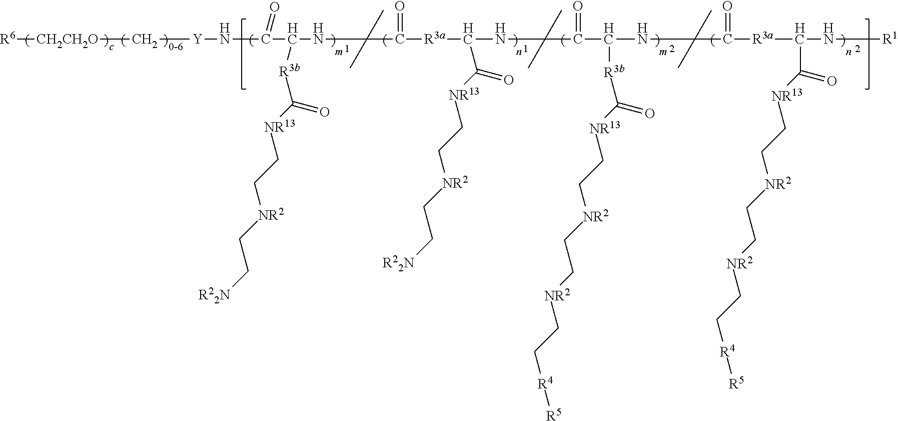

wherein
c is an integer from 0 to 50;
Y is optionally present and is a cleavable linker as previously described;
R¹ is hydrogen, an aryl group, a heterocyclic group (e.g., aromatic or non-aromatic), a C₁-C₁₂ alkyl group, alkenyl group, cycloalkyl group, or cycloalkenyl group, or a C₁-C₁₂ linear or branched alkyl group optionally substituted with one or more substituents, such as one or more amines;
R⁶ is hydrogen, an amino group, an aryl group, a heterocyclic group (e.g., aromatic or non-aromatic), a C₁-C₁₂ alkyl group, alkenyl group, cycloalkyl group, or cycloalkenyl group, a C₁-C₁₂ linear or branched alkyl group optionally substituted with one or more amines; or a tissue-specific or cell-specific targeting moiety;
each instance of R¹³ is independently hydrogen or a C₁-C₁₂ alkyl group, alkenyl group, cycloalkyl group, or cycloalkenyl group;
and all other substituents (e.g., m¹, n¹, m², n², R², R^{3a}, R^{3b}, R⁴, R⁵, and R¹³) are as previously described.

Similarly, the polymer can have the structure of Formula 1.1A:

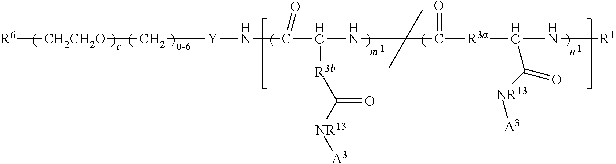

or, where the backbone is in an alpha, beta configuration, as Formula 1.2A:

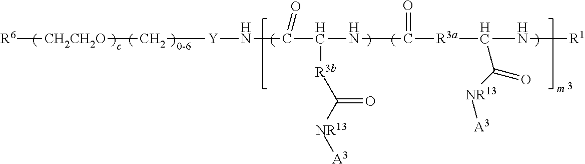

wherein,
c is an integer from 0 to 50;
Y is optionally present and is a cleavable linker as previously described;
R¹ is hydrogen, an aryl group, a heterocyclic group (e.g., aromatic or non-aromatic), a C₁-C₁₂ alkyl group, alkenyl group, cycloalkyl group, or cycloalkenyl group, or a C₁-C₁₂ linear or branched alkyl group optionally substituted with one or more substituents, such as one or more amines;
R⁶ is hydrogen, an amino group, an aryl group, a heterocyclic group (e.g., aromatic or non-aromatic), a C₁-C₁₂ alkyl group, alkenyl group, cycloalkyl group, or cycloalkenyl group, a C₁-C₁₂ linear or branched alkyl group optionally substituted with one or more amines; or a tissue-specific or cell-specific targeting moiety;
each instance of R¹³ is independently hydrogen or a C₁-C₁₂ alkyl group, alkenyl group, cycloalkyl group, or cycloalkenyl group;
and all other substituents (e.g., m³, R^{3a}, R^{3b}, and A³) are as previously described.

In some embodiments of the foregoing, R¹ and/or R⁶ is a C₁-C₁₂ (e.g., a C₁-C₁₀ alkyl group; a C₁-C₈alkyl group; a C₁-C₆ alkyl group; a C₁-C₄ alkyl group, a C₁-C₃ alkyl group, or a C₁ or C₂ alkyl group) linear or branched alkyl group substituted with one or more substituents. In certain embodiments, the heteroalkyl or alkyl group comprises or is substituted with one or more amines, for instance, from 1 to 8 or 2 to 8 (i.e., 1, 2, 3, 4, 5, 6, 7, or 8) secondary or tertiary amines. The amines can be a part of the heteroalkyl backbone chain or pendant substituents.

Non-limiting examples of the polymers provided herein include, for instance:

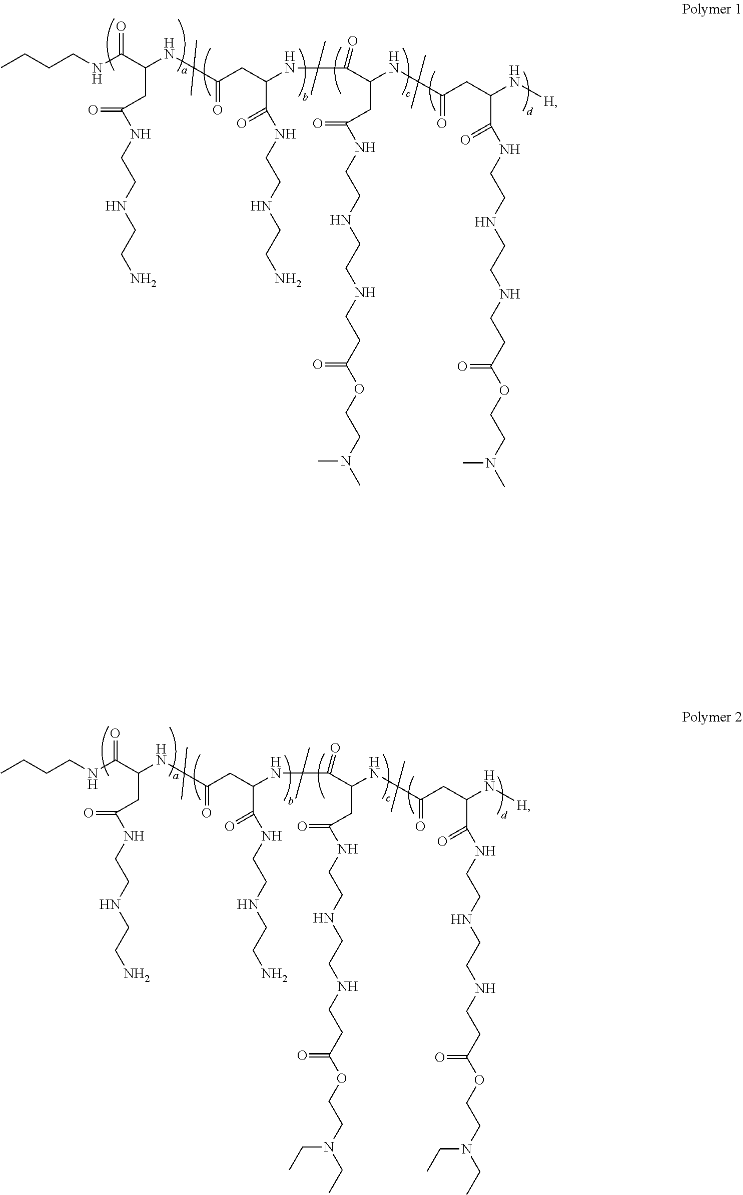

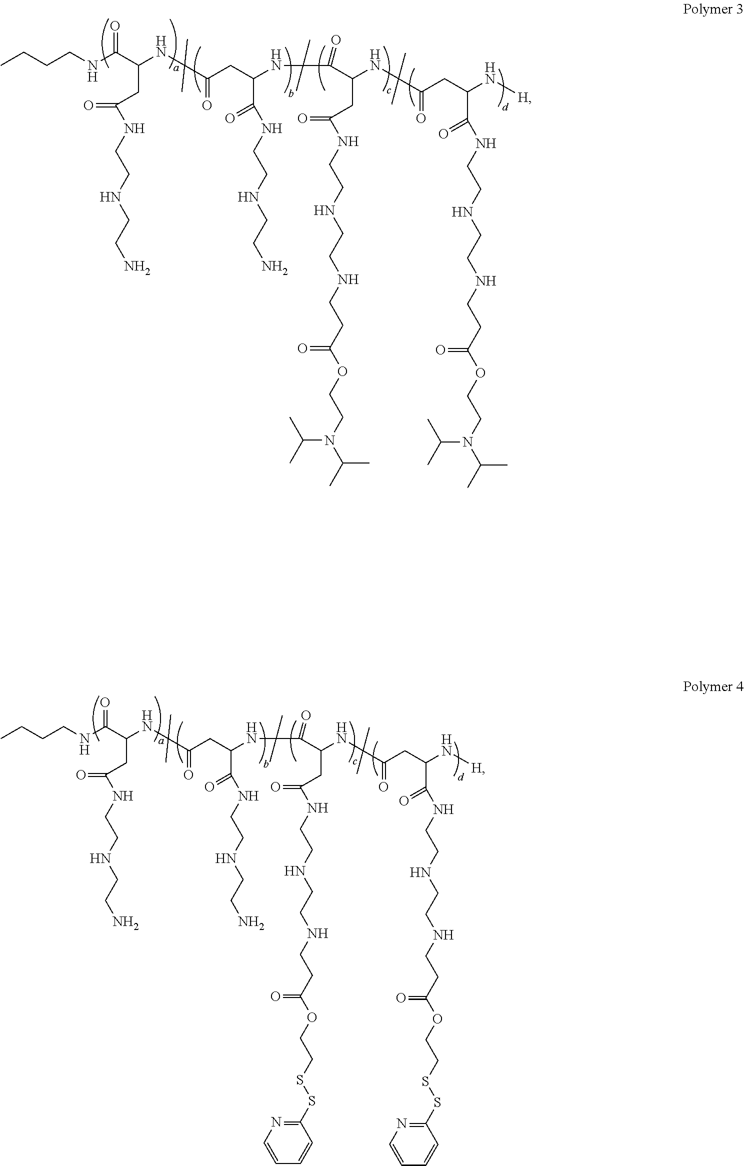

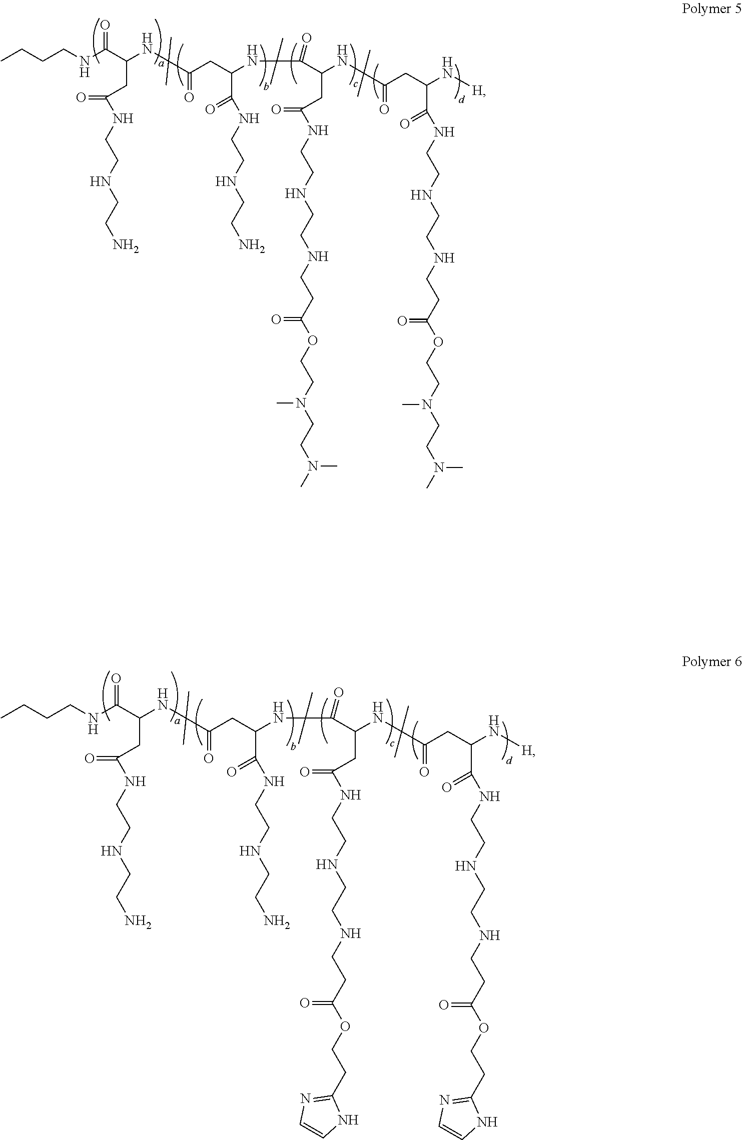

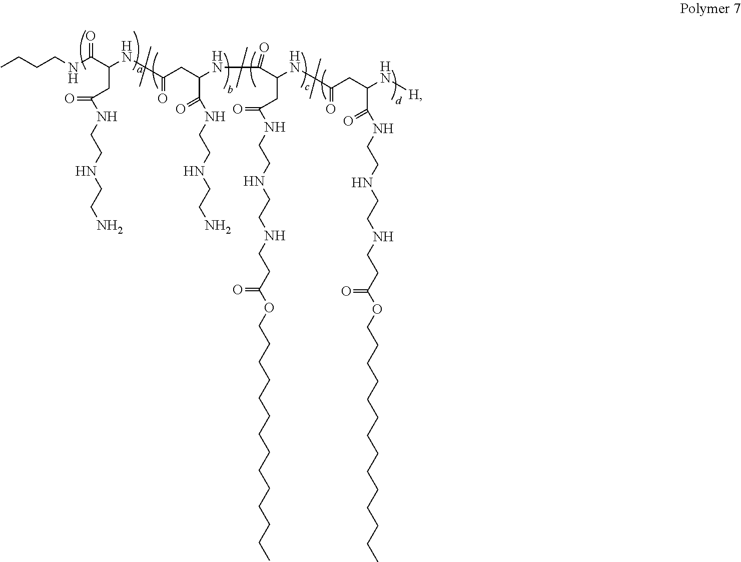

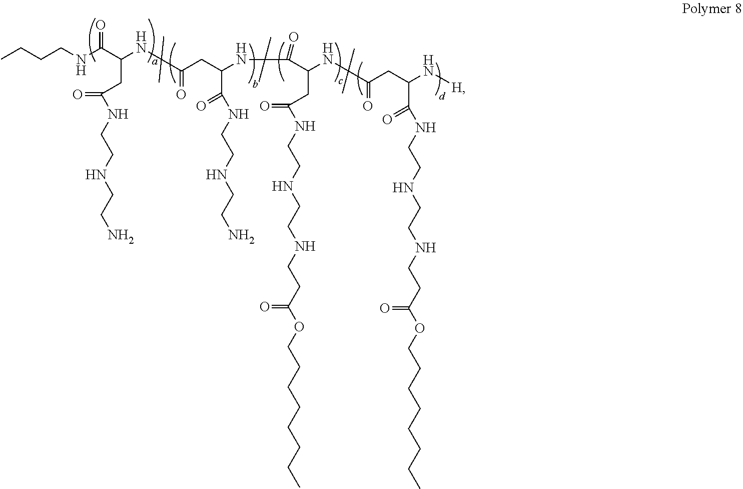

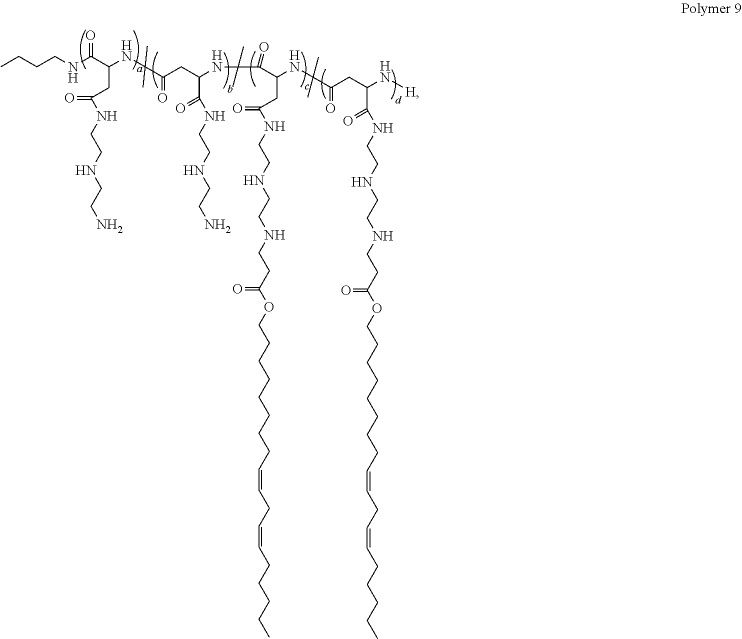

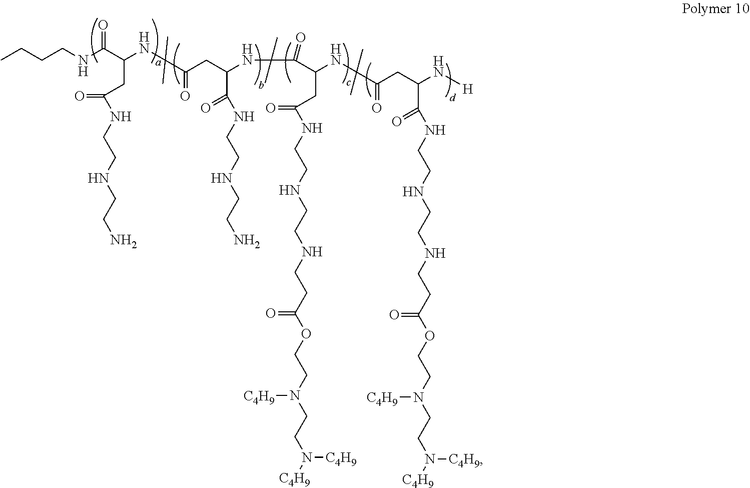

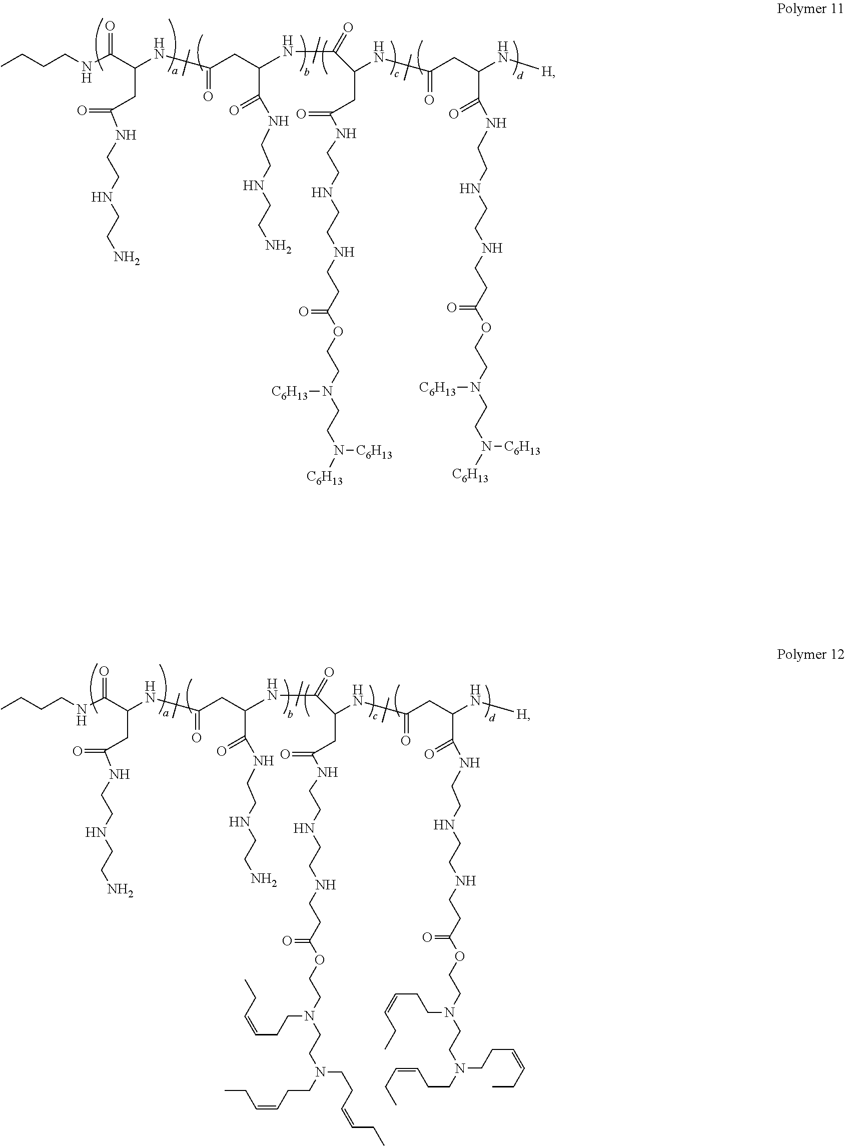

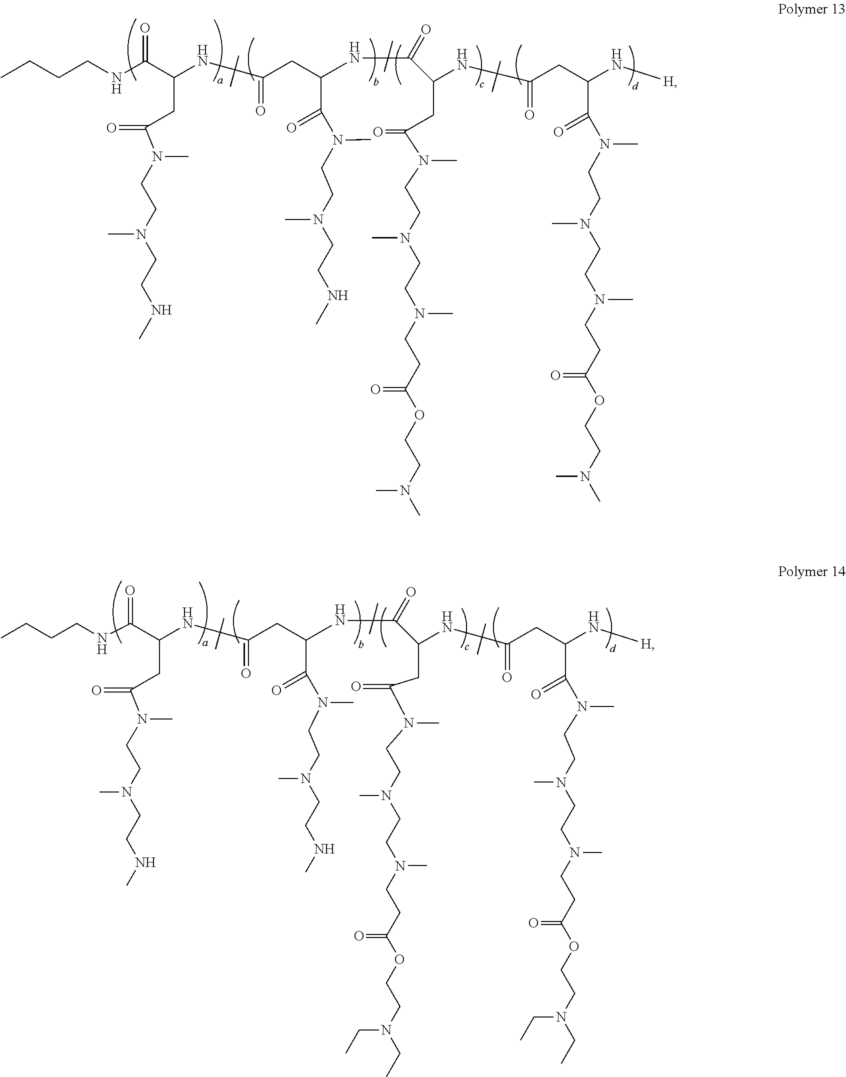

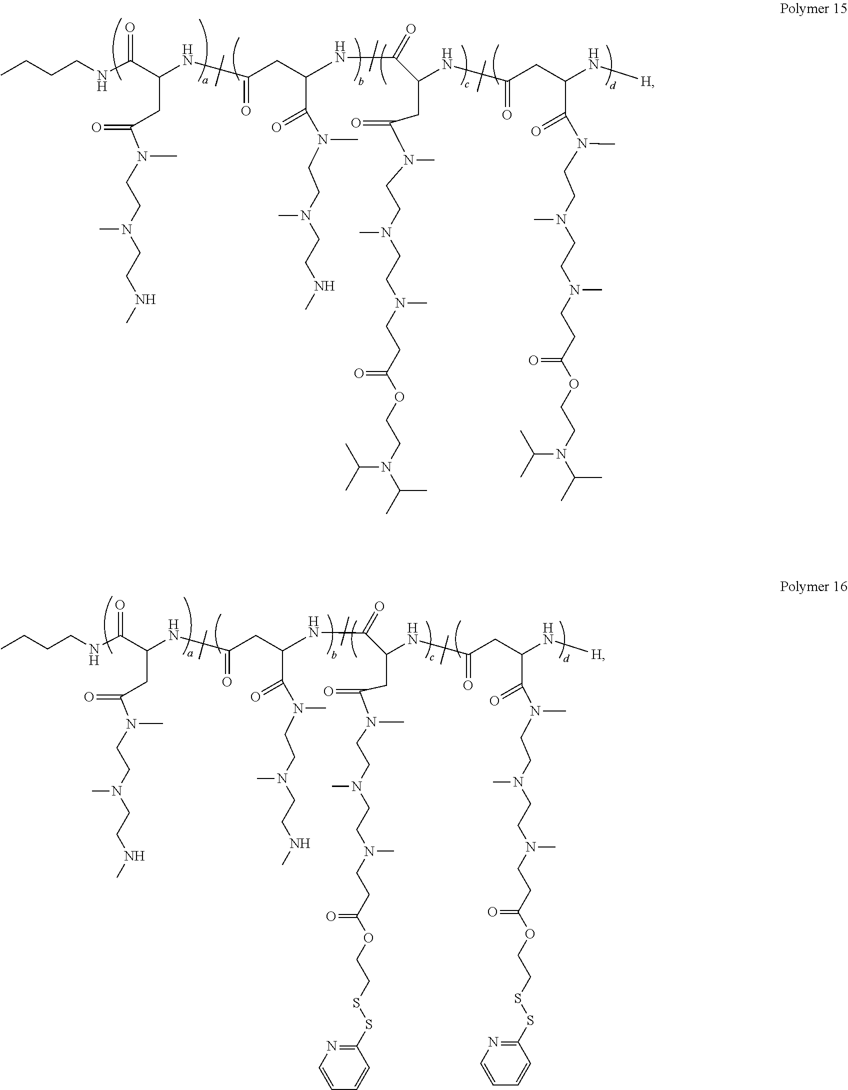

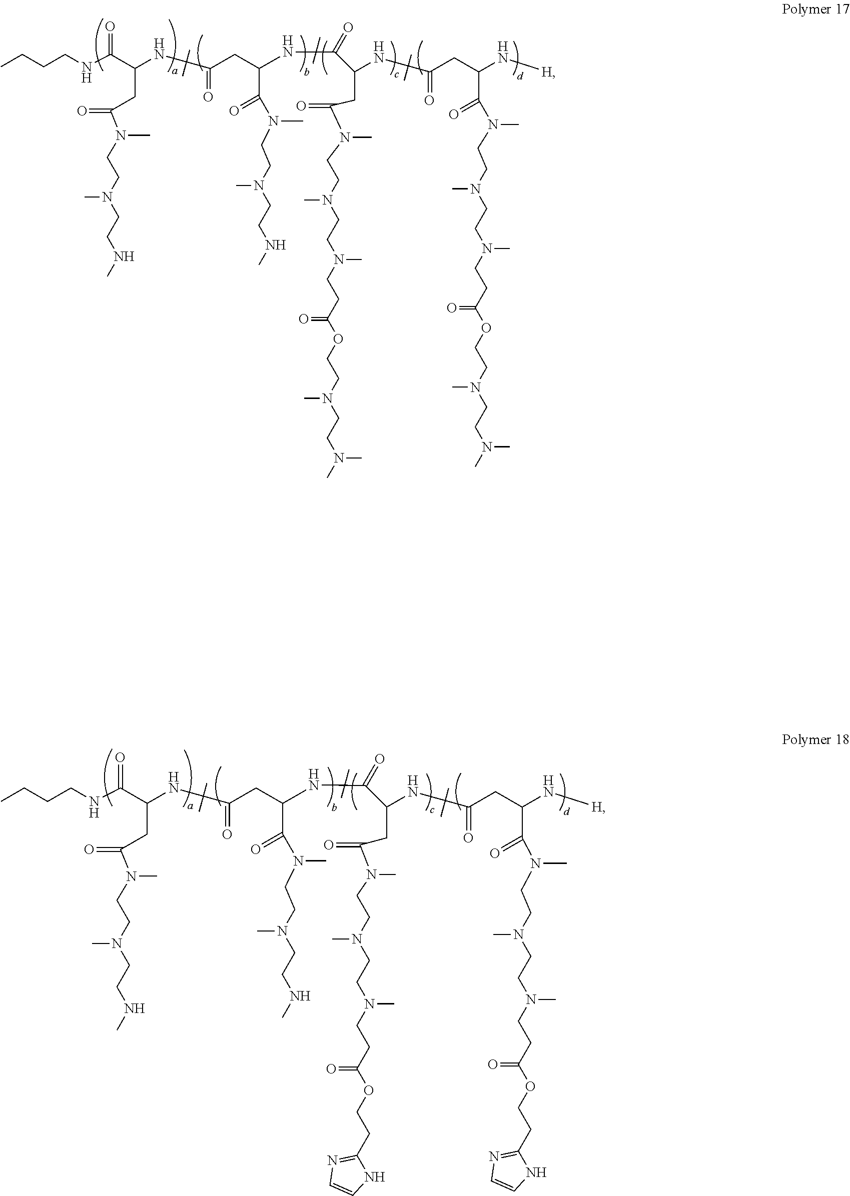

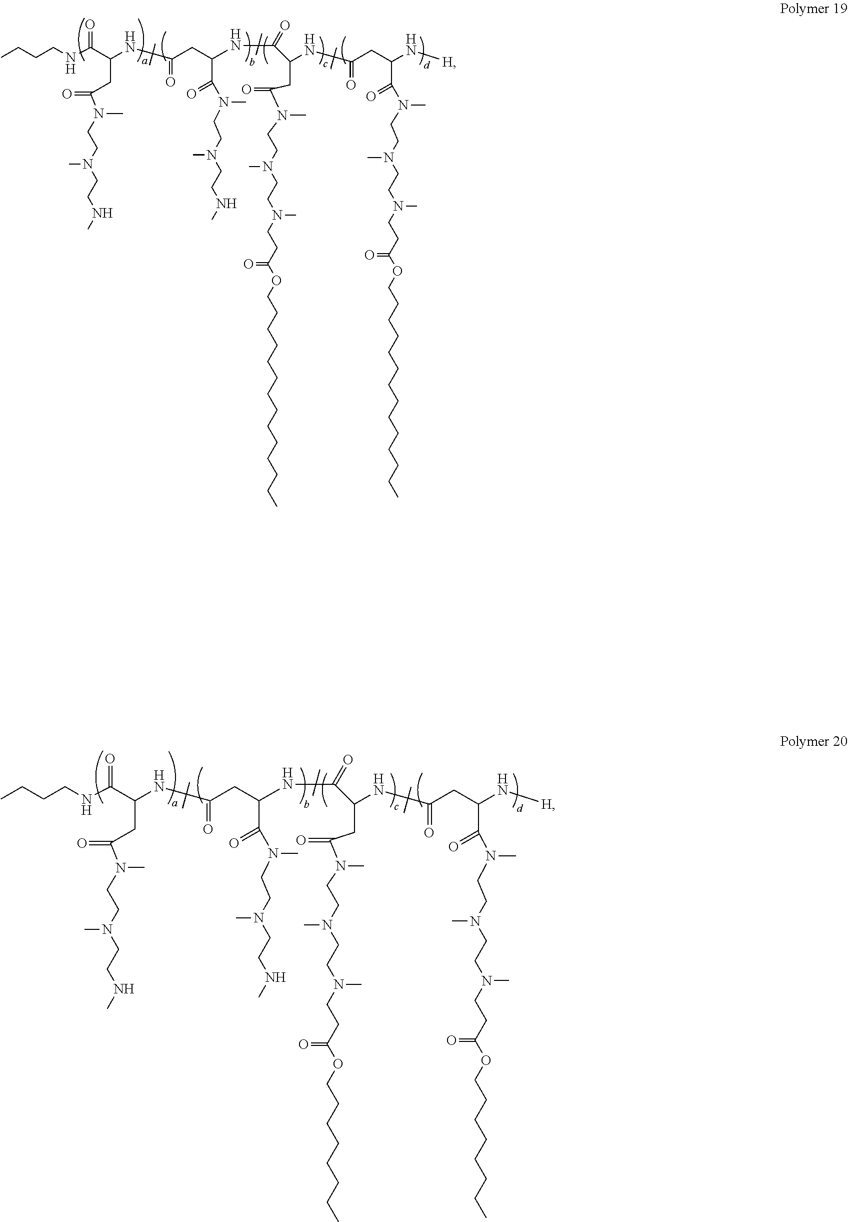

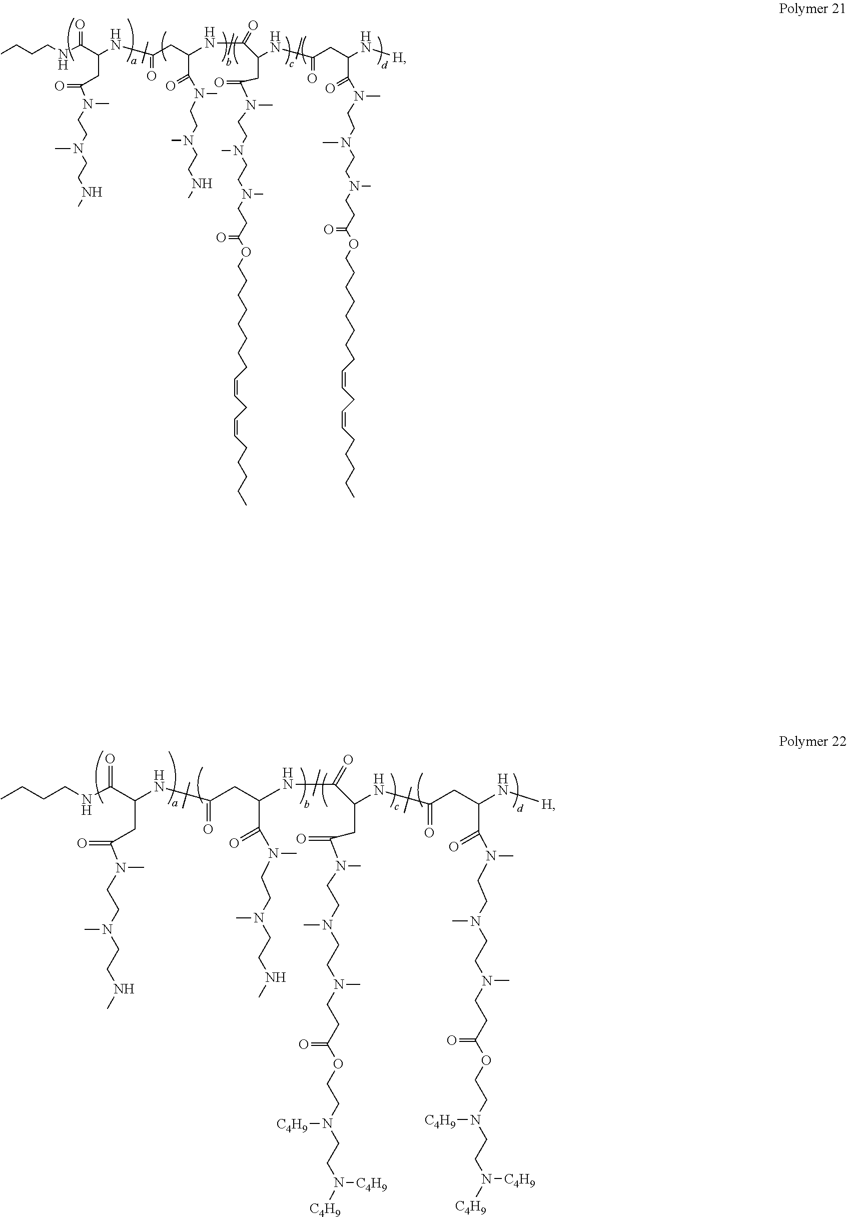

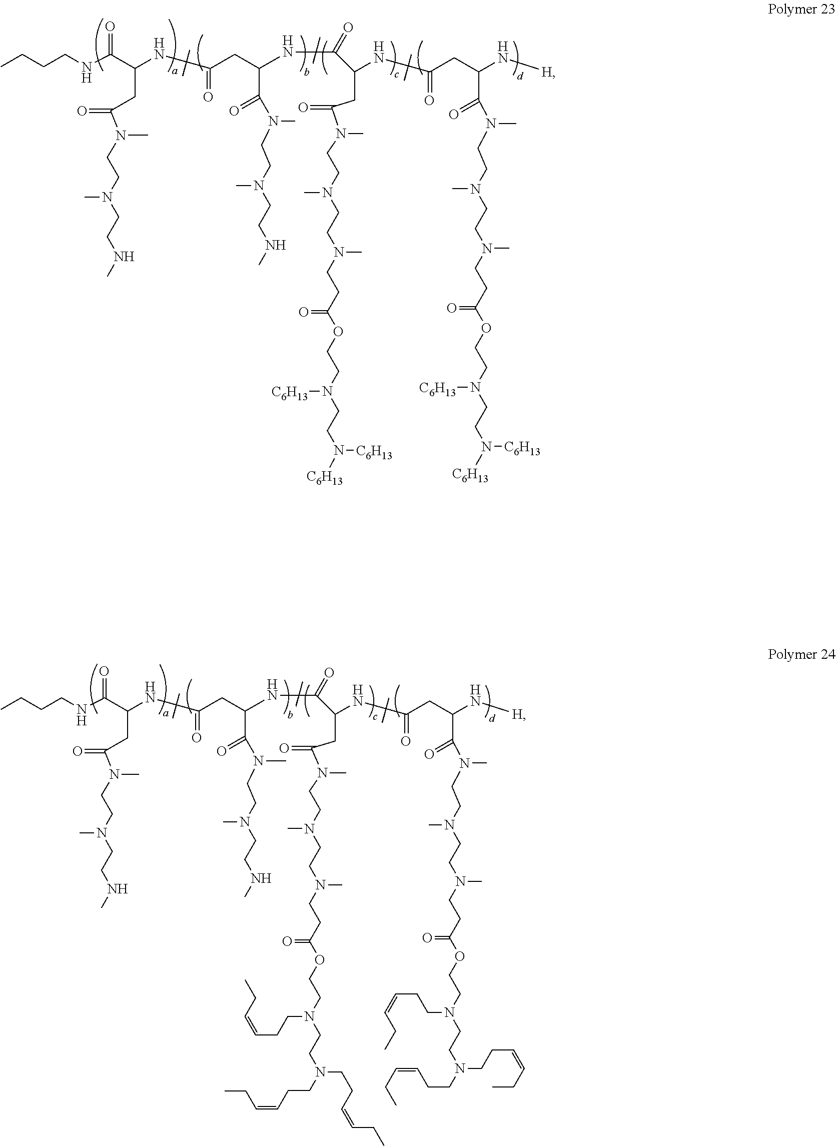

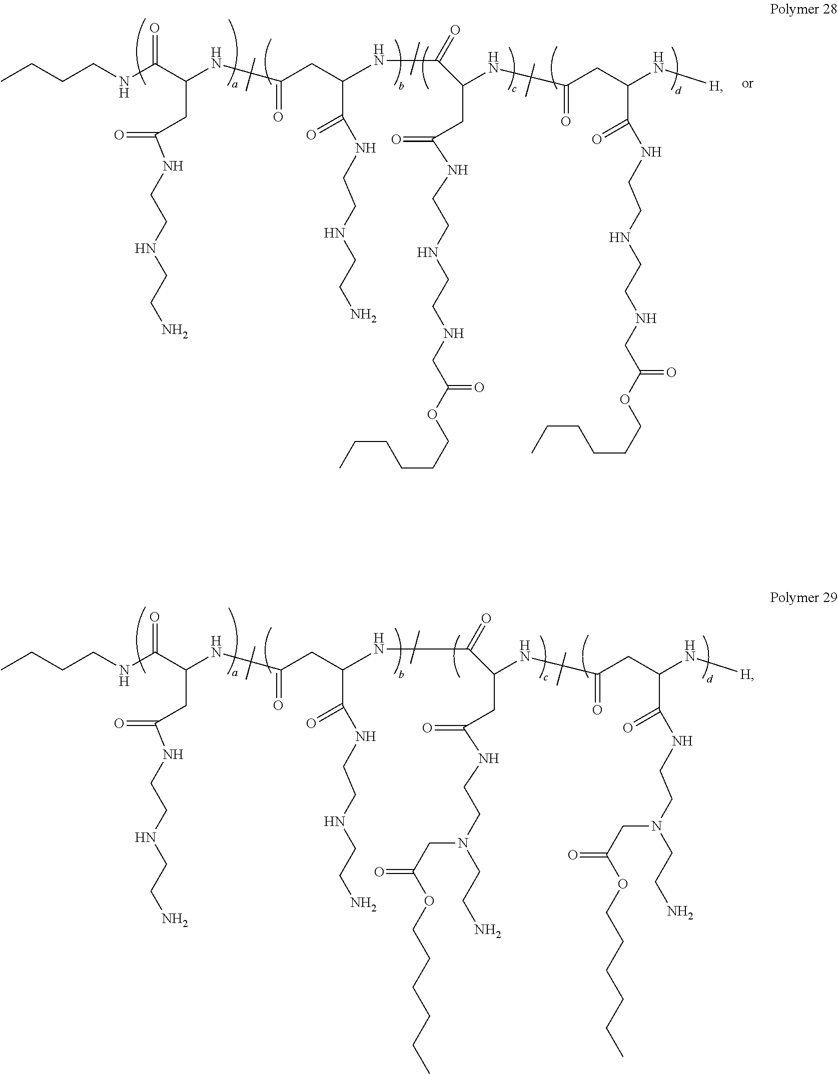

wherein each of a, b, c, and d is independently an integer from 0 to 1000, such as 0-500, 0-200, 0-100, or 0-50, provided that (a+b+c+d)>about 5. In some embodiments, (a+b) is from about 0 to about 75 (e.g., about 5 to about 75, about 20 to about 75, about 40 to about 75, about 40 to about 60, about 50 to about 75, about 60 to about 75, or about 70 to about 75), and (c+d) is from about 5 to about 80 (e.g., about 5 to about 75, about 5 to about 60, about 5 to about 40, about 20 to about 40, about 5 to about 30, about 5 to about 20, or about 5 to about 10). In certain embodiments, (a+b) is greater than (c+d). For instance, in some embodiments, the ratio of (a+b)/(c+d) is from about 1 to about 3 (e.g., (a+b) is about 55 and (c+d) is about 25), or the ratio of (a+b)/(c+d) is from about 3 to about 10 (e.g., (a+b) is about 70 and (c+d) is about 10). In other embodiments, (a+b) is less than (c+d) such that the ratio of (a+b)/(c+d) is less than 1 (e.g., about 0.1 or more, but less than 1). Again, the indication of the number of units (“a”, “b”, “c”, and “d”) in these exemplary polymers does not imply a block co-polymer structure; rather, these numbers indicate the number of units overall, which units can be randomly arranged as indicated by the “/” symbols in the formulas.

The invention also provides a polymer comprising a structure of Formula 2:

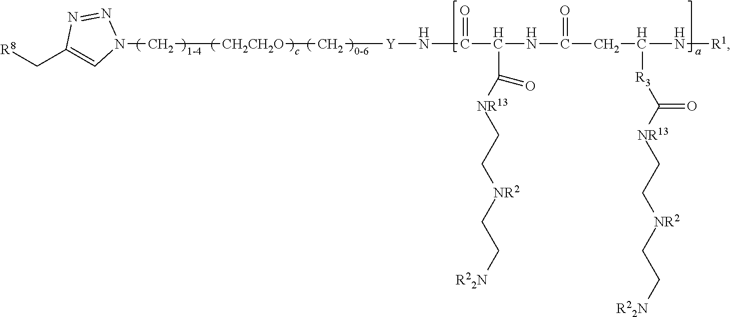

wherein:
a is an integer from 10 to 2,000;
c is an integer from 0 to 50;
Y is optionally present and is a cleavable linker as previously described herein;
R¹, R², and R¹³ and all other substituent groups are as previously described herein with respect to the other polymer formulas (e.g., Formulas 1, 1A, 1A1, 1.1A, and 1.2A;
and R⁸ is a is a tissue-specific or cell-specific targeting moiety.

The invention also provides a polymer comprising a structure of Formula 4:

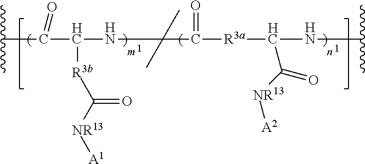

wherein,
each of m¹ and n¹ is an integer from 0 to 1000; provided that the sum of m¹+n¹ is greater than 2;
the symbol “/” indicates that the units separated thereby are linked randomly or in any order;
A¹ and A² are each independently a group of formula

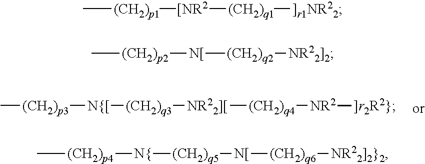

wherein p1 to p4, q1 to q6, and r1 and r2 are each independently an integer of 1 to 5;
each instance of R² is independently hydrogen, a C₁-C₁₂ alkyl group, C₁-C₁₂ alkenyl group, C₃-C₁₂ cycloalkyl group, or C₃-C₁₂ cycloalkenyl group, provided that A¹ and A² comprise at least one (e.g., at least two, at least three, at least four, or even at least five tertiary amines (e.g., at least some instances of R² are a C₁-C₁₂ alkyl group, alkenyl group, cycloalkyl group, or cycloalkenyl group);
R^{3a} and R^{3b} are each independently a methylene or ethylene group; and
each instance of R¹³ is independently hydrogen or a C₁-C₁₂ alkyl group, alkenyl group, cycloalkyl group, or cycloalkenyl group.

The invention further provides a polymer having the structure of Formula 4A:

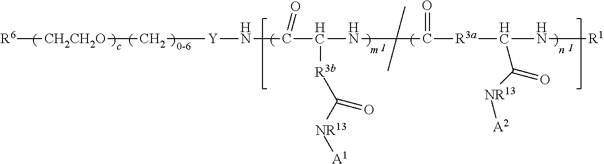

wherein,
c is an integer from 0 to 50;
Y is optionally present and is a cleavable linker;
each of m¹ and n¹ is an integer from 0 to 1000; provided that the sum of m¹+n¹ is greater than 2;
the symbol “/” indicates that the units separated thereby are linked randomly or in any order;
A¹ and A² are each independently a group of formula

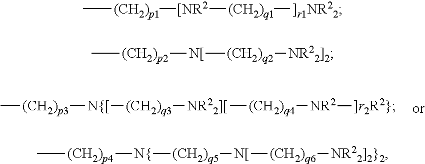

wherein p1 to p4, q1 to q6, and r1 and r2 are each independently an integer of 1 to 5;
R¹ is hydrogen, an aryl group, a heterocyclic group (e.g., aromatic or non-aromatic), a C₁-C₁₂ alkyl group, alkenyl group, cycloalkyl group, or cycloalkenyl group, or a C₁-C₁₂ linear or branched alkyl group optionally substituted with one or more substituents;
each instance of R² is independently hydrogen, a C₁-C₁₂ alkyl group, C₁-C₁₂ alkenyl group, C₃-C₁₂ cycloalkyl group, or C₃-C₁₂ cycloalkenyl group, provided that A¹ and A² comprise at least one (e.g., at least two, at least three, at least four, or even at least five tertiary amines (e.g., at least some instances of R² are a C₁-C₁₂ alkyl group, alkenyl group, cycloalkyl group, or cycloalkenyl group);
R^{3a} and R^{3a} are each independently a methylene or ethylene group;
R⁶ is hydrogen, an amino group, an aryl group, a heterocyclic group (e.g., aromatic or non-aromatic), a C₁-C₁₂ alkyl group, alkenyl group, cycloalkyl group, or cycloalkenyl group, a C₁-C₁₂ linear or branched alkyl group optionally substituted with one or more amines; or a tissue-specific or cell-specific targeting moiety; and
each instance of R¹³ is independently hydrogen or a C₁-C₁₂ alkyl group, alkenyl group, cycloalkyl group, or cycloalkenyl group.

In embodiments of polymers comprising a structure of Formula 4 and polymers having the structure of Formula 4A, each instance of R² is independently hydrogen or a C₁-C₁₂ alkyl group, C₁-C₁₂ alkenyl group, C₃-C₁₂ cycloalkyl group, or C₃-C₁₂ cycloalkenyl group, provided that A¹ and A² each comprise at least one tertiary amine (e.g., at least two, at least three, at least four, or even at least five tertiary amines). In some embodiments, each instance of R² is independently a C₁-C₁₂ alkyl group, alkenyl group, cycloalkyl group, or cycloalkenyl group. Accordingly, in embodiments of polymers comprising a structure of Formula 4 and polymers having the structure of Formula 4A, each nitrogen containing R² substituents is a tertiary amine, except that the terminal amine can be a primary, secondary, or tertiary amine, preferably a secondary or tertiary amine. In some embodiments of polymers comprising a structure of Formula 4 and polymers having the structure of Formula 4A, each of A¹ and A² is a group of formula —(CH₂)₂—NR²—(CH₂)₂—NR²₂, wherein each instance of R² is independently a C₁-C₁₂ alkyl group, alkenyl group, cycloalkyl group, or cycloalkenyl group, except that the terminal amine can be a primary, secondary, or tertiary amine, preferably a secondary or tertiary amine. In certain embodiments of polymers comprising a structure of Formula 4 and polymers having the structure of Formula 4A, each instance of R² is ethyl or methyl, optionally with the exception that the terminal amine can be a primary, secondary, or tertiary amine, preferably a secondary or tertiary amine.

In embodiments of polymers comprising a structure of Formula 4 and polymers having the structure of Formula 4A, each instance of R¹³ is independently hydrogen or a C₁-C₁₂ alkyl group, alkenyl group, cycloalkyl group, or cycloalkenyl group. Generally, each instance of R¹³ is independently hydrogen or a methyl substituent. In certain embodiments, each instance of R¹³ is hydrogen.

According to any of the foregoing, examples of groups A¹ and A² include, for instance, —NH—CH₂—CH₂—N(CH₃)—CH₂—CH₂—N(CH₃)₂; —N(CH₃)—CH₂—CH₂—N(CH₃)—CH₂—CH₂—N(CH₃)₂; —NH—CH₂—CH₂—N(CH₃)—CH₂—CH₂—N(CH₃)—CH₂—CH₂—N(CH₃)₂; —N(CH₃)—CH₂—CH₂—N(CH₃)—CH₂—CH₂—N(CH₃)—CH₂—CH₂—N(CH₃)₂; —NH—CH₂—CH₂—N(CH₃)—CH₂—CH₂—NH(CH₃); —N(CH₃)—CH₂—CH₂—N(CH₃)—CH₂—CH₂—NH(CH₃); —NH—CH₂—CH₂—N(CH₃)—CH₂—CH₂—N(CH₃)—CH₂—CH₂—NH(CH₃); —N(CH₃)—CH₂—CH₂—N(CH₃)—CH₂—CH₂—N(CH₃)—CH₂—CH₂—NH(CH₃).

Non-limiting examples of the polymers provided herein, thus, include, for instance:

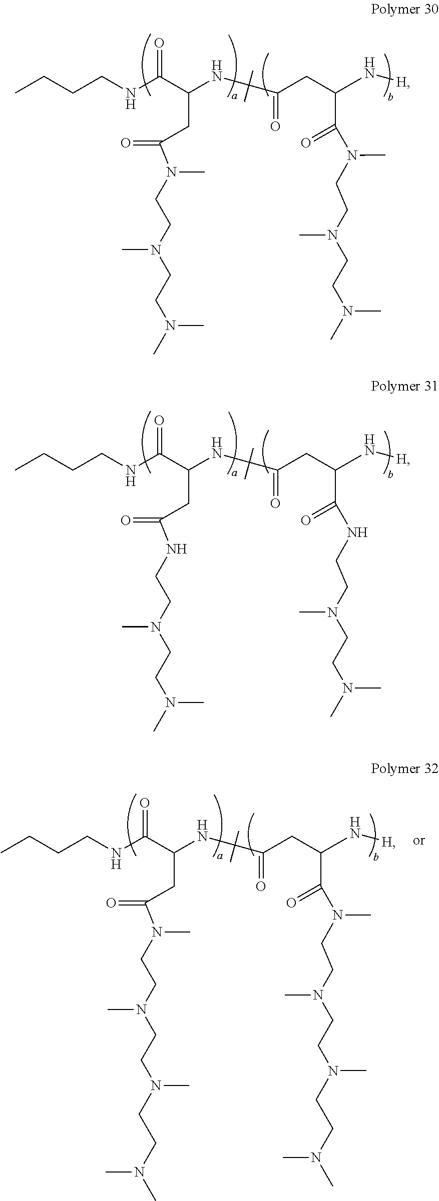

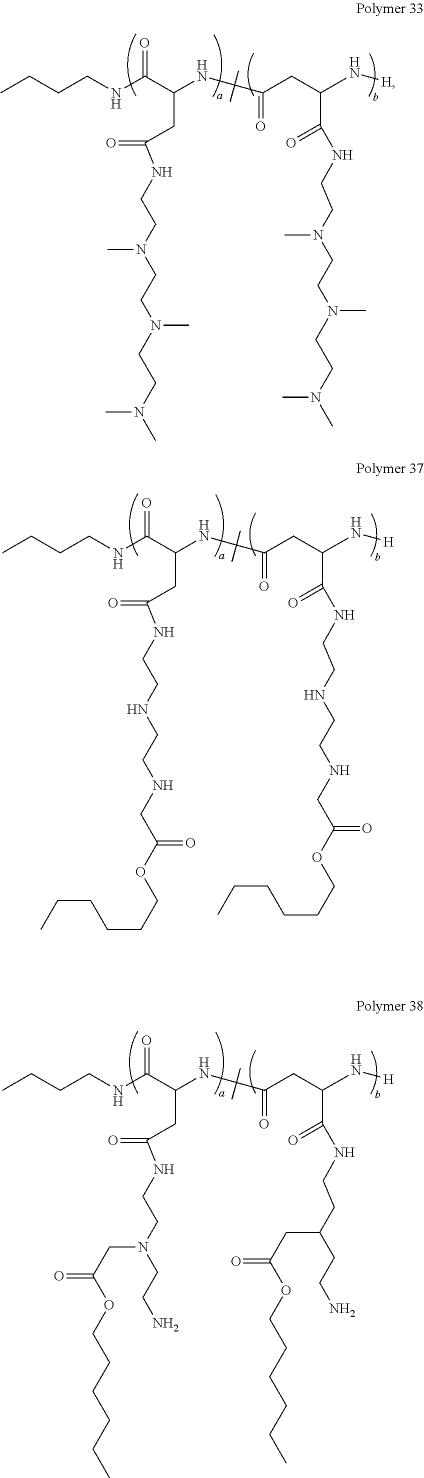

wherein each of a and b is independently an integer from 0 to 1000, such as 0-500, 0-200, 0-100, or 0-50. Thus, either a or b might be 0 in some embodiments, or the polymer might have some ratio of a and b. In some embodiments, (a+b) is about 5 or more (e.g., from about 5 to about 160, about 5 to about 140, about 5 to about 120, about 5 to about 100, about 5 to about 80, or about 5 to about 60) or about 25 or more (e.g., about 25 to about 160, about 25 to about 140, about 25 to about 120, about 25 to about 100, about 25 to about 80, or about 25 to about 60) or even about 50 or more (e.g., about 50 to about 160, about 50 to about 140, about 50 to about 120, about 50 to about 100, or about 50 to about 80). Further examples include polymers 30-33, wherein the terminal tertiary amine of one or both side chains is demethylated to provide a secondary amine (e.g., —NHCH₃):

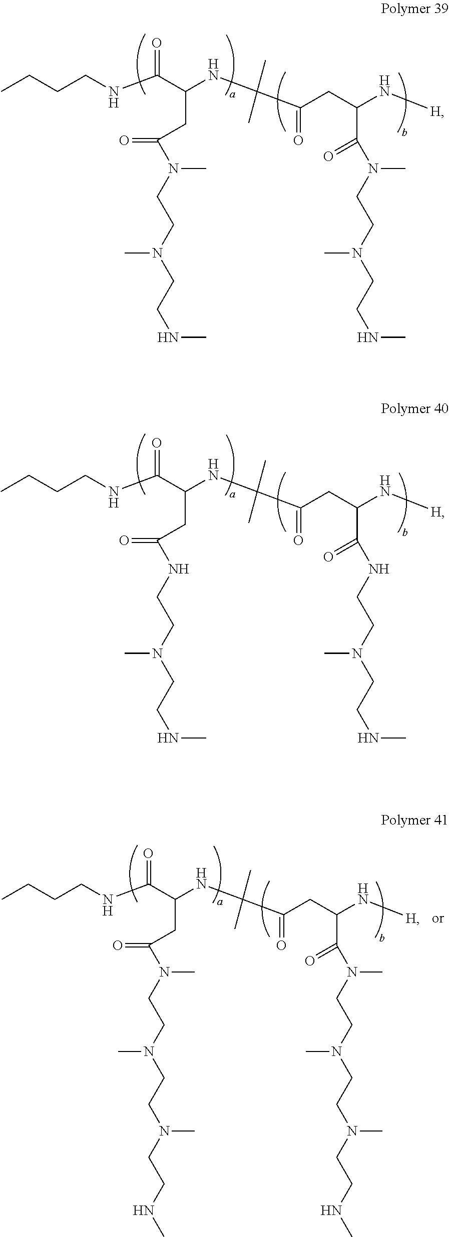

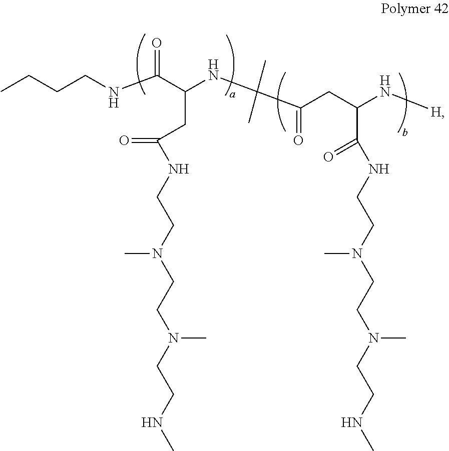

wherein a and b are as described above.

Any of the forgoing polymers can comprise a tissue-specific or cell-specific targeting moiety at a position indicated in the described formulas, or the polymers can be otherwise modified to include a tissue-specific or cell-specific targeting moiety. Methods for attaching the tissue-specific or cell-specific targeting moiety are known in the art, some of which include Michael addition, epoxide ring opening, displacement reactions, or “click” chemistry (e.g., CuAAC, SPAAC, SPANC, or reaction of strained alkenes) using tissue-specific or cell-specific targeting moiety with appropriate functional groups. The tissue-specific or cell-specific targeting moiety can be any small molecule, protein (e.g., antibody or antigen), amino acid sequence, sugar, oligonucleotide, metal-based nanoparticle, or combination thereof, capable of recognizing (e.g., specifically binding) a given target tissue or cell (e.g., specifically binding a particular ligand, receptor, or other protein or molecule that allows the targeting moiety to discriminate between the target tissue or cell and other non-target tissues or cells). In some embodiments, the tissue-specific or cell-specific targeting moiety is a receptor for a ligand. In some embodiments, the tissue-specific or cell-specific targeting moiety is a ligand for a receptor.

The tissue-specific or cell-specific targeting moiety can be used to target any desired tissue or cell type. In some embodiments, the tissue-specific or cell-specific targeting moiety localizes the polymer to tissues of the peripheral nervous system, the central nervous system, liver, muscle (e.g., cardiac muscle), lung, bone (e.g., hematopoietic cells), or the eye of the subject. In certain embodiments, the tissue-specific or cell-specific targeting moiety localizes the polymer to tumor cells. For example, the tissue-specific or cell-specific targeting moiety can be a sugar that binds to a receptor on a specific tissue or cell.

In some embodiments, the tissue-specific or cell-specific targeting moiety is:

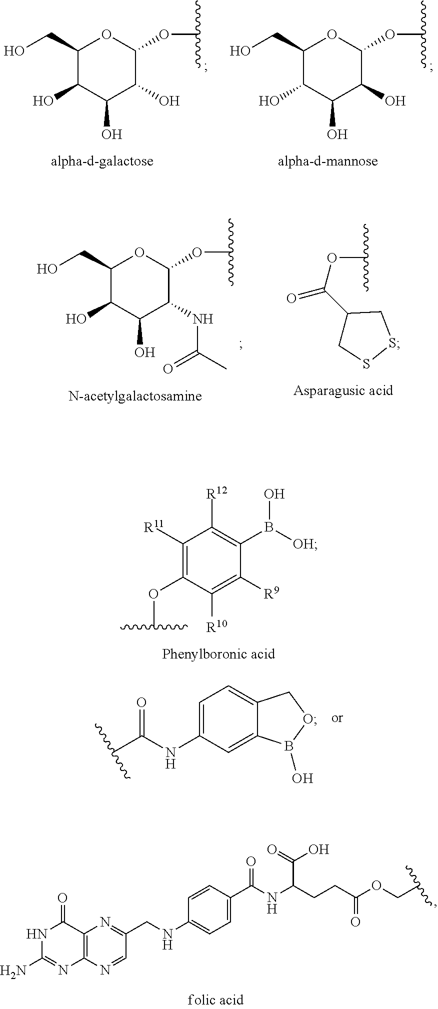

wherein each of R⁹, R¹⁰, R¹¹, and R¹² is independently hydrogen, halogen, C₁-C₄ alkyl, or C₁-C₄ alkoxy, optionally substituted with one or more amino groups. The specified tissue-specific or cell-specific targeting moieties can be chosen to localize the polymer to a tissue described herein. For example, alpha-d-mannose can be used to localize the polymer to the peripheral nervous system, the central nervous system, or immune cells, alpha-d-galactose and N-acetylgalactosamine can be used to localize the polymer to liver hepatocytes, and folic acid can be used to localize the polymer to tumor cells.

Typically, the polymer is cationic (i.e., positively charged at pH 7 and 23° C.). As used herein, “cationic” polymers refer to polymers having an overall net positive charge, whether the polymer comprises only cationic monomer units or a combination of cationic monomer units and non-ionic or anionic monomer units.

In certain embodiments, the polymer has a weight average molecular weight of from about 5 kDa to about 2000 kDa, such as about 10 kDa to about 2,000 kDa. The polymer can have a weight average molecular weight of about 2,000 kDa or less, for example, about 1,800 kDa or less, about 1,600 kDa or less, about 1,400 kDa or less, about 1,200 kDa or less, about 1,000 kDa or less, about 900 kDa, or less, about 800 kDa, or less, about 700 kDa or less, about 600 kDa or less, about 500 kDa or less, about 100 kDa or less, or about 50 kDa or less or even about 40 kDa or less, such as about 30 kDa or less or 25 kDa or less. Alternatively, or in addition, the polymer can have a weight average molecular weight of about 5 kDa or more or about 10 kDa or more, for example, about 50 kDa or more, about 100 kDa or more, about 200 kDa or more, about 300 kDa or more, or about 400 kDa or more. Thus, the polymer can have a weight average molecular weight bounded by any two of the aforementioned endpoints. For example, the polymer can have a weight average molecular weight of from about 5 kDa to about 50 kDa, about 10 kDa to about 50 kDa, from about, from about 10 kDa to about 100 kDa, from about 10 kDa to about 500 kDa, from about 50) kDa to about 500 kDa, from about 100 kDa to about 500 kDa, from about 200 kDa to about 500 kDa, from about 300 kDa to about 500 kDa, from about 400 kDa to about 500 kDa, from about 400 kDa to about 600 kDa, from about 400 kDa to about 700 kDa, from about 400 kDa to about 800 kDa, from about 400 kDa to about 900 kDa, from about 400 kDa to about 1,000 kDa, from about 400) kDa to about 1,200 kDa, from about 400 kDa to about 1,400 kDa, from about 400 kDa to about 1,600 kDa, from about 400 kDa to about 1,800 kDa, from about 400) kDa to about 2,000 kDa, from about 200 kDa to about 2,000 kDa, from about 500 kDa to about 2,000 kDa, or from about 800 kDa to about 2,000 kDa.

The weight average molecular weight can be determined by any suitable technique. Generally, the weight average molecular weight is determined using size exclusion chromatography equipped with a column, selected from TSKgel Guard, GMPW, GMPW, G1000PW, and a Waters 2414 (Waters Corporation, Milford, Massachusetts) refractive index detector. Moreover, the weight average molecular weight is determined from calibration with polyethylene oxide/polyethylene glycol standards ranging from 150-875,000 Daltons.

### Methods of Preparation

The polymers provided herein can be provided by any suitable method. In some embodiments, the polymers can be prepared by a method that comprises modifying at least a portion of groups A¹ and/or A² of a polymer comprising a structure of Formula 3:

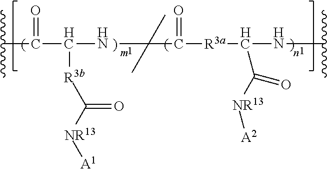

wherein:
each of m¹ and n¹ is an integer from 0 to 1000, provided that m¹+n¹ is greater than 5;
the symbol “/” indicates that the units separated thereby are linked randomly or in any order;
R^{3a} and R^{3b} are each independently a methylene or ethylene group;
each instance of R¹³ is independently hydrogen or a C₁-C₁₂ alkyl group, alkenyl group, cycloalkyl group, or cycloalkenyl group;
A¹ and A² are each independently a group of formula

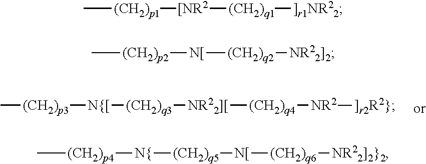

wherein p1 to p4, q1 to q6, and r1 and r2 are each independently an integer of 1 to 5; and each instance of R² is independently hydrogen or a C₁-C₁₂ alkyl group, alkenyl group, cycloalkyl group, or cycloalkenyl group,
   to produce a polymer comprising a structure of Formula 1:

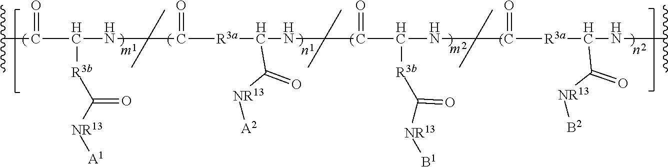

wherein m¹ and n¹ is an integer from 0 to 1000;
each of m² and n² is an integer from 0 to 1000, provided that the sum of m²+n² is greater than 5;
the symbol “/” indicates that the units separated thereby are linked randomly or in any order;
R^{3a} and R^{3b} are each independently a methylene or ethylene group;
each instance of R¹³ is independently hydrogen or a C₁-C₁₂ alkyl group, alkenyl group, cycloalkyl group, or cycloalkenyl group;
A¹ and A² are each independently a group of formula

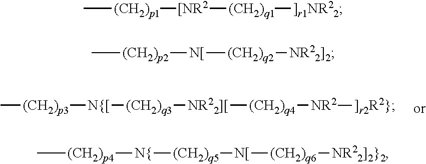

B¹ and B² are each independently

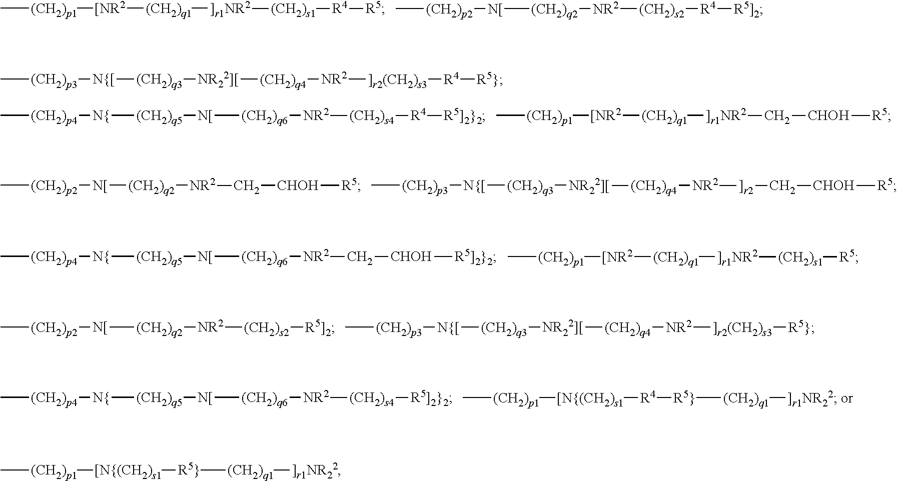

wherein p1 to p4, q1 to q6, r1 and r2, and s1 to s4 are each independently an integer of 1 to 5; each instance of R² is independently hydrogen or a C₁-C₁₂ alkyl group, alkenyl group, cycloalkyl group, or cycloalkenyl group; each instance of R⁴ is independently —C(O)O—, —C(O)NH—, or —S(O)(O)—; and each instance of R⁵ is independently an alkyl group, cycloalkyl group, alkenyl group, cycloalkenyl group, aryl group, heteroalkyl group, heterocyclic group, or combination thereof optionally comprising from 1 to 8 or 2 to 8 secondary or tertiary amines or a substituent comprising a tissue-specific or cell-specific targeting moiety.

The polymer comprising a structure of Formula 1 produced by the method can be any polymer of Formula 1, including Formulas 1.1, 1.2, 1A1, 1A, 1.1A, 1.2A, 4, or 4A as well as any and all embodiments thereof as described with respect to the polymer of the invention.

The polymer comprising a structure of Formula 3 can be any suitable polymer meeting this criteria. In some embodiments, the polymer could instead be described as Formula 3.1:

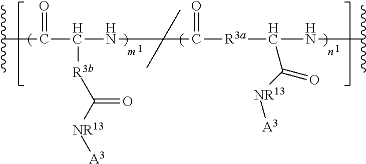

or, where the backbone is in an alpha, beta configuration, as Formula 3.2:

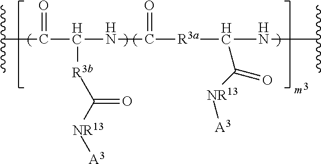

wherein,
m³ is an integer from 5-2000 (e.g., 5-1000, 5-500, 5-100, 25-2000, 25-500, 25-100, 50-2000, 50-1000, 50-500, or 50-100);
the symbol “/” indicates that the units separated thereby are linked randomly or in any order;
R^{3a} and R^{3b} are each independently a methylene or ethylene group;
each instance of R¹³ is independently hydrogen or a C₁-C₁₂ alkyl group, alkenyl group, cycloalkyl group, or cycloalkenyl group;
each A³ is independently a group of formula

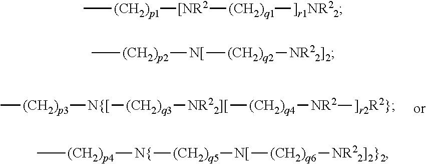

wherein p1 to p4, q1 to q6, and r1 and r2 are each independently an integer of 1 to 5; and each instance of R² is independently hydrogen or a C₁-C₁₂ alkyl group, alkenyl group, cycloalkyl group, or cycloalkenyl group. In this case, the method can comprise modifying at least a portion of the A³ groups sufficient to provide a polymer with a structure of Formula 1.1 or 1.2, as described herein. An example of a polymer comprising one of the following structures is pAsp(DET) (poly (2-[(2-aminoethyl)amino]ethyl aspartamide)) or PEG-pAsp(DET) (polyethylene glycol-b-poly {N′-[N-(2-aminoethyl)-2-aminoethyl]aspartamide}).

The groups designated A¹ and/or A² (or group A³ of the polymer of Formula 3.1 or 3.2) can be modified by any suitable means to produce groups designated B¹ and/or B² (or group A³ of the polymer of Formula 1.1 or 1.2). For example, the groups designated A¹ and/or A² (or group A³ of the polymer of Formula 3.1 or 3.2) can be modified by a Michael addition reaction, an epoxide opening, or a displacement reaction. In preferred embodiments, the groups designated A¹ and/or A² (or group A³ of the polymer of Formula 3.1 or 3.2) are modified by a Michael addition reaction.

In one embodiment, groups A¹ and/or A² of the polymer comprising a structure of Formula 3, or group A³ of the polymer of Formula 3.1 or 3.2, are modified by a Michael addition reaction between the polymer comprising the structure of Formula 3 and α,β-unsaturated carbonyl compound. As used herein, the term “Michael addition” refers to a nucleophilic addition of a nucleophile of the polymer (e.g., a carbanion, an oxygen anion, a nitrogen anion, an oxygen atom, a nitrogen atom, or a combination thereof) to an α,β-unsaturated carbonyl compound. Accordingly, the Michael addition reaction is between the polymer comprising the structure of Formula 3 and an α,β-unsaturated carbonyl compound. In some embodiments, the nucleophile of the polymer is a nitrogen anion, a nitrogen atom, or a combination thereof.

The α,β-unsaturated carbonyl compound can be any α,β-unsaturated carbonyl compound capable of accepting a Michael addition from a nucleophile. In some embodiments, the α,β-unsaturated carbonyl compound is an acrylate, an acrylamide, a vinyl sulfone, or a combination thereof. Accordingly, the Michael addition reaction can be between the polymer comprising the structure of Formula 3 and an acrylate, an acrylamide, a vinyl sulfone, or a combination thereof. Thus, in some embodiments, the method comprises contacting the polymer comprising the structure of Formula 3 and an acrylate; contacting the polymer comprising the structure of Formula 3 and an acrylamide; or contacting the polymer comprising the structure of Formula 3 and a vinyl sulfone.

In embodiments where the groups designated A¹ and/or A² (or group A³ of the polymer of Formula 3.1 or 3.2) are modified by a Michael addition reaction, they produce groups designated B¹ and/or B² as described herein with respect to Formula 1, 1.1, 1.2, 1A, 1A1, etc. (or group A³ of the polymer of Formula 1.1 or 1.2). For instance, B¹ and/or B² can be of the formula:

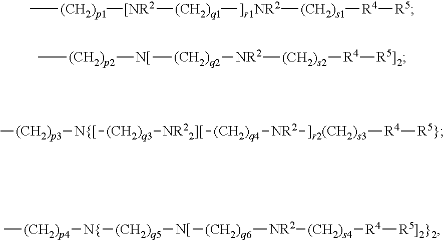

wherein p1 to p4, q1 to q6, r1 and r2, and s1 to s4 are each independently an integer of 1 to 5; each instance of R² is independently hydrogen or a C₁-C₁₂ alkyl group, alkenyl group, cycloalkyl group, or cycloalkenyl group; each instance of R⁴ is
independently —C(O)O—, —C(O)NH—, or —S(O)(O)—; and each instance of R⁵ is independently an alkyl group, cycloalkyl group, alkenyl group, cycloalkenyl group, aryl group, heteroalkyl group, heterocyclic group, or combination thereof optionally comprising from 1 to 8 or 2 to 8 secondary or tertiary amines or a substituent comprising a tissue-specific or cell-specific targeting moiety; or the groups and substituents can be otherwise as described herein with respect to the polymers of the invention.

Examples of acrylates, acrylamides, and vinyl sulfones suitable for use include an acrylate of the formula:

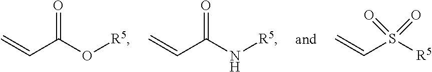

wherein R⁵ is as described with respect to any of Formulas 1, 1.1, 1.2, 1A, 1.1A, or 1.2A.

In some embodiments, the Michael addition reaction is facilitated by an acid and/or base. The acid and/or base can be any suitable acid and/or base with any suitable pKa. The acid and/or base can be an organic acid (e.g., p-toluenesulfonic acid), organic base (e.g., triethylamine), inorganic acid (e.g., titanium tetrachloride), inorganic base (e.g., potassium carbonate), or a combination thereof.

In some embodiments, the Michael addition reaction is facilitated by an acid. The acid can be a Brønsted acid or a Lewis acid. In embodiments where the acid is a Brønsted acid, the acid can be a weak acid (i.e., a pKa of from about 4 to about 7) or a strong acid (i.e., a pKa of from about-2 to about 4). Typically, the acid is a weak acid. In certain embodiments, the acid is a Lewis acid. For example, the acid can be bis(trifluoromethanesulfon)imide or p-toluenesulfonic acid.

In some embodiments, the Michael addition reaction is facilitated by a base. The base can be a weak base (i.e., a pKa of from about 7 to about 12) or a strong base (i.e., a pKa of from about 12 to about 50). Typically, the base is a weak base. For example, the base can be triethylamine, diisopropylethylamine, pyridine, N-methyl morpholine, or N,N-dimethyl-piperazine, or derivatives thereof.

In some embodiments, the Michael addition reaction is performed in a solvent. The solvent can be any suitable solvent, or mixture of solvents, capable of solubilizing the polymer and the α,β-unsaturated carbonyl compound to be reacted. For example, the solvent can include water, protic organic solvents, and/or aprotic organic solvents. An exemplary list of solvents includes water, dichloromethane, diethyl ether, dimethyl sulfoxide, acetonitrile, methanol, and ethanol.

In one embodiment, groups A¹ and/or A² (or group A³ of the polymer of Formula 3.1 or 3.2) of the polymer are modified by an epoxide opening reaction between the polymer and an epoxide compound. As used herein, the term “epoxide opening” refers to a nucleophilic addition of a nucleophile of the polymer (e.g., a carbanion, an oxygen anion, a nitrogen anion, an oxygen atom, a nitrogen atom, or a combination thereof) to an epoxide compound, thereby opening the epoxide. Accordingly, the epoxide opening reaction is between the polymer and an epoxide compound. In some embodiments, the nucleophile of the polymer is a nitrogen anion, a nitrogen atom, or a combination thereof.

In embodiments where the groups designated A¹ and/or A² (or group A³ of the polymer of Formula 3.1 or 3.2) are modified by an epoxide opening reaction, they produce groups designated B¹ and/or B² (or group A³ of the polymer of Formula 1.1 or 1.2) of the formula:

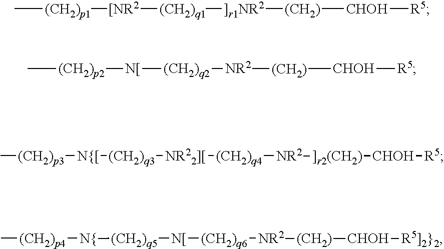

wherein p1 to p4, q1 to q6, and r1 and r2 are each independently an integer of 1 to 5; each instance of R² is independently hydrogen or a C₁-C₁₂ alkyl group, alkenyl group, cycloalkyl group, or cycloalkenyl group; and each instance of R⁵ is independently an alkyl group, cycloalkyl group, alkenyl group, cycloalkenyl group, aryl group, heteroalkyl group, heterocyclic group, or combination thereof optionally comprising from 1 to 8 or 2 to 8 secondary or tertiary amines or a substituent comprising a tissue-specific or cell-specific targeting moiety.

Examples of epoxides suitable for use include epoxides of the formula:

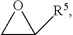

wherein R⁵ is as described with respect to any of the polymers of the invention (e.g., Formulas 1, 1.1, 1.2, 1A, 1.1A, or 1.2A).

In some embodiments, the epoxide opening reaction is facilitated by an acid and/or base. The acid and/or base can be any suitable acid and/or base with any suitable pKa. The acid and/or base can be an organic acid (e.g., p-toluenesulfonic acid), organic base (e.g., triethylamine), inorganic acid (e.g., titanium tetrachloride), inorganic base (e.g., potassium carbonate), or a combination thereof.

In some embodiments, the epoxide opening reaction is facilitated by an acid. The acid can be a Brønsted acid or a Lewis acid. In embodiments where the acid is a Brønsted acid, the acid can be a weak acid (i.e., a pKa of from about 4 to about 7) or a strong acid (i.e., a pKa of from about-2 to about 4). Typically, the acid is a weak acid. In certain embodiments, the acid is a Lewis acid. For example, the acid can be bis(trifluoromethanesulfon)imide or p-toluenesulfonic acid.

In some embodiments, the epoxide opening reaction is facilitated by a base. The base can be a weak base (i.e., a pKa of from about 7 to about 12) or a strong base (i.e., a pKa of from about 12 to about 50). Typically, the base is a weak base. For example, the base can be triethylamine, diisopropylethylamine, pyridine, N-methyl morpholine, or N,N-dimethyl-piperazine, or derivatives thereof.

In some embodiments, the epoxide opening reaction is performed in a solvent. The solvent can be any suitable solvent, or mixture of solvents, capable of solubilizing the polymer and the epoxide compound to be reacted. For example, the solvent can include water, protic organic solvents, and/or aprotic organic solvents. An exemplary list of solvents includes water, dichloromethane, diethyl ether, dimethyl sulfoxide, acetonitrile, methanol, and ethanol.

In one embodiment, groups A¹ and/or A² (or group A³ of the polymer of Formula 3.1 or 3.2) of the polymer are modified by a displacement reaction between the polymer and a compound comprising a leaving group (e.g., chloride atom, bromide atom, iodide atom, tosylate, triflate, mesylate, etc.). As used herein, the term “displacement” refers to a nucleophilic addition of a nucleophile of the polymer (e.g., a carbanion, an oxygen anion, a nitrogen anion, an oxygen atom, a nitrogen atom, or a combination thereof) to a compound comprising a leaving group. Accordingly, the displacement reaction is between the polymer and a compound comprising a leaving group. In some embodiments, the nucleophile of the polymer is a nitrogen anion, a nitrogen atom, or a combination thereof.

In embodiments where the groups designated A¹ and/or A² (or group A³ of the polymer of Formula 3.1 or 3.2) are modified by a displacement reaction, they produce groups designated B¹ and/or B² (or group A³ of the polymer of Formula 1.1 or 1.2) of the formula:

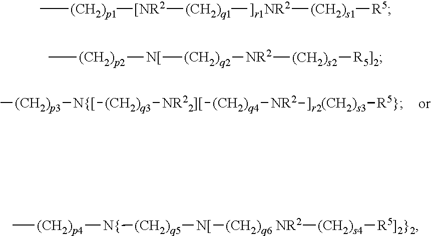

wherein p1 to p4, q1 to q6, r1 and r2, and s1 to s4 are each independently an integer of 1 to 5; each instance of R² is independently hydrogen or a C₁-C₁₂ alkyl group, alkenyl group, cycloalkyl group, or cycloalkenyl group; and each instance of R⁵ is independently an alkyl group, cycloalkyl group, alkenyl group, cycloalkenyl group, aryl group, heteroalkyl group, heterocyclic group, or combination thereof optionally comprising from 1 to 8 or 2 to 8 secondary or tertiary amines or a substituent comprising a tissue-specific or cell-specific targeting moiety.

In some embodiments, the method comprises preparing a polymer comprising a structure of Formula 5 from a compound of Formula A:

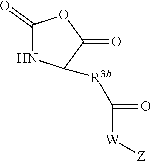

wherein,
R^{3b} is a methylene or ethylene group;
W is —NR¹⁴— or —O—, wherein R¹⁴ is hydrogen or a C₁-C₁₂ alkyl group, alkenyl group, cycloalkyl group, or cycloalkenyl group;
Z is A¹, or an alkyl group, cycloalkyl group, alkenyl group, cycloalkenyl group, aryl group, arylalkyl group, heteroalkyl group, heterocyclic group, or combination thereof optionally comprising from 1 to 8 or 2 to 8 secondary or tertiary amines or a substituent comprising a tissue-specific or cell-specific targeting moiety;
A¹ is a group of formula

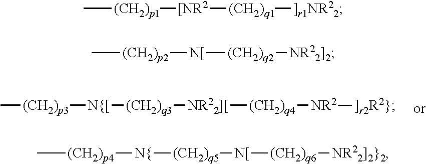

wherein p1 to p4, q1 to q6, and r1 and r2 are each independently an integer of 1 to 5; and each instance of R² is independently hydrogen or a C₁-C₁₂ alkyl group, alkenyl group, cycloalkyl group, or cycloalkenyl group.

Generally, the method comprising forming a polymer comprising a structure of Formula 5 from a compound of Formula A includes a ring-opening polymerization of the compound Formula A. The ring-opening polymerization of the compound of Formula A can be initiated by any suitable method (e.g., temperature, light, catalyst, compound, etc.). In some embodiments, the ring-opening polymerization of the compound of Formula A is initiated with an amine-containing compound.

In certain embodiments, the ring-opening polymerization of the compound of Formula A is initiated with an amine-containing compound of the formula:

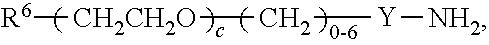

wherein,
c is an integer from 0 to 50;
Y is optionally present and is a cleavable linker; and
R⁶ is hydrogen, an amino group, an aryl group, a heterocyclic group, a C₁-C₁₂ alkyl group, alkenyl group, cycloalkyl group, or cycloalkenyl group, a C₁-C₁₂ linear or branched alkyl group optionally substituted with one or more amines; or a tissue-specific or cell-specific targeting moiety. In preferred embodiments, the amine-containing compound is

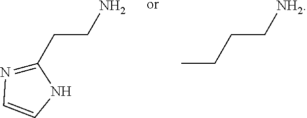

In some embodiments, in the compound of Formula A, Y is —O—, and Z is an alkyl group, cycloalkyl group, alkenyl group, cycloalkenyl group, aryl group, arylalkyl group, heteroalkyl group, heterocyclic group, or combination thereof optionally comprising from 1 to 8 or 2 to 8 secondary or tertiary amines. In some embodiments, Z is an arylalkyl group, such as a benzyl. The method of producing a polymer of Formula 5, then, further comprises, after ring opening polymerization, treating the resulting intermediate polymer with a compound of the formula NHR¹⁷-A¹, wherein A¹ is as defined with respect to Formula 1. The compound of the formula NHR¹⁷-A¹ is optionally diethyleneamine triamine. In some embodiments, the compound of formula NHR¹⁷-A¹ is not diethyleneamine triamine.

R¹⁷ can be hydrogen or a C₁-C₁₂ alkyl group, alkenyl group, cycloalkyl group, or cycloalkenyl group. In some embodiments, R¹⁷ is a C₁-C₁₂ (e.g., a C₁-C₁₀ alkyl group; a C₁-C₈ alkyl group; a C₁-C₆ alkyl group; a C₁-C₄ alkyl group, a C₁-C₃ alkyl group, or a C₁ or C₂ alkyl group) linear or branched alkyl group. In certain embodiments, R¹⁷ is methyl. In other embodiments, R¹⁷ can be hydrogen. Typically, within a given polymer, each R¹⁷ is the same (e.g., all methyl or all hydrogen); however, each R¹⁷ is independently chosen and can be the same or different.

Examples of compounds containing a leaving group suitable for use include compound of formula:

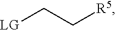

wherein LG is a leaving group (e.g., chloride atom, bromide atom, iodide atom, tosylate, triflate, mesylate, etc.) and R⁵ is as described with respect to any of Formulas 1, 1.1, 1.2, 1A, 1.1A, or 1.2A.

In some embodiments, the displacement reaction is facilitated by an acid and/or base. The acid and/or base can be any suitable acid and/or base with any suitable pKa. The acid and/or base can be an organic acid (e.g., p-toluenesulfonic acid), organic base (e.g., triethylamine), inorganic acid (e.g., titanium tetrachloride), inorganic base (e.g., potassium carbonate), or a combination thereof.

In some embodiments, the displacement reaction is facilitated by an acid. The acid can be a Brønsted acid or a Lewis acid. In embodiments where the acid is a Brønsted acid, the acid can be a weak acid (i.e., a pKa of from about 4 to about 7) or a strong acid (i.e., a pKa of from about-2 to about 4). Typically, the acid is a weak acid. In certain embodiments, the acid is a Lewis acid. For example, the acid can be bis(trifluoromethanesulfon)imide or p-toluenesulfonic acid.

In some embodiments, the displacement reaction is facilitated by a base. The base can be a weak base (i.e., a pKa of from about 7 to about 12) or a strong base (i.e., a pKa of from about 12 to about 50). Typically, the base is a weak base. For example, the base can be triethylamine, diisopropylethylamine, pyridine, N-methyl morpholine, or N,N-dimethyl-piperazine, or derivatives thereof.

In some embodiments, the displacement reaction is performed in a solvent. The solvent can be any suitable solvent, or mixture of solvents, capable of solubilizing the polymer and the compound comprising a leaving group to be reacted. For example, the solvent can include water, protic organic solvents, and/or aprotic organic solvents. An exemplary list of solvents includes water, dichloromethane, diethyl ether, dimethyl sulfoxide, acetonitrile, methanol, and ethanol.

In some embodiments, the method further comprises isolating the polymer comprising the structure of Formula 1. The polymer comprising the structure of Formula 1 can be isolated by any suitable means. For example, the polymer comprising the structure of Formula 1 can be isolated by extraction, crystallization, recrystallization, column chromatography, filtration, or any combination thereof.

### Compositions

The polymers provided herein can be used for any purpose. However, it is believed the polymers are particularly useful for delivering nucleic acids and/or polypeptides (e.g., protein) to cells. Thus, provided herein is a composition comprising a polymer as described herein and a nucleic acid and/or polypeptide (e.g., protein).

In some embodiments, the composition comprises a nucleic acid. Any nucleic acid can be used. An exemplary list of nucleic acids includes guide and/or donor nucleic acids of CRISPR systems, siRNA, microRNA, interfering RNA or RNAi, dsRNA, mRNA, DNA vector, ribozymes, antisense polynucleotides, and DNA expression cassettes encoding siRNA, microRNA, dsRNA, ribozymes or antisense nucleic acids. SiRNA comprises a double stranded structure typically containing 15-50 base pairs and preferably 19-25 base pairs and having a nucleotide sequence identical or nearly identical to an expressed target gene or RNA within the cell. An siRNA may be composed of two annealed polynucleotides or a single polynucleotide that forms a hairpin structure. MicroRNAs (miRNAs) are small noncoding polynucleotides, about 22 nucleotides long, that direct destruction or translational repression of their mRNA targets. Antisense polynucleotides comprise sequence that is complimentary to a gene or mRNA. Antisense polynucleotides include, but are not limited to: morpholinos, 2′-O-methyl polynucleotides, DNA, RNA and the like. The polynucleotide-based expression inhibitor may be polymerized in vitro, recombinant, contain chimeric sequences, or derivatives of these groups. The polynucleotide-based expression inhibitor may contain ribonucleotides, deoxyribonucleotides, synthetic nucleotides, or any suitable combination such that the target RNA and/or gene is inhibited. The polynucleotide also can be a sequence that serves as a “barcode” for purposes such as tracking delivery in vitro or in vivo.

The composition also can comprise any protein for delivery, in addition to or instead of a nucleic acid. The polypeptide can be any suitable polypeptide. For example, the polypeptide can be a zinc finger nuclease, a transcription activator-like effector nuclease (“TALEN”), a recombinase, a deaminase, an endonuclease, or a combination thereof. In some embodiments, the polypeptide is an RNA-guided endonuclease (e.g., a Cas9 polypeptide, a Cpf1 polypeptide, or variants thereof) or a DNA recombinase (e.g., a Cre polypeptide).

It is believed the polymers provided herein are particularly useful for delivering one or more components of a CRISPR system. Thus, in some embodiments, the composition comprises a guide RNA, an RNA-guided endonuclease or nucleic acid encoding same, and/or a donor nucleic acid. The composition can comprise one, two, or all three components together with the polymer described herein. Furthermore, the composition can comprise a plurality of guide RNAs, RNA-guided endonucleases or nucleic acids encoding same, and/or donor nucleic acids. For instance, multiple different guide RNAs for different target sites could be included, optionally with multiple different donor nucleic acids and even multiple different RNA guided endonucleases or nucleic acids encoding same.

Furthermore, the components of the CRISPR system can be combined with one another (when multiple components are present) and the polymer in any particular manner or order. In some embodiments, the guide RNA is complexed with an RNA endonuclease prior to combining with the polymer. In addition, or instead, the guide RNA can be linked (covalently or non-covalently) to a donor nucleic acid prior to combining with the polymer.

The compositions are not limited with respect to any particular CRISPR system (i.e., any particular guide RNA, RNA-guided endonuclease, or donor nucleic acid), many of which are known. Nevertheless, for the sake of further illustration, the components of some such systems are described below.

### Donor Nucleic Acid

The donor nucleic acid (or “donor sequence” or “donor polynucleotide” or “donor DNA”) is a nucleic acid sequence to be inserted at the cleavage site induced by an RNA-directed endonuclease (e.g., a Cas9 polypeptide or a Cpf1 polypeptide). The donor polynucleotide will contain sufficient homology to a target genomic sequence at the cleavage site, e.g. 70%, 80%, 85%, 90%, 95%, or 100% homology with the nucleotide sequences flanking the cleavage site, e.g. within about 50 bases or less of the cleavage site, e.g. within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g. 10 nucleotides or more, 50) nucleotides or more, 100 nucleotides or more, 250 nucleotides or more, 500) nucleotides or more, 1000 nucleotides or more, 5000 nucleotides or more, etc.

The donor sequence is typically not identical to the genomic sequence that it replaces. Rather, the donor sequence may contain one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some embodiments may be used for other purposes (e.g., to signify expression at the targeted genomic locus). In some embodiments, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad Sci USA 84:4959-4963; Nehls et al. (1996) Science 272:886-889. Amplification procedures such as rolling circle amplification can also be advantageously employed, as exemplified herein. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or polymer, or can be delivered by viruses (e.g., adenovirus, AAV), as described herein for nucleic acids encoding a Cas9 guide RNA and/or a Cas9 fusion polypeptide and/or donor polynucleotide.

### Guide Nucleic Acid

In some embodiments, the composition comprises guide nucleic acid. Guide nucleic acids suitable for inclusion in a composition of the present disclosure include single-molecule guide RNAs (“single-guide RNA”/“sgRNA”) and dual-molecule guide nucleic acids (“dual-guide RNA”/“dgRNA”).

A guide nucleic acid (e.g., guide RNA) suitable for inclusion in a complex of the present disclosure directs the activities of an RNA-guided endonuclease (e.g., a Cas9 or Cpf1 polypeptide) to a specific target sequence within a target nucleic acid. A guide nucleic acid (e.g., guide RNA) comprises: a first segment (also referred to herein as a “nucleic acid targeting segment”, or simply a “targeting segment”); and a second segment (also referred to herein as a “protein-binding segment”). The terms “first” and “second” do not imply the order in which the segments occur in the guide RNA. The order of the elements relative to one another depends upon the particular RNA-guided polypeptide to be used. For instance, guide RNA for Cas9 typically has the protein-binding segment located 3′ of the targeting segment, whereas guide RNA for Cpf1 typically has the protein-binding segment located 5′ of the targeting segment.

The guide RNA may be introduced into a cell in linear or circular form. If introduced in linear form, the ends of the guide RNA may be protected (e.g., from exonucleolytic degradation) by methods known to those of skill in the art. Amplification procedures such as rolling circle amplification can also be advantageously employed, as exemplified herein.

### First Segment: Targeting Segment

The first segment of a guide nucleic acid (e.g., guide RNA) includes a nucleotide sequence that is complementary to a sequence (a target site) in a target nucleic acid. In other words, the targeting segment of a guide nucleic acid (e.g., guide RNA) can interact with a target nucleic acid (e.g., an RNA, a DNA, a double-stranded DNA) in a sequence-specific manner via hybridization (i.e., base pairing). As such, the nucleotide sequence of the targeting segment may vary and can determine the location within the target nucleic acid that the guide nucleic acid (e.g., guide RNA) and the target nucleic acid will interact. The targeting segment of a guide nucleic acid (e.g., guide RNA) can be modified (e.g., by genetic engineering) to hybridize to any desired sequence (target site) within a target nucleic acid.

The targeting segment can have a length of from 12 nucleotides to 100 nucleotides. The nucleotide sequence (the targeting sequence, also referred to as a guide sequence) of the targeting segment that is complementary to a nucleotide sequence (target site) of the target nucleic acid can have a length of 12 nt or more. For example, the targeting sequence of the targeting segment that is complementary to a target site of the target nucleic acid can have a length of 12 nt or more, 15 nt or more, 17 nt or more, 18 nt or more, 19 nt or more, 20 nt or more, 25 nt or more, 30 nt or more, 35 nt or more or 40 nt.

The percent complementarity between the targeting sequence (i.e., guide sequence) of the targeting segment and the target site of the target nucleic acid can be 60% or more (e.g., 65% or more, 70% or more, 75% or more, 80% or more, 85% or more, 90% or more, 95% or more, 97% or more, 98% or more, 99% or more, or 100%). In some embodiments, the percent complementarity between the targeting sequence of the targeting segment and the target site of the target nucleic acid is 100% over the seven contiguous 5′-most nucleotides of the target site of the target nucleic acid. In some embodiments, the percent complementarity between the targeting sequence of the targeting segment and the target site of the target nucleic acid is 60% or more over 20 contiguous nucleotides. In some embodiments, the percent complementarity between the targeting sequence of the targeting segment and the target site of the target nucleic acid is 100% over the seventeen, eighteen, nineteen or twenty contiguous 5′-most nucleotides of the target site of the target nucleic acid and as low as 0% or more over the remainder. In such a case, the targeting sequence can be considered to be 17, 18, 19 or 20 nucleotides in length, respectively.

### Second Segment: Protein-Binding Segment

The protein-binding segment of a guide nucleic acid (e.g., guide RNA) interacts with (binds) an RNA-guided endonuclease. The guide nucleic acid (e.g., guide RNA) guides the bound endonuclease to a specific nucleotide sequence within target nucleic acid (the target site) via the above mentioned targeting segment/targeting sequence/guide sequence. The protein-binding segment of a guide nucleic acid (e.g., guide RNA) comprises two stretches of nucleotides that are complementary to one another. The complementary nucleotides of the protein-binding segment hybridize to form a double stranded RNA duplex (dsRNA).

### Single and Dual Guide Nucleic Acids

A dual guide nucleic acid (e.g., guide RNA) comprises two separate nucleic acid molecules. Each of the two molecules of a subject dual guide nucleic acid (e.g., guide RNA) comprises a stretch of nucleotides that are complementary to one another such that the complementary nucleotides of the two molecules hybridize to form the double stranded RNA duplex of the protein-binding segment.

In some embodiments, the duplex-forming segment of the activator is 60%, 65%, 70%, 75%, 80%, 85%, 90%, 95%, 98%, 99% or more identical or 100% identical to one of the activator (tracrRNA) molecules set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

In some embodiments, the duplex-forming segment of the targeter is 60%, 65%, 70%, 75%, 80%, 85%, 90%, 95%, 98%, 99% or more identical or 100% identical to one of the targeter (crRNA) sequences set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

A dual guide nucleic acid (e.g., guide RNA) can be designed to allow for controlled (i.e., conditional) binding of a targeter with an activator. Because a dual guide nucleic acid (e.g., guide RNA) is not functional unless both the activator and the targeter are bound in a functional complex with Cas9, a dual guide nucleic acid (e.g., guide RNA) can be inducible (e.g., drug inducible) by rendering the binding between the activator and the targeter to be inducible. As one non-limiting example, RNA aptamers can be used to regulate (i.e., control) the binding of the activator with the targeter. Accordingly, the activator and/or the targeter can include an RNA aptamer sequence.

Aptamers (e.g., RNA aptamers) are known in the art and are generally a synthetic version of a riboswitch. The terms “RNA aptamer” and “riboswitch” are used interchangeably herein to encompass both synthetic and natural nucleic acid sequences that provide for inducible regulation of the structure (and therefore the availability of specific sequences) of the nucleic acid molecule (e.g., RNA, DNA/RNA hybrid, etc.) of which they are part. RNA aptamers usually comprise a sequence that folds into a particular structure (e.g., a hairpin), which specifically binds a particular drug (e.g., a small molecule). Binding of the drug causes a structural change in the folding of the RNA, which changes a feature of the nucleic acid of which the aptamer is a part. As non-limiting examples: (i) an activator with an aptamer may not be able to bind to the cognate targeter unless the aptamer is bound by the appropriate drug; (ii) a targeter with an aptamer may not be able to bind to the cognate activator unless the aptamer is bound by the appropriate drug; and (iii) a targeter and an activator, each comprising a different aptamer that binds a different drug, may not be able to bind to each other unless both drugs are present. As illustrated by these examples, a dual guide nucleic acid (e.g., guide RNA) can be designed to be inducible.

Examples of aptamers and riboswitches can be found, for example, in: Nakamura et al., Genes Cells. 2012 May: 17 (5): 344-64; Vavalle et al., Future Cardiol. 2012 May; 8 (3): 371-82; Citartan et al., Biosens Bioelectron. 2012 Apr. 15; 34 (1): 1-11; and Liberman et al., Wiley Interdiscip Rev RNA. 2012 May-June; 3 (3): 369-84; all of which are herein incorporated by reference in their entirety.

Non-limiting examples of nucleotide sequences that can be included in a dual guide nucleic acid (e.g., guide RNA) included in International Patent Application Nos. PCT/US2016/052690 and PCT/US2017/062617, or complements thereof that can hybridize to form a protein binding segment.

A subject single guide nucleic acid (e.g., guide RNA) comprises two stretches of nucleotides (much like a “targeter” and an “activator” of a dual guide nucleic acid) that are complementary to one another, hybridize to form the double stranded RNA duplex (dsRNA duplex) of the protein-binding segment (thus resulting in a stem-loop structure), and are covalently linked by intervening nucleotides (“linkers” or “linker nucleotides”). Thus, a subject single guide nucleic acid (e.g., a single guide RNA) can comprise a targeter and an activator, each having a duplex-forming segment, where the duplex-forming segments of the targeter and the activator hybridize with one another to form a dsRNA duplex. The targeter and the activator can be covalently linked via the 3′ end of the targeter and the 5′ end of the activator. Alternatively, targeter and the activator can be covalently linked via the 5′ end of the targeter and the 3′ end of the activator.

The linker of a single guide nucleic acid can have a length of from 3 nucleotides to 100 nucleotides. In some embodiments, the linker of a single guide nucleic acid (e.g., guide RNA) is 4 nt.

An exemplary single guide nucleic acid (e.g., guide RNA) comprises two complementary stretches of nucleotides that hybridize to form a dsRNA duplex. In some embodiments, one of the two complementary stretches of nucleotides of the single guide nucleic acid (e.g., guide RNA) (or the DNA encoding the stretch) is 60%, 65%, 70%, 75%, 80%, 85%, 90%, 95%, 98%, 99% or more identical or 100% identical to one of the activator (tracrRNA) molecules set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

In some embodiments, one of the two complementary stretches of nucleotides of the single guide nucleic acid (e.g., guide RNA) (or the DNA encoding the stretch) is 60%, 65%, 70%, 75%, 80%, 85%, 90%, 95%, 98%, 99% or more identical or 100% identical to one of the targeter (crRNA) sequences set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

In some embodiments, one of the two complementary stretches of nucleotides of the single guide nucleic acid (e.g., guide RNA) (or the DNA encoding the stretch) is 60%, 65%, 70%, 75%, 80%, 85%, 90%, 95%, 98%, 99% or more identical or 100% identical to one of the targeter (crRNA) sequences or activator (tracrRNA) sequences set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

Appropriate cognate pairs of targeters and activators can be routinely determined by taking into account the species name and base-pairing (for the dsRNA duplex of the protein-binding domain) Any activator/targeter pair can be used as part of dual guide nucleic acid (e.g., guide RNA) or as part of a single guide nucleic acid (e.g., guide RNA).

In some embodiments, an activator (e.g., a trRNA, trRNA-like molecule, etc.) of a dual guide nucleic acid (e.g., guide RNA) (e.g., a dual guide RNA) or a single guide nucleic acid (e.g., guide RNA) (e.g., a single guide RNA) includes a stretch of nucleotides with 60%, 65%, 70%, 75%, 80%, 85%, 90%, 95%, 98%, 99% or more, or 100% sequence identity with an activator (tracrRNA) molecule set forth in International Patent Application Nos. PCT/US2016/052690 and PCT/US2017/062617, or a complement thereof.

In some embodiments, an activator (e.g., a trRNA, trRNA-like molecule, etc.) of a dual guide nucleic acid (e.g., a dual guide RNA) or a single guide nucleic acid (e.g., a single guide RNA) includes 30 or more nucleotides (nt) (e.g., 40 or more, 50 or more, 60 or more, 70 or more, 75 or more nt). In some embodiments, an activator (e.g., a trRNA, trRNA-like molecule, etc.) of a dual guide nucleic acid (e.g., a dual guide RNA) or a single guide nucleic acid (e.g., a single guide RNA) has a length in a range of from 30 to 200 nucleotides (nt).

The protein-binding segment can have a length of from 10 nucleotides to 100 nucleotides.

Also with regard to both a subject single guide nucleic acid (e.g., single guide RNA) and to a subject dual guide nucleic acid (e.g., dual guide RNA), the dsRNA duplex of the protein-binding segment can have a length from 6 base pairs (bp) to 50 bp. The percent complementarity between the nucleotide sequences that hybridize to form the dsRNA duplex of the protein-binding segment can be 60% or more. For example, the percent complementarity between the nucleotide sequences that hybridize to form the dsRNA duplex of the protein-binding segment can be 65% or more, 70% or more, 75% or more, 80% or more, 85% or more, 90% or more, 95% or more, 98% or more, or 99% or more (e.g., in some embodiments, there are some nucleotides that do not hybridize and therefore create a bulge within the dsRNA duplex. In some embodiments, the percent complementarity between the nucleotide sequences that hybridize to form the dsRNA duplex of the protein-binding segment is 100%.

### Hybrid Guide Nucleic Acids

In some embodiments, a guide nucleic acid is two RNA molecules (dual guide RNA). In some embodiments, a guide nucleic acid is one RNA molecule (single guide RNA). In some embodiments, a guide nucleic acid is a DNA/RNA hybrid molecule. In such embodiments, the protein-binding segment of the guide nucleic acid is RNA and forms an RNA duplex. Thus, the duplex-forming segments of the activator and the targeter is RNA. However, the targeting segment of a guide nucleic acid can be DNA. Thus, if a DNA/RNA hybrid guide nucleic acid is a dual guide nucleic acid, the “targeter” molecule and be a hybrid molecule (e.g., the targeting segment can be DNA and the duplex-forming segment can be RNA). In such embodiments, the duplex-forming segment of the “activator” molecule can be RNA (e.g., in order to form an RNA-duplex with the duplex-forming segment of the targeter molecule), while nucleotides of the “activator” molecule that are outside of the duplex-forming segment can be DNA (in which case the activator molecule is a hybrid DNA/RNA molecule) or can be RNA (in which case the activator molecule is RNA). If a DNA/RNA hybrid guide nucleic acid is a single guide nucleic acid, then the targeting segment can be DNA, the duplex-forming segments (which make up the protein-binding segment of the single guide nucleic acid) can be RNA, and nucleotides outside of the targeting and duplex-forming segments can be RNA or DNA.

A DNA/RNA hybrid guide nucleic can be useful in some embodiments, for example, when a target nucleic acid is an RNA. Cas9 normally associates with a guide RNA that hybridizes with a target DNA, thus forming a DNA-RNA duplex at the target site. Therefore, when the target nucleic acid is an RNA, it is sometimes advantageous to recapitulate a DNA-RNA duplex at the target site by using a targeting segment (of the guide nucleic acid) that is DNA instead of RNA. However, because the protein-binding segment of a guide nucleic acid is an RNA-duplex, the targeter molecule is DNA in the targeting segment and RNA in the duplex-forming segment. Hybrid guide nucleic acids can bias Cas9 binding to single stranded target nucleic acids relative to double stranded target nucleic acids.

### Exemplary Guide Nucleic Acids

Any guide nucleic acid can be used. Many different types of guide nucleic acids are known in the art. The guide nucleic selected will be appropriately paired to the particular CRISPR system being used (e.g., the particular RNA guided endonuclease being used). Thus, the guide nucleic acid can be, for instance, a guide nucleic acid corresponding to any RNA guided endonuclease described herein or known in the art. Guide nucleic acids and RNA guided endonucleases are described, for example, in International Patent Application Nos. PCT/US2016/052690 and PCT/US2017/062617

In some embodiments, a suitable guide nucleic acid includes two separate RNA polynucleotide molecules. In some embodiments, the first of the two separate RNA polynucleotide molecules (the activator) comprises a nucleotide sequence having 60% or more (e.g., 65% or more, 70% or more, 75% or more, 80% or more, 85% or more, 90% or more, 95% or more, 98% or more, 99% or more, or 100%) nucleotide sequence identity over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides) to any one of the nucleotide sequences set forth in International Patent Application Nos. PCT/US2016/052690 and PCT/US2017/062617, or a complement thereof. In some embodiments, the second of the two separate RNA polynucleotide molecules (the targeter) comprises a nucleotide sequence having 60% or more (e.g., 65% or more, 70% or more, 75% or more, 80% or more, 85% or more, 90% or more, 95% or more, 98% or more, 99% or more, or 100%) nucleotide sequence identity over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides) to any one of the nucleotide sequences set forth in International Patent Application Nos. PCT/US2016/052690 and PCT/US2017/062617, or a complement thereof.

In some embodiments, a suitable guide nucleic acid is a single RNA polynucleotide and comprises first and second nucleotide sequence having 60% or more (e.g., 65% or more, 70% or more, 75% or more, 80% or more, 85% or more, 90% or more, 95% or more, 98% or more, 99% or more, or 100%) nucleotide sequence identity over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides) to any one of the nucleotide sequences set forth in International Patent Application Nos. PCT/US2016/052690 and PCT/US2017/062617, or complements thereof.

In some embodiments, the guide RNA is a Cpf1 and/or Cas9 guide RNA. A Cpf1 and/or Cas9 guide RNA can have a total length of from 30 nucleotides (nt) to 100 nt, e.g., from 30 nt to 40 nt, from 40 nt to 45 nt, from 45 nt to 50 nt, from 50 nt to 60 nt, from 60 nt to 70 nt, from 70 nt to 80 nt, from 80 nt to 90 nt, or from 90 nt to 100 nt. In some embodiments, a Cpf1 and/or Cas9 guide RNA has a total length of 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, or 50 nt. A Cpf1 and/or Cas9 guide RNA can include a target nucleic acid-binding segment and a duplex-forming segment.

The target nucleic acid-binding segment of a Cpf1 and/or Cas9 guide RNA can have a length of from 15 nt to 30 nt, e.g., 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt. In some embodiments, the target nucleic acid-binding segment has a length of 23 nt. In some embodiments, the target nucleic acid-binding segment has a length of 24 nt. In some embodiments, the target nucleic acid-binding segment has a length of 25 nt.

The target nucleic acid-binding segment of a Cpf1 and/or Cas9 guide RNA can have 100% complementarity with a corresponding length of target nucleic acid sequence. The targeting segment can have less than 100% complementarity with a corresponding length of target nucleic acid sequence. For example, the target nucleic acid binding segment of a Cpf1 and/or Cas9 guide RNA can have 1, 2, 3, 4, or 5 nucleotides that are not complementary to the target nucleic acid sequence. For example, in some embodiments, where a target nucleic acid-binding segment has a length of 25 nucleotides, and the target nucleic acid sequence has a length of 25 nucleotides, in some embodiments, the target nucleic acid-binding segment has 100% complementarity to the target nucleic acid sequence. As another example, in some embodiments, where a target nucleic acid-binding segment has a length of 25 nucleotides, and the target nucleic acid sequence has a length of 25 nucleotides, in some embodiments, the target nucleic acid-binding segment has 1 non-complementary nucleotide and 24 complementary nucleotides with the target nucleic acid sequence.

The duplex-forming segment of a Cpf1 and/or Cas9 guide RNA can have a length of from 15 nt to 25 nt, e.g., 15 nt, 16 nt, 17 nt, 18 nt, 19 nt, 20 nt, 21 nt, 22 nt, 23 nt, 24 nt, or 25 nt.

In some embodiments, the duplex-forming segment of a Cpf1 guide RNA can comprise the nucleotide sequence 5′-AAUUUCUACUGUUGUAGAU-3′.

### Additional Elements

In some embodiments, a guide nucleic acid (e.g., guide RNA) includes an additional segment or segments (in some embodiments at the 5′ end, in some embodiments the 3′ end, in some embodiments at either the 5′ or 3′ end, in some embodiments embedded within the sequence (i.e., not at the 5′ and/or 3′ end), in some embodiments at both the 5′ end and the 3′ end, in some embodiments embedded and at the 5′ end and/or the 3′ end, etc.). For example, a suitable additional segment can include a 5′ cap (e.g., a 7-methylguanylate cap (m⁷G)); a 3′ polyadenylated tail (i.e., a 3′ poly(A) tail); a ribozyme sequence (e.g. to allow for self-cleavage of a guide nucleic acid or component of a guide nucleic acid, e.g., a targeter, an activator, etc.); a riboswitch sequence (e.g., to allow for regulated stability and/or regulated accessibility by proteins and protein complexes); a sequence that forms a dsRNA duplex (i.e., a hairpin)); a sequence that targets an RNA to a subcellular location (e.g., nucleus, mitochondria, chloroplasts, and the like); a modification or sequence that provides for tracking (e.g., a label such as a fluorescent molecule (i.e., fluorescent dye), a sequence or other moiety that facilitates fluorescent detection: a sequence or other modification that provides a binding site for proteins (e.g., proteins that act on DNA, including transcriptional activators, transcriptional repressors, DNA methyltransferases, DNA demethylases, histone acetyltransferases, histone deacetylases, proteins that bind RNA (e.g., RNA aptamers), labeled proteins, fluorescently labeled proteins, and the like); a modification or sequence that provides for increased, decreased, and/or controllable stability; and combinations thereof.

### RNA-Guided Endonuclease

In addition to, or instead of, a guide nucleic acid, the composition can comprise an RNA-guided endonuclease protein or nucleic acid (e.g., mRNA or vector) encoding same. Any RNA-guided endonuclease can be used. The selection of the RNA guided endonuclease used will depend, at least in part, to the intended end-use of the CRISPR system employed.

In some embodiments, the polypeptide is a Cas 9 polypeptide. Suitable Cas9 polypeptides for inclusion in a composition of the present disclosure include a naturally-occurring Cas9 polypeptide (e.g., naturally occurs in bacterial and/or archaeal cells), or a non-naturally-occurring Cas9 polypeptide (e.g., the Cas) polypeptide is a variant Cas) polypeptide, a chimeric polypeptide as discussed below, and the like), as described below. In some embodiments, one skilled in the art can appreciate that the Cas9 polypeptide disclosed herein can be any variant derived or isolated from any source. In other embodiments, the Cas9 peptide of the present disclosure can include one or more of the mutations described in the literature, including but not limited to the functional mutations described in: Fonfara et al. Nucleic Acids Res. 2014 February; 42 (4): 2577-90; Nishimasu H. et al. Cell. 2014 Feb. 27; 156 (5): 935-49; Jinek M. et al. Science. 2012 337:816-21; and Jinek M. et al. Science. 2014 Mar. 14; 343 (6176); see also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference. Thus, in some embodiments, the systems and methods disclosed herein can be used with the wild type Cas9 protein having double-stranded nuclease activity, Cas9 mutants that act as single stranded nickases, or other mutants with modified nuclease activity. As such, a Cas9 polypeptide that is suitable for inclusion in a composition of the present disclosure can be an enzymatically active Cas9 polypeptide, e.g., can make single- or double-stranded breaks in a target nucleic acid, or alternatively can have reduced enzymatic activity compared to a wild-type Cas9 polypeptide.

Naturally occurring Cas9 polypeptides bind a guide nucleic acid, are thereby directed to a specific sequence within a target nucleic acid (a target site), and cleave the target nucleic acid (e.g., cleave dsDNA to generate a double strand break, cleave ssDNA, cleave ssRNA, etc.). A subject Cas9 polypeptide comprises two portions, an RNA-binding portion and an activity portion. The RNA-binding portion interacts with a subject guide nucleic acid, and an activity portion exhibits site-directed enzymatic activity (e.g., nuclease activity, activity for DNA and/or RNA methylation, activity for DNA and/or RNA cleavage, activity for histone acetylation, activity for histone methylation, activity for RNA modification, activity for RNA-binding, activity for RNA splicing etc. In some embodiments the activity portion exhibits reduced nuclease activity relative to the corresponding portion of a wild type Cas9 polypeptide. In some embodiments, the activity portion is enzymatically inactive.

Assays to determine whether a protein has an RNA-binding portion that interacts with a subject guide nucleic acid can be any convenient binding assay that tests for binding between a protein and a nucleic acid. Exemplary binding assays include binding assays (e.g., gel shift assays) that involve adding a guide nucleic acid and a Cas9 polypeptide to a target nucleic acid.

Assays to determine whether a protein has an activity portion (e.g., to determine if the polypeptide has nuclease activity that cleave a target nucleic acid) can be any convenient nucleic acid cleavage assay that tests for nucleic acid cleavage. Exemplary cleavage assays that include adding a guide nucleic acid and a Cas9 polypeptide to a target nucleic acid.

In some embodiments, a suitable Cas9 polypeptide for inclusion in a composition of the present disclosure has enzymatic activity that modifies target nucleic acid (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity).

In other embodiments, a suitable Cas9 polypeptide for inclusion in a composition of the present disclosure has enzymatic activity that modifies a polypeptide (e.g., a histone) associated with target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOlating activity, ribosylation activity, deribosylation activity, myristoylation activity or demyristoylation activity).

Many Cas9 orthologues from a wide variety of species have been identified and in some embodiments, the proteins share only a few identical amino acids. All identified Cas9 orthologues have the same domain architecture with a central HNH endonuclease domain and a split RuvC/RNaseH domain. Cas9 proteins share 4 key motifs with a conserved architecture. Motifs 1, 2, and 4 are RuvC like motifs while motif 3 is an HNH-motif.

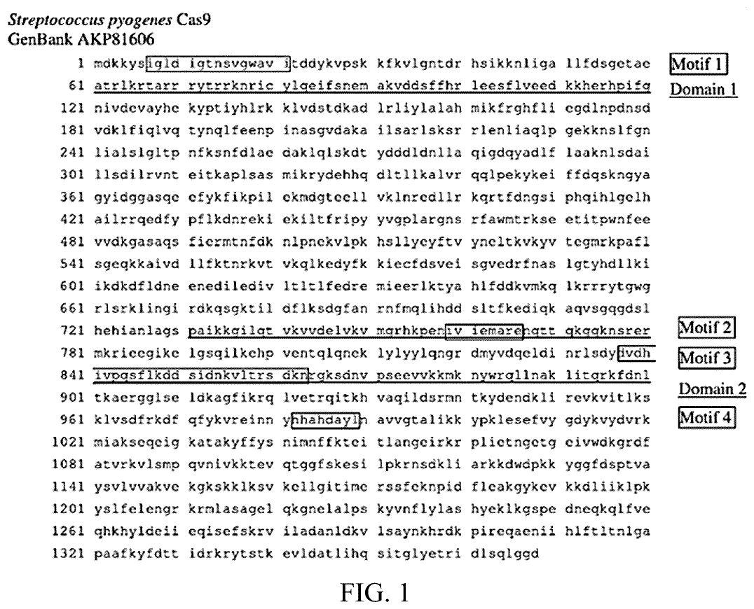

In some embodiments, a suitable Cas9 polypeptide comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity to the Cas9 amino acid sequence depicted in FIG. 1 (SEQ ID NO:1); or alternatively to motifs 1-4 of the Cas9 amino acid sequence depicted in Table 1 below (motifs 1-4 of SEQ ID NO:1 are SEQ ID NOs: 3-6, respectively, as depicted in Table 1 below); or alternatively to amino acids 7-166 or 731-1003 of the Cas9 amino acid sequence depicted in FIG. 1 (SEQ ID NO:1)

In some embodiments, a Cas9 polypeptide comprises an amino acid sequence having 75% or more, 80% or more, 85% or more, 90% or more, 95% or more, or 98%, amino acid sequence identity to the amino acid sequence depicted in FIG. 1 and set forth in SEQ ID NO: 1; and comprises amino acid substitutions of N497, R661, Q695, and Q926 relative to the amino acid sequence set forth in SEQ ID NO: 1; or comprises an amino acid substitution of K855 relative to the amino acid sequence set forth in SEQ ID NO: 1; or comprises amino acid substitutions of K810, K1003, and R1060 relative to the amino acid sequence set forth in SEQ ID NO: 1; or comprises amino acid substitutions of K848, K1003, and R1060 relative to the amino acid sequence set forth in SEQ ID NO:1.

As used herein, the term “Cas9 polypeptide” encompasses the term “variant Cas9 polypeptide”; and the term “variant Cas9 polypeptide” encompasses the term “chimeric Cas9 polypeptide.”

### Variant Cas9 Polypeptides

A suitable Cas9 polypeptides for inclusion in a composition of the present disclosure includes a variant Cas9 polypeptide. A variant Cas9 polypeptide has an amino acid sequence that is different by one amino acid (e.g., has a deletion, insertion, substitution, fusion) (i.e., different by at least one amino acid) when compared to the amino acid sequence of a wild type Cas9 polypeptide (e.g., a naturally occurring Cas9 polypeptide, as described above). In some instances, the variant Cas9 polypeptide has an amino acid change (e.g., deletion, insertion, or substitution) that reduces the nuclease activity of the Cas9 polypeptide. For example, in some instances, the variant Cas9 polypeptide has less than 50%, less than 40%, less than 30%, less than 20%, less than 10%, less than 5%, or less than 1% of the nuclease activity of the corresponding wild-type Cas9 polypeptide. In some embodiments, the variant Cas9 polypeptide has no substantial nuclease activity. When a Cas9 polypeptide is a variant Cas9 polypeptide that has no substantial nuclease activity, it can be referred to as “dCas9.”

In some embodiments, a variant Cas9 polypeptide has reduced nuclease activity. For example, a variant Cas9 polypeptide suitable for use in a binding method of the present disclosure exhibits less than about 20%, less than about 15%, less than about 10%, less than about 5%, less than about 1%, or less than about 0.1%, of the endonuclease activity of a wild-type Cas9 polypeptide, e.g., a wild-type Cas9 polypeptide comprising an amino acid sequence as depicted in FIG. 1 (SEQ ID NO:1).

In some embodiments, a variant Cas9 polypeptide can cleave the complementary strand of a target nucleic acid but has reduced ability to cleave the non-complementary strand of a double stranded target nucleic acid. For example, the variant Cas9 polypeptide can have a mutation (amino acid substitution) that reduces the function of the RuvC domain (e.g., “domain 1” of FIG. 1). As a non-limiting example, in some embodiments, a variant Cas9 polypeptide has a D10A mutation (e.g., aspartate to alanine at an amino acid position corresponding to position 10 of SEQ ID NO:1) and can therefore cleave the complementary strand of a double stranded target nucleic acid but has reduced ability to cleave the non-complementary strand of a double stranded target nucleic acid (thus resulting in a single strand break (SSB) instead of a double strand break (DSB) when the variant Cas9 polypeptide cleaves a double stranded target nucleic acid) (see, for example, Jinek et al., Science. 2012 Aug. 17; 337 (6096): 816-21).

In some embodiments, a variant Cas9 polypeptide can cleave the non-complementary strand of a double stranded target nucleic acid but has reduced ability to cleave the complementary strand of the target nucleic acid. For example, the variant Cas9 polypeptide can have a mutation (amino acid substitution) that reduces the function of the HNH domain (RuvC/HNH/RuvC domain motifs. “domain 2” of FIG. 1). As a non-limiting example, in some embodiments, the variant Cas9 polypeptide can have an H840A mutation (e.g., histidine to alanine at an amino acid position corresponding to position 840 of SEQ ID NO: 1) (FIG. 1) and can therefore cleave the non-complementary strand of the target nucleic acid but has reduced ability to cleave the complementary strand of the target nucleic acid (thus resulting in a SSB instead of a DSB when the variant Cas9 polypeptide cleaves a double stranded target nucleic acid). Such a Cas9 polypeptide has a reduced ability to cleave a target nucleic acid (e.g., a single stranded target nucleic acid) but retains the ability to bind a target nucleic acid (e.g., a single-stranded or a double-stranded target nucleic acid).

In some embodiments, a variant Cas9 polypeptide has a reduced ability to cleave both the complementary and the non-complementary strands of a double stranded target nucleic acid. As a non-limiting example, in some embodiments, the variant Cas9 polypeptide harbors both the D10A and the H840A mutations (e.g., mutations in both the RuvC domain and the HNH domain) such that the polypeptide has a reduced ability to cleave both the complementary and the non-complementary strands of a double stranded target nucleic acid. Such a Cas9 polypeptide has a reduced ability to cleave a target nucleic acid (e.g., a single-stranded target nucleic acid or a double-stranded target nucleic acid) but retains the ability to bind a target nucleic acid (e.g., a single stranded target nucleic acid or a double-stranded target nucleic acid).

As another non-limiting example, in some embodiments, the variant Cas9 polypeptide harbors W476A and W1126A mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9) polypeptide harbors P475A, W476A, N477A, D1125A. W1126A, and D1127A mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors H840A, W476A, and W1126A, mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors H840A, D10A, W476A, and W1126A, mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors, H840A, P475A, W476A, N477A, D1125A, W1126A, and D1127A mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors D10A, H840A, P475A, W476A, N477A, D1125A, W1126A, and D1127A mutations such that the polypeptide has a reduced ability to cleave a target nucleic acid. Such a Cas9 polypeptide has a reduced ability to cleave a target nucleic acid but retains the ability to bind a target nucleic acid.

Other residues can be mutated to achieve the above effects (i.e. inactivate one or the other nuclease portions). As non-limiting examples, residues D10, G12, G17, E762, H840, N854, N863, H982, H983, A984, D986, and/or A987 can be altered (i.e., substituted) (see Table 1 for more information regarding the conservation of Cas9 amino acid residues). Also, mutations other than alanine substitutions are suitable.

In some embodiments, a variant Cas9 polypeptide that has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the variant Cas9 polypeptide can still bind to target nucleic acid in a site-specific manner (because it is still guided to a target nucleic acid sequence by a guide nucleic acid) as long as it retains the ability to interact with the guide nucleic acid.

TABLE 1
Table 1 lists 4 motifs that are present
in Cas9 sequences from various species
The amino acids listed here are from
the Cas9 from S. pyogenes (SEQ ID NO: 1).

| Motif | Motif | Amino acids (residue #s) | Highly conserved |
|---|---|---|---|
| 1 | RuvC | IGLDIGTNSVGWAVI (7-21) | D10, G12, G17 |
| | | (SEQ ID NO: 3) | |
| 2 | RuvC | IVIEMARE (759-766) | E762 |
| | | (SEQ ID NO: 4) | |
| 3 | HNH-motif | DVDHIVPQSFLKDDSIDNKVLTRSDKN | H840, N854, N863 |
| | | (837-863) (SEQ ID NO: 5) | |
| 4 | RuvC | HHAHDAYL (982-989) | H982, H983, A984, |
| | | (SEQ ID NO: 6) | D986, A987 |

In addition to the above, a variant Cas9 protein can have the same parameters for sequence identity as described above for Cas9 polypeptides. Thus, in some embodiments, a suitable variant Cas9 polypeptide comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity of the Cas9 amino acid sequence depicted in FIG. 1 (SEQ ID NO:1), or alternatively to motifs 1-4 (motifs 1-4 of SEQ ID NO: 1 are SEQ ID NOs: 3-6, respectively, as depicted in Table 1); or alternatively to amino acids 7-166 or 731-1003 of the Cas9 amino acid sequence depicted in FIG. 1 (SEQ ID NO:1. Any Cas9 protein as defined above can be used as a Cas9 polypeptide, or as part of a chimeric Cas9 polypeptide, in a composition of the present disclosure, including those specifically referenced in International Patent Application Nos. PCT/US2016/052690 and PCT/US2017/062617.

In some embodiments, a suitable variant Cas9 polypeptide comprises an amino acid sequence having 60% or more, 70% or more, 75% or more, 80% or more, 85% or more, 90% or more, 95% or more, 99% or more, or 100% amino acid sequence identity to the Cas9 amino acid sequence depicted in FIG. 1 (SEQ ID NO:1). Any Cas9 protein as defined above can be used as a variant Cas9 polypeptide or as part of a chimeric variant Cas9 polypeptide in a composition of the present disclosure, including those specifically referenced in International Patent Application Nos. PCT/US2016/052690 and PCT/US2017/062617.

### Chimeric Polypeptides (Fusion Polypeptides)

In some embodiments, a variant Cas9 polypeptide is a chimeric Cas9 polypeptide (also referred to herein as a fusion polypeptide, e.g., a “Cas9 fusion polypeptide”). A Cas9 fusion polypeptide can bind and/or modify a target nucleic acid (e.g., cleave, methylate, demethylate, etc.) and/or a polypeptide associated with target nucleic acid (e.g., methylation, acetylation, etc., of, for example, a histone tail).

A Cas9 fusion polypeptide is a variant Cas9 polypeptide by virtue of differing in sequence from a wild type Cas9 polypeptide (e.g., a naturally occurring Cas9 polypeptide). A Cas9 fusion polypeptide is a Cas9 polypeptide (e.g., a wild type Cas9 polypeptide, a variant Cas9 polypeptide, a variant Cas9 polypeptide with reduced nuclease activity (as described above), and the like) fused to a covalently linked heterologous polypeptide (also referred to as a “fusion partner”). In some embodiments, a Cas9 fusion polypeptide is a variant Cas9 polypeptide with reduced nuclease activity (e.g., dCas9) fused to a covalently linked heterologous polypeptide. In some embodiments, the heterologous polypeptide exhibits (and therefore provides for) an activity (e.g., an enzymatic activity) that will also be exhibited by the Cas9 fusion polypeptide (e.g., methyltransferase activity, acetyltransferase activity, kinase activity, ubiquitinating activity, etc.). In some such embodiments, a method of binding, e.g., where the Cas9 polypeptide is a variant Cas9 polypeptide having a fusion partner (i.e., having a heterologous polypeptide) with an activity (e.g., an enzymatic activity) that modifies the target nucleic acid, the method can also be considered to be a method of modifying the target nucleic acid. In some embodiments, a method of binding a target nucleic acid (e.g., a single stranded target nucleic acid) can result in modification of the target nucleic acid. Thus, in some embodiments, a method of binding a target nucleic acid (e.g., a single stranded target nucleic acid) can be a method of modifying the target nucleic acid.

In some embodiments, the heterologous sequence provides for subcellular localization, i.e., the heterologous sequence is a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an endoplasmic reticulum (ER) retention signal, and the like). In some embodiments, a variant Cas9) does not include a NLS so that the protein is not targeted to the nucleus (which can be advantageous, e.g., when the target nucleic acid is an RNA that is present in the cytosol). In some embodiments, the heterologous sequence can provide a tag (i.e., the heterologous sequence is a detectable label) for ease of tracking and/or purification (e.g., a fluorescent protein, e.g., green fluorescent protein (GFP), YFP, RFP, CFP, mCherry, tdTomato, and the like; a histidine tag, e.g., a 6XHis tag; a hemagglutinin (HA) tag; a FLAG tag; a Myc tag; and the like). In some embodiments, the heterologous sequence can provide for increased or decreased stability (i.e., the heterologous sequence is a stability control peptide, e.g., a degron, which in some embodiments is controllable (e.g., a temperature sensitive or drug controllable degron sequence, see below). In some embodiments, the heterologous sequence can provide for increased or decreased transcription from the target nucleic acid (i.e., the heterologous sequence is a transcription modulation sequence, e.g., a transcription factor/activator or a fragment thereof, a protein or fragment thereof that recruits a transcription factor/activator, a transcription repressor or a fragment thereof, a protein or fragment thereof that recruits a transcription repressor, a small molecule/drug-responsive transcription regulator, etc.). In some embodiments, the heterologous sequence can provide a binding domain (i.e., the heterologous sequence is a protein binding sequence, e.g., to provide the ability of a Cas9 fusion polypeptide to bind to another protein of interest, e.g., a DNA or histone modifying protein, a transcription factor or transcription repressor, a recruiting protein, an RNA modification enzyme, an RNA-binding protein, a translation initiation factor, an RNA splicing factor, etc.). A heterologous nucleic acid sequence may be linked to another nucleic acid sequence (e.g., by genetic engineering) to generate a chimeric nucleotide sequence encoding a chimeric polypeptide.

A subject Cas9 fusion polypeptide (Cas9 fusion protein) can have multiple (1 or more, 2 or more, 3 or more, etc.) fusion partners in any combination of the above. As an illustrative example, a Cas9) fusion protein can have a heterologous sequence that provides an activity (e.g., for transcription modulation, target modification, modification of a protein associated with a target nucleic acid, etc.) and can also have a subcellular localization sequence. In some embodiments, such a Cas9 fusion protein might also have a tag for ease of tracking and/or purification (e.g., green fluorescent protein (GFP), YFP, RFP, CFP, mCherry, tdTomato, and the like; a histidine tag, e.g., a 6XHis tag; a hemagglutinin (HA) tag; a FLAG tag; a Myc tag; and the like). As another illustrative example, a Cas9 protein can have one or more NLSs (e.g., two or more, three or more, four or more, five or more, 1, 2, 3, 4, or 5 NLSs). In some embodiments a fusion partner (or multiple fusion partners) (e.g., an NLS, a tag, a fusion partner providing an activity, etc.) is located at or near the C-terminus of Cas9. In some embodiments a fusion partner (or multiple fusion partners) (e.g., an NLS, a tag, a fusion partner providing an activity, etc.) is located at the N-terminus of Cas9. In some embodiments a Cas9 has a fusion partner (or multiple fusion partners) (e.g., an NLS, a tag, a fusion partner providing an activity, etc.) at both the N-terminus and C-terminus.

Suitable fusion partners that provide for increased or decreased stability include, but are not limited to degron sequences. Degrons are readily understood by one of ordinary skill in the art to be amino acid sequences that control the stability of the protein of which they are part. For example, the stability of a protein comprising a degron sequence is controlled in part by the degron sequence. In some embodiments, a suitable degron is constitutive such that the degron exerts its influence on protein stability independent of experimental control (i.e., the degron is not drug inducible, temperature inducible, etc.) In some embodiments, the degron provides the variant Cas9 polypeptide with controllable stability such that the variant Cas9 polypeptide can be turned “on” (i.e., stable) or “off” (i.e., unstable, degraded) depending on the desired conditions. For example, if the degron is a temperature sensitive degron, the variant Cas9 polypeptide may be functional (i.e., “on”, stable) below a threshold temperature (e.g., 42° C., 41° C., 40° C., 39° C., 38° C., 37° C., 36° C., 35° C., 34° C., 33° C., 32° C., 31° C., 30° C., etc.) but non-functional (i.e., “off”, degraded) above the threshold temperature. As another example, if the degron is a drug inducible degron, the presence or absence of drug can switch the protein from an “off” (i.e., unstable) state to an “on” (i.e., stable) state or vice versa. An exemplary drug inducible degron is derived from the FKBP12 protein. The stability of the degron is controlled by the presence or absence of a small molecule that binds to the degron.

Examples of suitable degrons include, but are not limited to those degrons controlled by Shield-1, DHFR, auxins, and/or temperature. Non-limiting examples of suitable degrons are known in the art (e.g., Dohmen et al., Science, 1994. 263 (5151): p. 1273-1276: Heat-inducible degron: a method for constructing temperature-sensitive mutants; Schoeber et al., Am J Physiol Renal Physiol. 2009 January; 296 (1): F204-11: Conditional fast expression and function of multimeric TRPV5 channels using Shield-1; Chu et al., Bioorg Med Chem Lett. 2008 Nov. 15; 18 (22): 5941-4: Recent progress with FKBP-derived destabilizing domains; Kanemaki, Pflugers Arch. 2012 Dec. 28: Frontiers of protein expression control with conditional degrons: Yang et al., Mol Cell. 2012 Nov. 30; 48 (4): 487-8: Titivated for destruction: the methyl degron: Barbour et al., Biosci Rep. 2013 Jan. 18; 33 (1): Characterization of the bipartite degron that regulates ubiquitin-independent degradation of thymidylate synthase; and Greussing et al., J Vis Exp. 2012 Nov. 10; (69): Monitoring of ubiquitin-proteasome activity in living cells using a Degron (dgn)-destabilized green fluorescent protein (GFP)-based reporter protein: all of which are hereby incorporated in their entirety by reference).

Exemplary degron sequences have been well-characterized and tested in both cells and animals. Thus, fusing Cas9 (e.g., wild type Cas9; variant Cas9; variant Cas9 with reduced nuclease activity, e.g., dCas9; and the like) to a degron sequence produces a “tunable” and “inducible” Cas9 polypeptide. Any of the fusion partners described herein can be used in any desirable combination. As one non-limiting example to illustrate this point, a Cas9 fusion protein (i.e., a chimeric Cas9 polypeptide) can comprise a YFP sequence for detection, a degron sequence for stability, and transcription activator sequence to increase transcription of the target nucleic acid. A suitable reporter protein for use as a fusion partner for a Cas9 polypeptide (e.g., wild type Cas9, variant Cas9, variant Cas9 with reduced nuclease function, etc.), includes, but is not limited to, the following exemplary proteins (or functional fragment thereof): his3, β-galactosidase, a fluorescent protein (e.g., GFP, RFP, YFP, cherry, tomato, etc., and various derivatives thereof), luciferase, β-glucuronidase, and alkaline phosphatase. Furthermore, the number of fusion partners that can be used in a Cas9 fusion protein is unlimited. In some embodiments, a Cas9 fusion protein comprises one or more (e.g. two or more, three or more, four or more, or five or more) heterologous sequences.

Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOvlating activity, deSUMOylating activity, ribosylation activity, deribosylation activity, myristovlation activity, or demyristoylation activity, any of which can be directed at modifying nucleic acid directly (e.g., methylation of DNA or RNA) or at modifying a nucleic acid-associated polypeptide (e.g., a histone, a DNA binding protein, and RNA binding protein, and the like). Further suitable fusion partners include, but are not limited to boundary elements (e.g., CTCF), proteins and fragments thereof that provide periphery recruitment (e.g., Lamin A, Lamin B, etc.), and protein docking elements (e.g., FKBP/FRB, Pil1/Aby1, etc.).

Examples of various additional suitable fusion partners (or fragments thereof) for a subject variant Cas9 polypeptide include, but are not limited to those described in the PCT patent applications: WO2010/075303, WO2012/068627, and WO2013/155555 which are hereby incorporated by reference in their entirety.

Suitable fusion partners include, but are not limited to, a polypeptide that provides an activity that indirectly increases transcription by acting directly on the target nucleic acid or on a polypeptide (e.g., a histone, a DNA-binding protein, an RNA-binding protein, an RNA editing protein, etc.) associated with the target nucleic acid. Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristovlation activity, or demyristoylation activity.

Additional suitable fusion partners include, but are not limited to, a polypeptide that directly provides for increased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.).

Non-limiting examples of fusion partners to accomplish increased or decreased transcription include transcription activator and transcription repressor domains (e.g., the Krüppel associated box (KRAB or SKD); the Mad mSIN3 interaction domain (SID); the ERF repressor domain (ERD), etc.). In some such embodiments, a Cas9) fusion protein is targeted by the guide nucleic acid to a specific location (i.e., sequence) in the target nucleic acid and exerts locus-specific regulation such as blocking RNA polymerase binding to a promoter (which selectively inhibits transcription activator function), and/or modifying the local chromatin status (e.g., when a fusion sequence is used that modifies the target nucleic acid or modifies a polypeptide associated with the target nucleic acid). In some embodiments, the changes are transient (e.g., transcription repression or activation). In some embodiments, the changes are inheritable (e.g., when epigenetic modifications are made to the target nucleic acid or to proteins associated with the target nucleic acid, e.g., nucleosomal histones).

Non-limiting examples of fusion partners for use when targeting ssRNA target nucleic acids include (but are not limited to): splicing factors (e.g., RS domains); protein translation components (e.g., translation initiation, elongation, and/or release factors: e.g., eIF4G); RNA methylases; RNA editing enzymes (e.g., RNA deaminases, e.g., adenosine deaminase acting on RNA (ADAR), including A to I and/or C to U editing enzymes); heliembodiments; RNA-binding proteins; and the like. It is understood that a fusion partner can include the entire protein or in some embodiments can include a fragment of the protein (e.g., a functional domain).

In some embodiments, the heterologous sequence can be fused to the C-terminus of the Cas9 polypeptide. In some embodiments, the heterologous sequence can be fused to the N-terminus of the Cas9 polypeptide. In some embodiments, the heterologous sequence can be fused to an internal portion (i.e., a portion other than the N- or C-terminus) of the Cas9 polypeptide.

In addition the fusion partner of a chimeric Cas9 polypeptide can be any domain capable of interacting with ssRNA (which, for the purposes of this disclosure, includes intramolecular and/or intermolecular secondary structures, e.g., double-stranded RNA duplexes such as hairpins, stem-loops, etc.), whether transiently or irreversibly, directly or indirectly, including but not limited to an effector domain selected from the group comprising: Endonucleases (for example RNase I, the CRR22 DYW domain, Dicer, and PIN (PilT N-terminus) domains from proteins such as SMG5 and SMG6); proteins and protein domains responsible for stimulating RNA cleavage (for example CPSF, CstF, CFIm and CFIIm); Exonucleases (for example XRN-1 or Exonuclease T); Deadenylases (for example HNT3); proteins and protein domains responsible for nonsense mediated RNA decay (for example UPF1, UPF2, UPF3, UPF3b, RNP S1, Y14, DEK, REF2, and SRm160)); proteins and protein domains responsible for stabilizing RNA (for example PABP); proteins and protein domains responsible for repressing translation (for example Ago2 and Ago4); proteins and protein domains responsible for stimulating translation (for example Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (for example PAP1, GLD-2, and Star-PAP); proteins and protein domains responsible for polyuridinylation of RNA (for example CI D1 and terminal uridylate transferase); proteins and protein domains responsible for RNA localization (for example from IMP1, ZBP1, She2p, She3p, and Bicaudal-D); proteins and protein domains responsible for nuclear retention of RNA (for example Rrp6); proteins and protein domains responsible for nuclear export of RNA (for example TAP, NXF1, THO, TREX, REF, and Aly); proteins and protein domains responsible for repression of RNA splicing (for example PTB, Sam68, and hnRNP A1); proteins and protein domains responsible for stimulation of RNA splicing (for example Serine/Arginine-rich (SR) domains); proteins and protein domains responsible for reducing the efficiency of transcription (for example FUS (TLS)); and proteins and protein domains responsible for stimulating transcription (for example CDK7 and HIV Tat). Alternatively, the effector domain may be selected from the group comprising Endonucleases; proteins and protein domains capable of stimulating RNA cleavage; Exonucleases; Deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable fusion partner is a PUF RNA-binding domain, which is described in more detail in WO2012068627.

Some RNA splicing factors that can be used (in whole or as fragments thereof) as fusion partners for a Cas9 polypeptide have modular organization, with separate sequence-specific RNA binding modules and splicing effector domains. For example, members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP A1 binds to exonic splicing silencers (ESSs) through its RRM domains and inhibits exon inclusion through a C-terminal Glycine-rich domain. Some splicing factors can regulate alternative use of splice site (ss) by binding to regulatory sequences between the two alternative sites. For example, ASF/SF2 can recognize ESEs and promote the use of intron proximal sites, whereas hnRNP A1 can bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple c\hat{ω}-elements that are located in either the core exon region or the exon extension region (i.e., between the two alternative 5′ splice sites). For more examples, see WO2010075303.

In some embodiments, a Cas9 polypeptide (e.g., a wild type Cas9, a variant Cas9, a variant Cas9 with reduced nuclease activity, etc.) can be linked to a fusion partner via a peptide spacer.

In some embodiments, a Cas) polypeptide comprises a “Protein Transduction Domain” or PTD (also known as a CPP-cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD attached to another molecule facilitates entry of the molecule into the nucleus (e.g., in some embodiments, a PTD includes a nuclear localization signal (NLS)). In some embodiments, a Cas9 polypeptide comprises two or more NLSs. e.g., two or more NLSs in tandem. In some embodiments, a PTD is covalently linked to the amino terminus of a Cas9) polypeptide. In some embodiments, a PTD is covalently linked to the carboxyl terminus of a Cas9 polypeptide. In some embodiments, a PTD is covalently linked to the amino terminus and to the carboxyl terminus of a Cas9 polypeptide. In some embodiments, a PTD is covalently linked to a nucleic acid (e.g., a guide nucleic acid, a polynucleotide encoding a guide nucleic acid, a polynucleotide encoding a Cas9 polypeptide, etc.). Exemplary PTDs include but are not limited to a minimal undecapeptide protein transduction domain (corresponding to residues 47-57 of HIV-1 TAT comprising YGRKKRRQRRR; SEQ ID NO:7); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9 (6): 489-96); an Drosophila Antennapedia protein transduction domain (Noguchi et al. (2003) Diabetes 52 (7): 1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:8); Transportan GWTLNSAGYLLGKINLKALAALAKKIL (SEQ ID NO:9); KALAWEAKLAKALAKALAKHLAKALAKALKCEA (SEQ ID NO:10); and RQIKIWFQNRRMKWKK (SEQ ID NO:11). Exemplary PTDs include but are not limited to, YGRKKRRQRRR (SEQ ID NO:12), RKKRRQRRR (SEQ ID NO:13); an arginine homopolymer of from 3 arginine residues to 50 arginine residues: Exemplary PTD domain amino acid sequences include, but are not limited to, any of the following: YGRKKRRQRRR (SEQ ID NO: 14); RKKRRQRR (SEQ ID NO:15); YARAAARQARA (SEQ ID NO:16); THRLPRRRRRR (SEQ ID NO: 17); and GGRRARRRRRR (SEQ ID NO:18). In some embodiments, the PTD is an activatable CPP (ACPP) (Aguilera et al. (2009) Integr Biol (Camb) June; 1 (5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

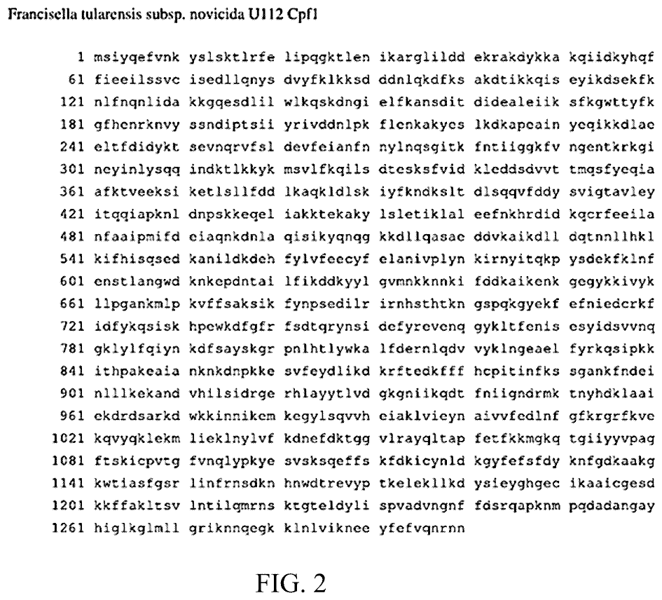

In some embodiments, the composition can comprise a Cpf1 RNA-guided endonuclease, an example of which is provided in FIG. 2, 16, or 17. Another name for the Cpf1 RNA-guided endonuclease is Cas12a. The Cpf1 CRISPR systems of the present disclosure comprise i) a single endonuclease protein, and ii) a crRNA, wherein a portion of the 3′ end of crRNA contains the guide sequence complementary to a target nucleic acid. In this system, the Cpf1 nuclease is directly recruited to the target DNA by the crRNA. In some embodiments, guide sequences for Cpf1 must be at least 12nt, 13nt, 14nt, 15nt, or 16nt in order to achieve detectable DNA cleavage, and a minimum of 14nt, 15nt, 16nt, 17nt, or 18nt to achieve efficient DNA cleavage.

The Cpf1 systems of the present disclosure differ from Cas9 in a variety of ways. First, unlike Cas9, Cpf1 does not require a separate tracrRNA for cleavage. In some embodiments, Cpf1 crRNAs can be as short as about 42-44 bases long—of which 23-25 nt is guide sequence and 19 nt is the constitutive direct repeat sequence. In contrast, the combined Cas9 tracrRNA and crRNA synthetic sequences can be about 100 bases long.

Second, Cpf1 prefers a “TTN” PAM motif that is located 5′ upstream of its target. This is in contrast to the “NGG” PAM motifs located on the 3′ of the target DNA for Cas9 systems. In some embodiments, the uracil base immediately preceding the guide sequence cannot be substituted (Zetsche, B. et al. 2015. “Cpf1 Is a Single RNA-Guided Endonuclease of a Class 2 CRISPR-Cas System” Cell 163, 759-771, which is hereby incorporated by reference in its entirety for all purposes).

Third, the cut sites for Cpf1 are staggered by about 3-5 bases, which create “sticky ends” (Kim et al., 2016. “Genome-wide analysis reveals specificities of Cpf1 endonucleases in human cells” published online Jun. 6, 2016). These sticky ends with 3-5 bp overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th base on the non-hybridized strand and the corresponding 23rd base on the complementary strand hybridized to the crRNA.

Fourth, in Cpf1 complexes, the “seed” region is located within the first 5 nt of the guide sequence. Cpf1 crRNA seed regions are highly sensitive to mutations, and even single base substitutions in this region can drastically reduce cleavage activity (see Zetsche B. et al. 2015 “Cpf1 Is a Single RNA-Guided Endonuclease of a Class 2 CRISPR-Cas System” Cell 163, 759-771). Critically, unlike the Cas9 CRISPR target, the cleavage sites and the seed region of Cpf1 systems do not overlap. Additional guidance on designing Cpf1 crRNA targeting oligos is available on (Zetsche B. et al. 2015. “Cpf1 Is a Single RNA-Guided Endonuclease of a Class 2 CRISPR-Cas System” Cell 163, 759-771).

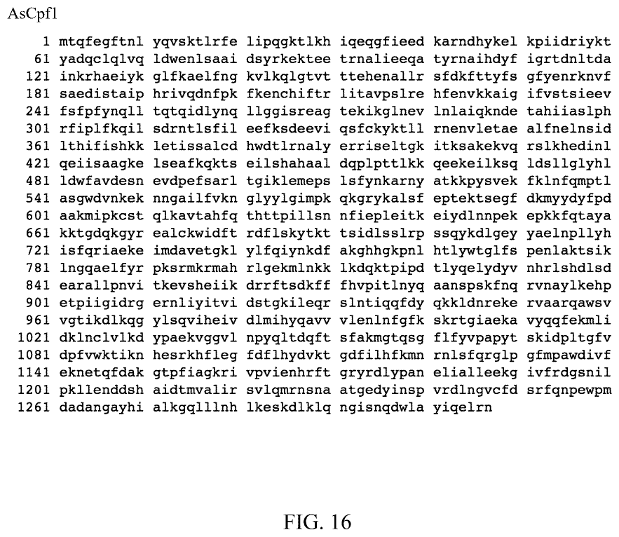

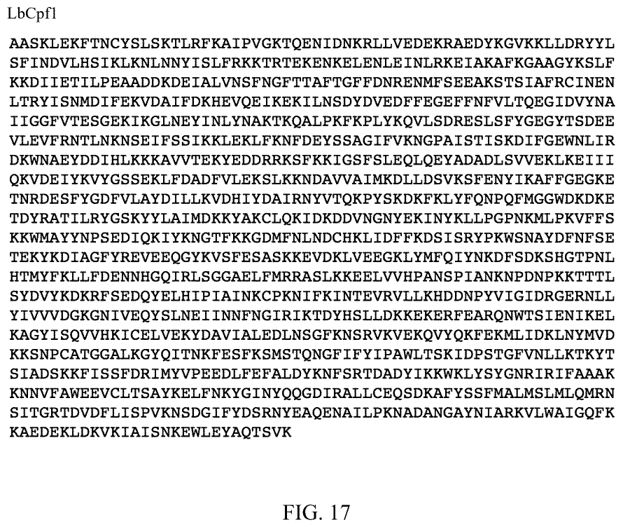

Persons skilled in the art will appreciate that the Cpf1 disclosed herein can be any variant derived or isolated from any source, many of which are known in the art. For example, in some embodiments, the Cpf1 peptide of the present disclosure can include FnCPF1 (e.g., SEQ ID NO: 2) set forth in FIG. 2, AsCpf1 (e.g., FIG. 16), LbCpf1 (e.g., FIG. 17) or any other of the many known Cpf1 proteins from various other microorganism species, and synthetic variants thereof.

In some embodiments, the composition comprises a Cpf1 polypeptide. In some embodiments, the Cpf1 polypeptide is enzymatically active, e.g., the Cpf1 polypeptide, when bound to a guide RNA, cleaves a target nucleic acid. In some embodiments, the Cpf1 polypeptide exhibits reduced enzymatic activity relative to a wild-type Cpf1 polypeptide (e.g., relative to a Cpf1 polypeptide comprising the amino acid sequence depicted in FIG. 2, 16, or 17), and retains DNA binding activity.

In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence depicted in FIG. 2, 16, or 17. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the amino acid sequence depicted in FIG. 2, 16, or 17.

In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI domain of a Cpf1 polypeptide of the amino acid sequence depicted in FIG. 2, 16, or 17. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCII domain of a Cpf1 polypeptide of the amino acid sequence depicted in FIG. 2, 16, or 17. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCIII domain of a Cpf1 polypeptide of the amino acid sequence depicted in FIG. 2, 16, or 17.

In some embodiments, the Cpf1 polypeptide exhibits reduced enzymatic activity relative to a wild-type Cpf1 polypeptide (e.g., relative to a Cpf1 polypeptide comprising the amino acid sequence depicted in FIG. 2, 16, or 17), and retains DNA binding activity. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence depicted in FIG. 2, 16, or 17; and comprises an amino acid substitution (e.g., a D→A substitution) at an amino acid residue corresponding to amino acid 917 of the amino acid sequence depicted in FIG. 2, 16, or 17. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence depicted in FIG. 2, 16, or 17; and comprises an amino acid substitution (e.g., an E→A substitution) at an amino acid residue corresponding to amino acid 1006 of the amino acid sequence depicted in FIG. 2, 16, or 17. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence depicted in FIG. 2, 16, or 17; and comprises an amino acid substitution (e.g., a D→A substitution) at an amino acid residue corresponding to amino acid 1255 of the amino acid sequence depicted in FIG. 2, 16, or 17.

In some embodiments, the Cpf1 polypeptide is a fusion polypeptide, e.g., where a Cpf1 fusion polypeptide comprises: a) a Cpf1 polypeptide; and b) a heterologous fusion partner. In some embodiments, the heterologous fusion partner is fused to the N-terminus of the Cpf1 polypeptide. In some embodiments, the heterologous fusion partner is fused to the C-terminus of the Cpf1 polypeptide. In some embodiments, the heterologous fusion partner is fused to both the N-terminus and the C-terminus of the Cpf1 polypeptide. In some embodiments, the heterologous fusion partner is inserted internally within the Cpf1 polypeptide.

Suitable heterologous fusion partners include NLS, epitope tags, fluorescent polypeptides, and the like.

### Linked Guide RNA and Donor Nucleic Acid

In one aspect, the invention provides a complex comprising a CRISPR system comprising an RNA-guided endonuclease (e.g. a Cas9 or Cpf1 polypeptide), a guide RNA and a donor polynucleotide, wherein the guide RNA and the donor polynucleotide are linked. As exemplified herein, the guide RNA and donor polynucleotide can be either covalently or non-covalently linked. In one embodiment, the guide RNA and donor polynucleotide are chemically ligated. In another embodiment, the guide RNA and donor polynucleotide are enzymatically ligated. In one embodiment, the guide RNA and donor polynucleotide hybridize to each other. In another embodiment, the guide RNA and donor polynucleotide both hybridize to a bridge sequence. Any number of such hybridization schemes are possible.

### Deaminase

In some embodiments, the complex or composition further comprises a deaminase (e.g., an adenine base editor). As used herein, the term “deaminase” or “deaminase domain” refers to an enzyme that catalyzes the removal of an amine group from a molecule, or deamination. In some embodiments, the deaminase is a cytidine deaminase, catalyzing the hydrolytic deamination of cytidine or deoxycytidine to uridine or deoxyuridine, respectively. In some embodiments, the deaminase is a cytosine deaminase, catalyzing the hydrolytic deamination of cytosine to uracil (e.g., in RNA) or thymine (e.g., in DNA).

In some embodiments, the deaminase is an adenosine deaminase, which catalyzes the hydrolytic deamination of adenine or adenosine. In some embodiments, the deaminase or deaminase domain is an adenosine deaminase, catalyzing the hydrolytic deamination of adenosine or deoxyadenosine to inosine or deoxyinosine, respectively. In some embodiments, the adenosine deaminase catalyzes the hydrolytic deamination of adenine or adenosine in deoxyribonucleic acid (DNA). The adenosine deaminases (e.g. engineered adenosine deaminases, evolved adenosine deaminases) provided herein may be from any organism, such as a bacterium. In some embodiments, the deaminase or deaminase domain is a variant of a naturally-occurring deaminase from an organism, such as a human, chimpanzee, gorilla, monkey, cow, dog, rat, or mouse.

In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as, E. coli, S aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. In some embodiments, the deaminase is APOBEC1 or a variant thereof.

The deaminase can be used in conjugation with any of the other CRISPR elements described herein (i.e., as a composition), or the deaminase can be fused to any of the other CRISPR elements (e.g., Cas9 or Cpf1) described herein (i.e., as a complex). In certain embodiments, the deaminase is fused to Cas9. Cpf1, or a variant thereof.

### Other Components

The composition can further comprise any other components typically used in nucleic acid or protein delivery formulations. For instance, the composition can further comprise lipids, lipoproteins (e.g., cholesterol and derivatives), phospholipids, polymers or other components of liposomal or micellar delivery vehicles. The composition also can comprise solvent or carrier suitable for administration to cells or hosts, such as a mammal or human.

In some embodiments, the composition comprises a second polymer that comprises polyethylene oxide (PEG). For example, the composition can comprise PEG-pAsp(DET), PEG-pAsp, derivatives of PEG-pAsp(DET), derivatives of PEG-pAsp, or a combination thereof. Without wishing to be bound by any particular theory, it is believed that these PEGylated polymers can control the size of nanoparticles and their interaction with serum proteins and target cells. The polyethylene oxide polymer can be combined with the other components in any manner and any order.

In some embodiments, the composition further comprises one or more surfactants. The surfactant can be a non-ionic surfactant and/or a zwitterionic surfactant. A list of exemplary surfactants includes, but is not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxy polyethoxyethanol) being of particular interest; (octylphenoxy) polyethoxyethanol (IGEPAL CA-6301NP-40): phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); polyoxyethylene-9-lauryl ether, and sorbitan esters (commonly known as the Spans), such as sorbitan trioleate (Span 85) and sorbitan monolaurate. In some embodiments, the surfactant is an anticoagulant (e.g., heparin or the like). In some embodiments, the composition further comprises one or more pharmaceutically acceptable carriers and/or excipients.

In some instances, a component (e.g., a nucleic acid component (e.g., a guide nucleic acid, etc.); a protein component (e.g., a Cas9 or Cpf1 polypeptide, a variant Cas9 or Cpf1 polypeptide); and the like) includes a label moiety. The terms “label”, “detectable label”, or “label moiety” as used herein refer to any moiety that provides for signal detection and may vary widely depending on the particular nature of the assay. Label moieties of interest include both directly detectable labels (direct labels) (e.g., a fluorescent label) and indirectly detectable labels (indirect labels) (e.g., a binding pair member). A fluorescent label can be any fluorescent label (e.g., a fluorescent dye (e.g., fluorescein, Texas red, rhodamine, ALEXAFLUOR® labels, and the like), a fluorescent protein (e.g., green fluorescent protein (GFP), enhanced GFP (EGFP), yellow fluorescent protein (YFP), red fluorescent protein (RFP), cyan fluorescent protein (CFP), cherry, tomato, tangerine, and any fluorescent derivative thereof), etc.). Suitable detectable (directly or indirectly) label moieties for use in the methods include any moiety that is detectable by spectroscopic, photochemical, biochemical, immunochemical, electrical, optical, chemical, or other means. For example, suitable indirect labels include biotin (a binding pair member), which can be bound by streptavidin (which can itself be directly or indirectly labeled). Labels can also include: a radiolabel (a direct label) (e.g., ³H, ¹²⁵I, ³⁵S, ¹⁴C, or ³²P); an enzyme (an indirect label) (e.g., peroxidase, alkaline phosphatase, galactosidase, luciferase, glucose oxidase, and the like); a fluorescent protein (a direct label) (e.g., green fluorescent protein, red fluorescent protein, yellow fluorescent protein, and any convenient derivatives thereof); a metal label (a direct label); a colorimetric label; a binding pair member; and the like. By “partner of a binding pair” or “binding pair member” is meant one of a first and a second moiety, wherein the first and the second moiety have a specific binding affinity for each other. Suitable binding pairs include, but are not limited to: antigen/antibodies (for example, digoxigenin/anti-digoxigenin, dinitrophenyl (DNP)/anti-DNP, dansyl-X-anti-dansyl, fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Any binding pair member can be suitable for use as an indirectly detectable label moiety.

Any given component, or combination of components can be unlabeled, or can be detectably labeled with a label moiety. In some embodiments, when two or more components are labeled, they can be labeled with label moieties that are distinguishable from one another.

### Encapsulation and Nanoparticles

In some embodiments of the composition, the polymer combines with the nucleic acid and/or polypeptide and partially or completely encapsulates the nucleic acid and/or polypeptide. The composition can, in some formulations, provide a nanoparticle comprising the polymer and nucleic acid and/or polypeptide.

In some embodiments, the composition can comprise a core nanoparticle in addition to the polymer described herein and the nucleic acid or polypeptide. Any suitable nanoparticle can be used, including metal (e.g., gold) nanoparticles or polymer nanoparticles.

The polymer described herein and the nucleic acid (e.g., guide RNA, donor polynucleotide, or both) or polypeptide can be conjugated directly or indirectly to a nanoparticle surface. For example, the polymer described herein and the nucleic acid (e.g., guide RNA, donor polynucleotide, or both) or polypeptide can be conjugated directly to the surface of a nanoparticle or indirectly through an intervening linker.

Any type of molecule can be used as a linker. For example, a linker can be an aliphatic chain including at least two carbon atoms (e.g., 3, 4, 5, 6, 7, 8, 9, 10 or more carbon atoms), and can be substituted with one or more functional groups including ketone, ether, ester, amide, alcohol, amine, urea, thiourea, sulfoxide, sulfone, sulfonamide, and disulfide functionalities. In embodiments where the nanoparticle includes gold, a linker can be any thiol-containing molecule. Reaction of a thiol group with the gold results in a covalent sulfide (—S—) bond. Linker design and synthesis are well known in the art.

In some embodiments, the nucleic acid conjugated to the nanoparticle is a linker nucleic acid that serves to non-covalently bind one or more elements described herein (e.g., a Cas9 polypeptide, and a guide RNA, a donor polynucleotide, and a Cpf1 polypeptide) to the nanoparticle-nucleic acid conjugate. For instance, the linker nucleic acid can have a sequence that hybridizes to the guide RNA or donor polynucleotide.

The nucleic acid conjugated to the nanoparticle (e.g., a colloidal metal (e.g., gold) nanoparticle: a nanoparticle comprising a biocompatible polymer) can have any suitable length. When the nucleic acid is a guide RNA or donor polynucleotide, the length will be as suitable for such molecules, as discussed herein and known in the art. If the nucleic acid is a linker nucleic acid, it can have any suitable length for a linker, for instance, a length of from 10) nucleotides (nt) to 1000 nt, e.g., from about 1 nt to about 25 nt, from about 25 nt to about 50 nt, from about 50 nt to about 100 nt, from about 100 nt to about 250 nt, from about 250) nt to about 500 nt, or from about 500 nt to about 1000 nt. In some instances, the nucleic acid conjugated to the nanoparticle (e.g., a colloidal metal (e.g., gold) nanoparticle: a nanoparticle comprising a biocompatible polymer) nanoparticle can have a length of greater than 1000 nt.

When the nucleic acid linked (e.g., covalently linked: non-covalently linked) to a nanoparticle comprises a nucleotide sequence that hybridizes to at least a portion of the guide RNA or donor polynucleotide present in a complex of the present disclosure, it has a region with sequence identity to a region of the complement of the guide RNA or donor polynucleotide sequence sufficient to facilitate hybridization. In some embodiments, a nucleic acid linked to a nanoparticle in a complex of the present disclosure has at least 50%, at least 60%, at least 70%, at least 80%, at least 90%, at least 95%, at least 98%, at least 99%, or 100%, nucleotide sequence identity to a complement of from 10 to 50 nucleotides (e.g., from 10 nucleotides (nt) to 15 nt, from 15 nt to 20 nt, from 20 nt to 25 nt, from 25 nt to 30 nt, from 30 nt to 40 nt, or from 40 nt to 50 nt) of a guide RNA or donor polynucleotide present in the complex.

In some embodiments, a nucleic acid linked (e.g., covalently linked: non-covalently linked) to a nanoparticle is a donor polynucleotide, or has the same or substantially the same nucleotide sequence as a donor polynucleotide. In some embodiments, a nucleic acid linked (e.g., covalently linked: non-covalently linked) to a nanoparticle comprises a nucleotide sequence that is complementary to a donor DNA template.

### Method of Use

The polymers provided herein can be used for any purpose, but are believed to be particularly useful for combining with and, in some embodiments, encapsulating biological molecules (e.g., nucleic acids and polypeptides) for various purposes. In one aspect, there is provided a method of encapsulating a biological molecule, such as a polypeptide or nucleic acid, by combining a polymer of the invention as described herein with the biological molecule, whereby the polymer partially or completely encapsulates the biomolecule. The biomolecule partially or completely encapsulated by the polymer is sometime referred to a as a nanoparticle.

Also provided herein is a method of delivering a nucleic acid and/or polypeptide to a cell, wherein the cell can be in vitro or in vivo. The method comprises administering a composition comprising the polymer and nucleic acid and/or polypeptide, as described herein, to the cell or to a subject containing the cell. The method can be used with respect to any type of cell or subject, but is particularly useful for mammalian cells (e.g., human cells). In some embodiments, the polymer comprises a targeting agent, such that nucleic acid and/or polypeptide is delivered predominantly or exclusively to target cells or tissues (e.g., cells or tissues of the peripheral nervous system, the central nervous system, the eye of the subject, liver, muscle, lung, bone (e.g., hematopoietic cells), or tumor cells or tissues).

When used with a composition comprising one or more components of a CRISPR system, the method may be employed to induce edit a target nucleic acid or gene. In some embodiments, a method of modifying a target nucleic acid comprises homology-directed repair (HDR). In some embodiments, use of a complex of the present disclosure to carry out HDR provides an efficiency of HDR of at least 1%, at least 2%, at least 3%, at least 4%, at least 5%, at least 10%, at least 15%, at least 20%, at least 25%, or more than 25%. In some embodiments, a method of modifying a target nucleic acid comprises non-homologous end joining (NHEJ). In some embodiments, use of a complex of the present disclosure to carry out HDR provides an efficiency of NHEJ of at least 1%, at least 2%, at least 3%, at least 4%, at least 5%, at least 10%, at least 15%, at least 20%, at least 25%, or more than 25%.

The following examples further illustrate the invention but, of course, should not be construed as in any way limiting its scope.

### Example 1

This example provides guidance for the synthesis of a polymer described herein. The synthesis includes a Michael Addition with an acrylate. An exemplary procedure is as follows.

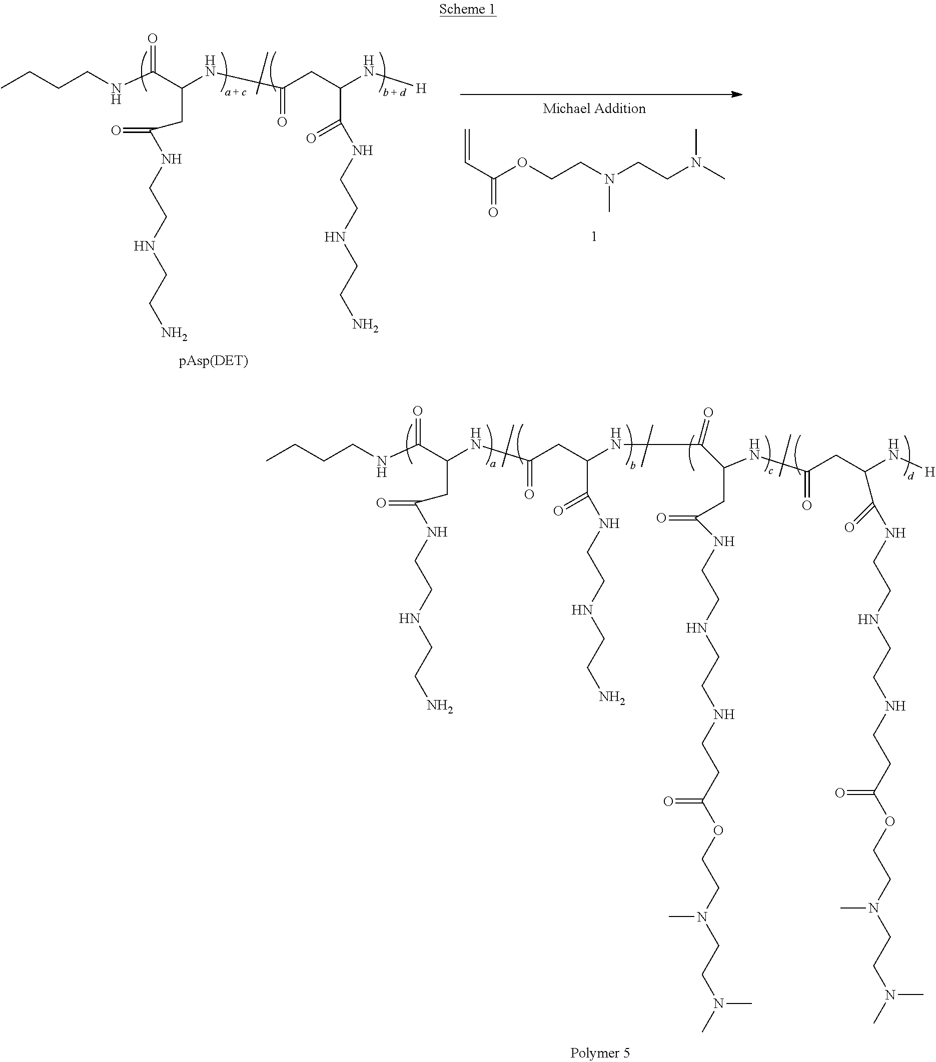

In a glass vial, pAsp(DET) (5 mg, 0.22 μmol) was suspended in dimethyl sulfoxide (“DMSO”; 700 μL). To the suspension was added 30 μL of trimethylamine (“TEA”) and the resulting suspension was stirred until all polymers completely dissolved. To the reaction mixture was added acrylate 1 (1.15 mg, 5.5 μmol) and the reaction mixture was stirred for 40 hours at room temperature. The crude product was purified by precipitation into acetonitrile and washed with acetonitrile three times to yield 4 mg of Polymer 5, wherein (a+b) is 55 and (c+d) is 25. ¹H NMR (400 MHz, D₂O): δ 4.8-4.6 (bs, 4H), 4.2 (t, 2H), 3.4-2.4 (m, 40H).

As demonstrated by Scheme 1, Michael Addition with acrylate 1 results in an amine-based bond (see, for example, Polymer 5). Thus, the original amine-functionality in pAsp(DET) remains intact.

A similar procedure can be used to prepare additional polymers of Formula 1 (e.g., Polymers 1-4 and 6-12). Nucleophilic substitution can be used to provide polymers 28 and 29.

Furthermore, the procedure can be applied to a different starting polymer, such as a polymer prepared as shown in Example 7, to provide other polymers of Formula 1 (e.g., starting with polymer 30 or 32, demethylated at the terminal nitrogen to provide a primary or secondary amine, the method can provide Polymers 13-24).

### Example 2

This example provides guidance for the synthesis of a polymer described herein. The synthesis includes a Michael Addition with an acrylate. An exemplary procedure is as follows.

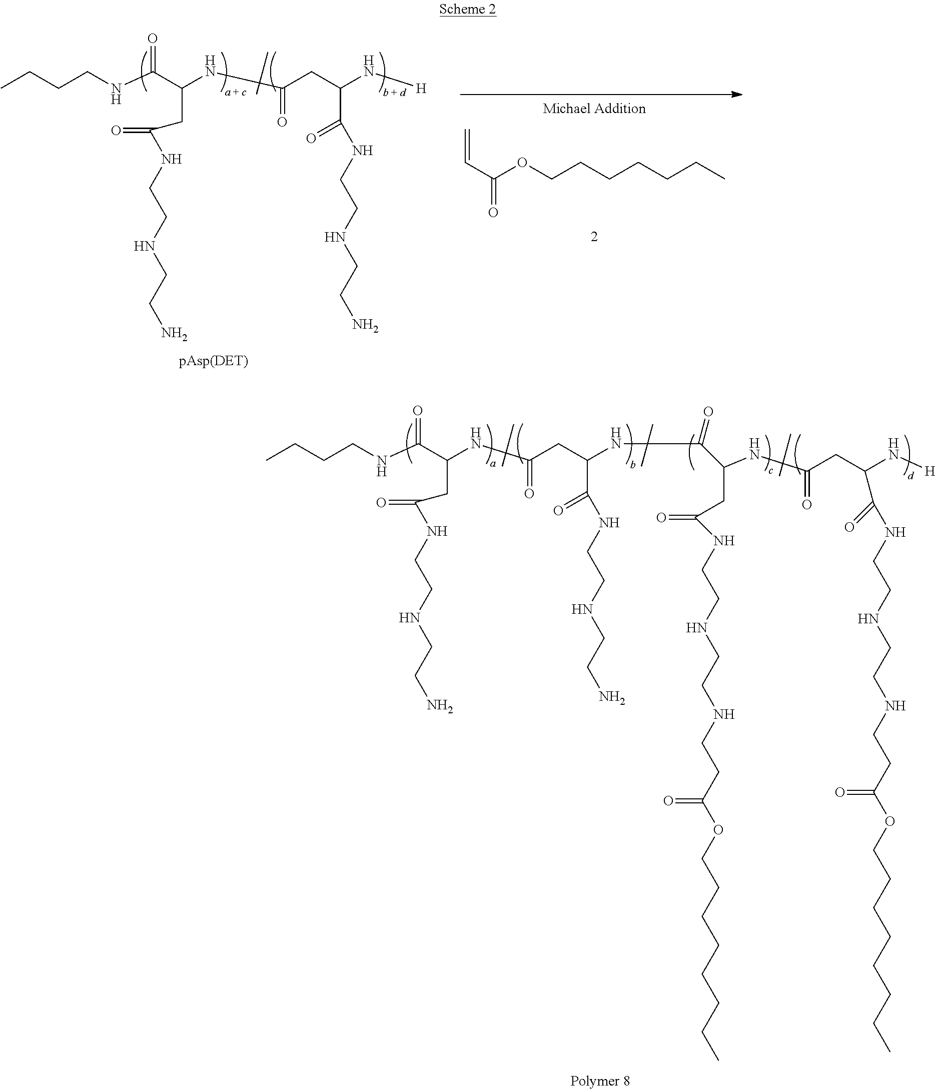

In a glass vial pASP(DET) (5 mg, 0.22 μmol) was suspended into anhydrous methanol (300 μL) and a 30 μL of triethylamine (“TEA”) was added into the suspension. The resulting solution was stirred at room temperature for 10 min to completely dissolve the polymer. The solution was diluted with 300 μL of DCM. To the reaction mixture was added acrylate 2 (1.05 mg, 5.5 μmol) in 30 μL of DCM, and the reaction mixture was stirred at room temperature for 48 hours. The crude product was purified by precipitation in large excess of diethyl ether to yield 4.3 mg of Polymer 8, wherein (a+b) is 55 and (c+d) is 25. ¹H NMR (400 MHz, D₂O): δ 4.8-4.6 (bs, 4H), 4.03 (t, 2H), 3.8-2.5 (m, 27H), 1.5 (t, 2H), 1.25 (s, 12H), 0.7 (t, 3H).

As demonstrated by Scheme 2, Michael Addition with acrylate 2 results in an amine-based bond (see, for example, Polymer 8). Thus, the original amine-functionality in pAsp(DET) remains intact.

### Example 3

This example demonstrates the ability of the polymers described herein to deliver a Cas9 ribonucleoprotein (“Cas9 RNP”) into the cell. The level of Cas9 RNP delivery was assessed using green fluorescent protein (“GFP”) inducible HEK293T (“GFP-HEK”) cells.

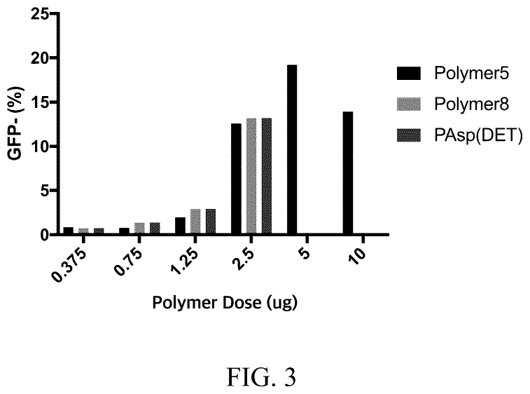

10 pmole of Cas9 RNP was mixed with (i) Polymer 5, (ii) Polymer 8, and (iii) pAsp(DET) as a control. GFP-HEK cells were treated with the resulting mixture in non-serum conditions. Polymers were added in dosages of 0.375 μg, 0.75 μg, 1.25 μg, 2.5 μg, 5 μg, and 10 μg to screen an optimal dose that gives the highest efficiency and minimal toxicity. The results are set forth in FIG. 3.

FIG. 3 shows the level of Cas9 RNP delivery as measured by the GFP(-) percent of the GFP-HEK cells treated with the three mixtures. All three polymers (i.e., Polymer 5, Polymer 8, and pAsp(DET)) showed similar levels of gene editing at polymer dosages of 0.375 μg to 2.5 μg. However, Polymer 8 and pAsp(DET) showed significant levels of cell toxicity (cell viability less than 50%) at polymer dosages of 5 μg and 10 μg. Whereas, Polymer 5 showed optimal delivery at a polymer dosage of 5 μg, as demonstrated by the level of GFP %, without significant toxicity.

### Example 4

This example demonstrates the ability of the polymers described herein to deliver Cre recombinase, which can change loxP sequences, into the cell.

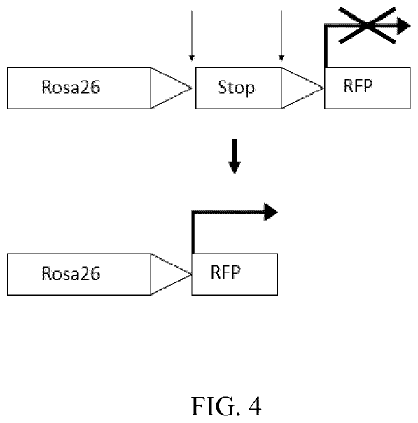

Using primary myoblasts from ai9 mice, which have a stop sequence in between loxP (see gray arrow in FIG. 4), the level of Cre recombinase delivered can be measured by RFP expression. Ai9 reporter allele contains a STOP cassette, which is flanked by the loxP sequence, thereby preventing RFP transcription. Cre recombinase removes the STOP cassette, flanked by the loxP sequence, and allows transcription of RFP.

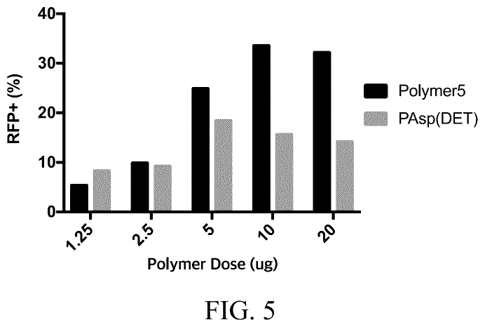

Cre recombinase (2 μg) was mixed with was mixed with (i) Polymer 5 and (ii) pAsp(DET) as a control to produce nanoparticles. Polymers were added in dosages of 1.25 μg, 2.5 μg, 5 μg, 10 μg, and 20 μg to screen an optimal dose that gives the highest efficiency and minimal toxicity. Ai9 myoblasts were treated with the resulting nanoparticles, and the RFP+ population was quantified using flow cytometry 4 days after treatment. The results are set forth in FIG. 5.

FIG. 5 shows the level of Cre recombinase delivery to primary myoblasts as measured by the amount of RFP+ expression. As shown in FIG. 5, Polymer 5 produced higher levels of RFP+ expression than the control (pAsp(DET)) at polymer dosages of 5 μg, 10 μg, and 20 μg, thereby demonstrating that Polymer 5 was more efficient at Cre recombinase delivery than pAsp(DET) at higher dosages.

### Example 5

This example demonstrates the ability of the polymers described herein to deliver Cre recombinase in vivo in mice.

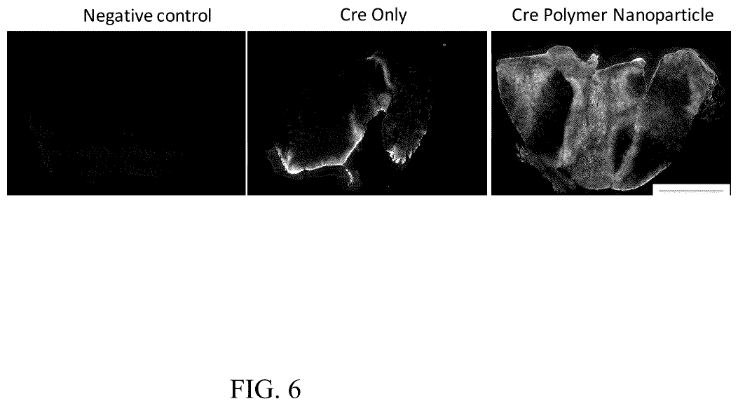

Ai9 mice were injected with (i) Cre recombinase only and (ii) a PEGylated polymer nanoparticle (i.e., a mixture of Polymer 5 and PEG-polymer) encapsulating Cre recombinase. Cre recombinase (35 μg) was delivered with a Polymer 5 and PEG-PAsp(DET) mixture (100 μg). Co-mixing of Polymer 5 and PEG-PAsp(DET) helps to control the size of polymer nanoparticles. Gastrocnemius muscle was harvested from ai9 mice two weeks after the injection. The harvested cross-sections of muscle were imaged to visualize red fluorescence protein that had been generated as a result of DNA recombination. The results are set forth in FIG. 6.

FIG. 6 shows that Cre recombinase delivery in mouse muscle was enhanced by the polymer nanoparticle. Injection with Cre recombinase only resulted in RFP expression in limited areas of muscle, as demonstrated by less red fluorescence protein visualized. Whereas, injection with Cre recombinase encapsulated by the polymer nanoparticle shows RFP expression in most of areas in gastrocnemius muscle.

A challenge of protein delivery is how widely protein can be delivered and affect large areas of tissue. The nanoparticle derived from a mixture of Polymer 5 and PEG-polymer can efficiently deliver and help distribution of Cre recombinase in ai9 mice.

### Example 6

This example demonstrates the ability of the polymers described herein to deliver Cas9 to GFP expressing neuronal cells.

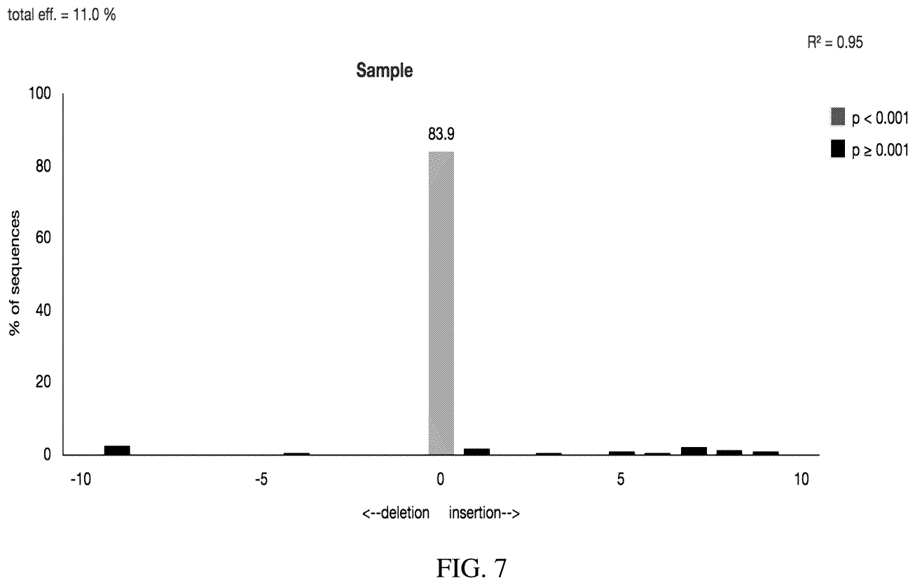

GFP expressing neuronal cells, which were differentiated from neuronal progenitor cells, were used to investigate whether Polymer 5 can deliver Cas9 RNP into neuronal cells. GFP expressing neuronal cells were treated with sgRNA and Cas9 protein using polymers described herein as a delivery method. Seven days after the treatment, genomic DNA was extracted from the cells and PCR amplification of GFP gene was conducted. TIDE analysis (TIDE software by Desktop Genetics, Netherlands Cancer Institute) was conducted to measure the frequency of indel mutation due to gene editing by Cas9. The results are set forth in FIG. 7.

As demonstrated by FIG. 7, Polymer 5 was able to deliver Cas9 RNP and induced 11% indel mutation in neuronal cells.

### Example 7

This example provides guidance for the synthesis of a polymer described herein. The synthesis includes a ring-opening polymerization, followed by further modification, to produce a polymer of Formula 4. An exemplary procedure is as follows.

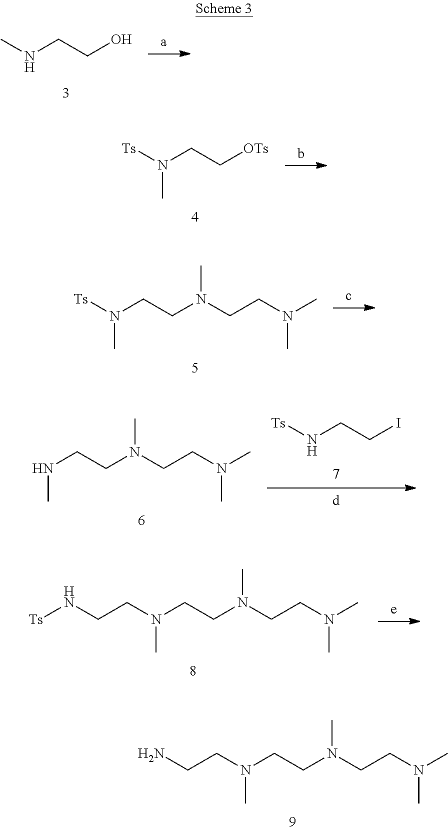

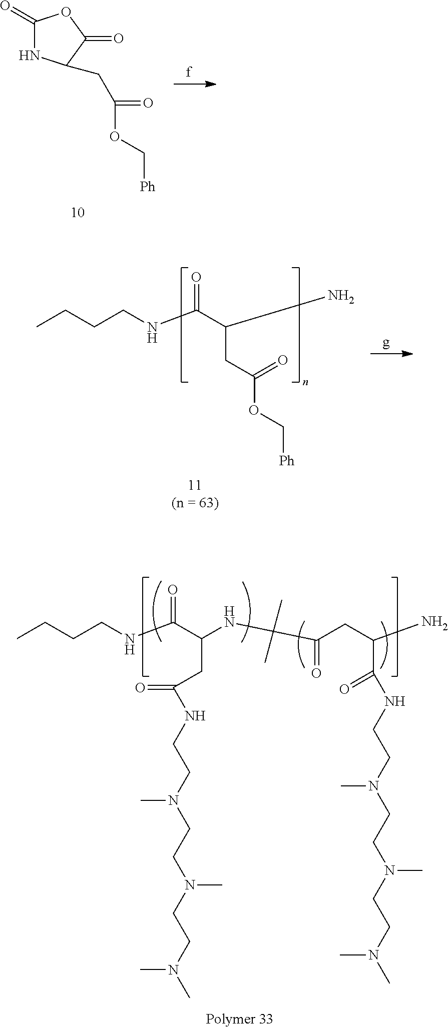

Synthesis of Polymer 33: (a) Tosyl Chloride, TEA, DCM; (b) N,N,N-trimethylethylenediamine, K₂CO₃, Acetonitrile, reflux; (c) LiAlH₄, THF; (d) 7, K₂CO₃, acetonitrile, reflux; (e) LiAlH₄, THF; (f) butylamine, DCM-DMF (9:1), 48 hours; (g) 9, NMP, 6 hours.

As demonstrated by Scheme 3, ring-opening polymerization of compound 10 results in propagation to form compound 11 with n=63, which can be further modified to form Polymer 33 upon treatment with compound 9.

A similar procedure can be used to prepare other polymers of Formula 4 (e.g., polymers 30-32). Additionally, the terminal tertiary amine on the sidechains of the polymer can be demethylated by routine techniques to provide polymers 39-42.

### Example 8

This example demonstrates the ability of the polymers described herein to deliver Cre recombinase, which can change loxP sequences, into the cell.

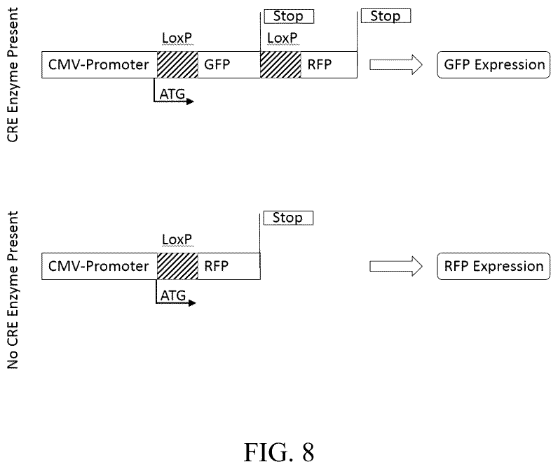

Using traffic light reporter (TLR)-HEK 293T cells, which were generated with viral transduction of traffic light reporter in HEK 293T (see FIG. 8), the level of Cre recombinase delivered can be measured by red fluorescent protein (“RFP”) expression. The traffic light reporter in HEK 293T contains a STOP cassette, and two loxP sequences that flank a GFP sequence, thereby preventing RFP transcription, and in turn expressing GFP in the absence of Cre. Cre recombinase removes the loxP sequences, and allows transcription of RFP.

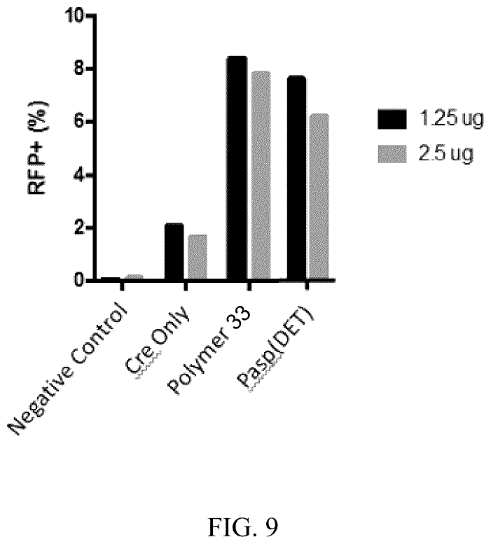

Cre recombinase (1 μg) was mixed with Polymer 33 as prepared in Example 7, or pAsp(DET) as a comparative, to produce nanoparticles. Polymers were added in dosages of 1.25 μg or 2.5 μg to screen an optimal dose that gives the highest efficiency and minimal toxicity. TLR-HEK293T cells were treated with the resulting nanoparticles, and the RFP+ population was quantified using flow cytometry 3 days after treatment. The results are set forth in FIG. 9.

FIG. 9 shows the level of Cre recombinase delivery to (TLR)-HEK 293T cells as measured by the amount of RFP+ expression. As shown in FIG. 9, Polymer 33 provided enhanced delivery over the samples containing Cre only and the comparative, pAsp(DET), at polymer dosages of 1.25 μg and 2.5 μg.

### Example 9

This example demonstrates the ability of the polymers described herein to deliver a Cas9 ribonucleoprotein (“Cas9 RNP”) into the cell. The level of Cas9 RNP delivery was assessed using green fluorescent protein (“GFP”) inducible HEK293T (“GFP-HEK”) cells.

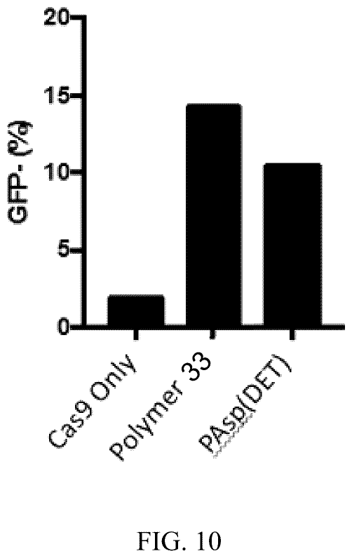

15 pmole of Cas9 RNP (sgRNA+Cas9 Protein) was mixed with Polymer 33 as prepared in Example 7, or pAsp(DET) as a comparative, to produce nanoparticles. Polymers were added in dosages 2.5 μg. GFP-HEK cells were treated with the resulting mixture in non-serum conditions. The results are set forth in FIG. 10.

FIG. 10 shows the level of Cas9 RNP delivery as measured by the GFP % of the GFP-HEK cells treated with the two mixtures. Both polymers (i.e., Polymer 33 and pAsp(DET)) showed an ability to deliver Cas9 RNP at a polymer dosage of 2.5 μg, relative to the control. However, Polymer 33 outperformed pAsp(DET) at a polymer dosage of 2.5 μg.

### Example 10

This example demonstrates the ability of the polymers described herein to deliver nucleic acids. The level of eGFP mRNA delivery to HEK 293T cells was assessed using green fluorescent protein (“GFP”).

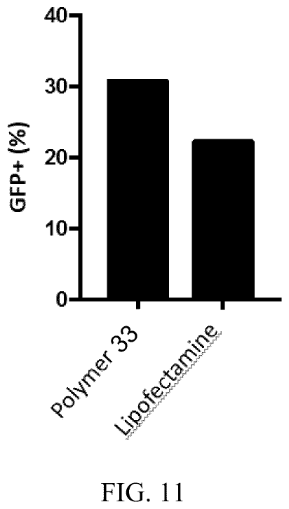

eGFP mRNA (200 ng) was mixed with Polymer 33 (600 ng) as prepared in Example 7, and incubated for 5 min. The resulting polymer nanoparticles were treated to HEK 293T cells in OptiMEM medium. After 24 hours, the cells were detached from the plate and analyzed with flow cytometry. Lipofectamine was used as a positive control for eGFP mRNA delivery. The results are set forth in FIG. 11.

FIG. 11 shows that relative to the control, Polymer 33 provides increased delivery of eGFP mRNA to HEK 293T cells.

### Example 11

This example demonstrates the effect incubation time has on the ability of the polymers described herein to deliver nucleic acids. The level of eGFP mRNA delivery to HEK 293T cells was assessed using green fluorescent protein (“GFP”).

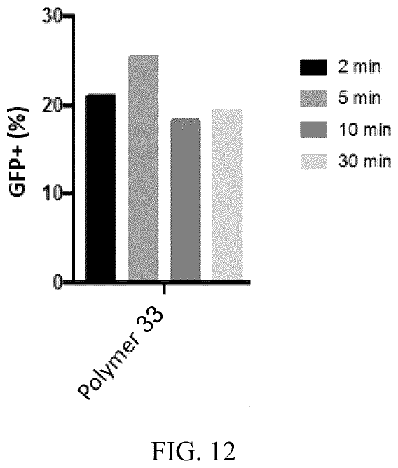

eGFP mRNA (200 ng) was mixed with Polymer 33 (1.2 μg) as prepared in Example 7, and incubated for periods of 2 min, 5 min, 10 min, and 30 min. The resulting polymer nanoparticles were treated to HEK 293T cells in OptiMEM medium. After 24 hours, the cells were detached from the plate and analyzed with flow cytometry. Lipofectamine was used as a positive control for eGFP mRNA delivery. The results are set forth in FIG. 12.

FIG. 12 shows that nanoparticles formed within 2 minutes of incubation and polymer 33 provided efficient delivery of eGFP mRNA at all incubation times.

### Example 12

This example demonstrates the ability of the polymers described herein to deliver mRNA. The level of red fluorescent protein (“RFP”) mRNA delivery to HEK 293T cells was assessed using RFP+ expression.

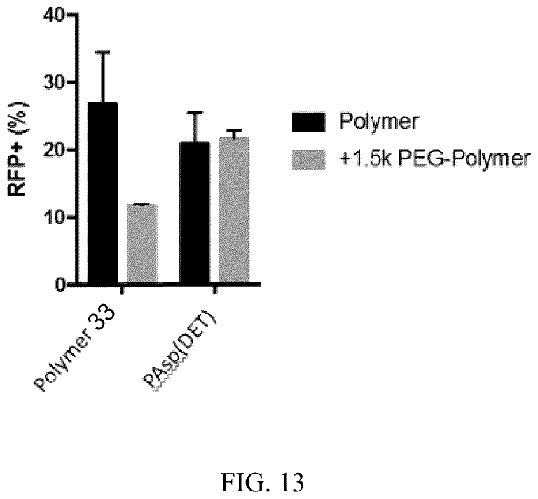

RFP mRNA (200 ng) was mixed with Polymer 33 as prepared in Example 7, or pAsp(DET) as a comparative to produce nanoparticles. Polymer 33 and pAsp(DET) were added in dosages of (i) 600 ng or (ii) 480 ng in combination with 120 ng of a 1.5 kDa PEG-PAsp(DET) polymer. The 1.5 kDa PEG-PAsp(DET) polymer can help play a role in controlling the size of the nanoparticles. TLR-HEK293T cells were treated with the resulting nanoparticles, and the RFP+ population was quantified using flow cytometry 24 hours after treatment. The results are set forth in FIG. 13.

FIG. 13 shows that relative to the comparative polymer (PAsp(DET)), Polymer 33 improved delivery of mRNAs to HEK 293T cells in the absence of the 1.5 kDa PEG-PAsp(DET) polymer. In addition, FIG. 13 shows that 600 ng of Polymer 33 provides more efficient delivery of mRNAs to HEK 293T cells than 480 ng Polymer 33 in combination with 120 ng of a 1.5 kDa PEG-PAsp(DET) polymer.

### Example 13

This example demonstrates the ability of the polymers described herein to deliver mRNAs. The level of red fluorescent protein (“RFP”) mRNA delivery to HEK 293T cells was assessed using RFP+ expression.

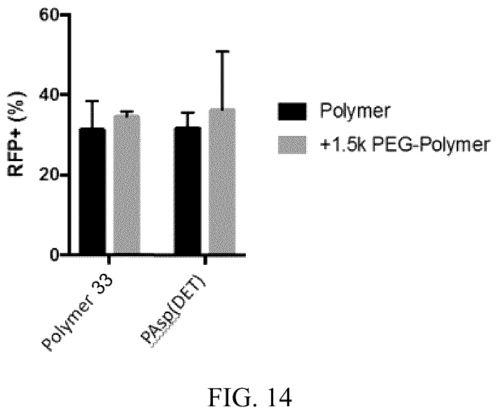

RFP mRNA (200 ng) was mixed with Polymer 33 as prepared in Example 7, or pAsp(DET) as a comparative to produce nanoparticles. Polymer 33 and pAsp(DET) were added in dosages of (i) 1 μg or (ii) 800 ng in combination with 200 ng of a 1.5 kDa PEG-PAsp(DET) polymer. The 1.5 kDa PEG-PAsp(DET) polymer can help play a role in controlling the size of the nanoparticles. TLR-HEK293T cells were treated with the resulting nanoparticles, and the RFP+ population was quantified using flow cytometry 24 hours after treatment. The results are set forth in FIG. 14.

FIG. 14 shows that relative to the comparative polymer (PAsp(DET)), Polymer 33 provides comparable delivery of mRNA to HEK 293T cells, both in the presence and in the absence of the 1.5 kDa PEG-PAsp(DET) polymer at dosages of (i) 1 μg or (ii) 800 ng in combination with 200 ng of a 1.5 kDa PEG-PAsp(DET) polymer.

### Example 14

This example demonstrates the ability of the polymers described herein to deliver a Cas9 ribonucleoprotein (“Cas9 RNP”) into the cell using different buffers. The level of Cas9 RNP delivery was assessed using green fluorescent protein (“GFP”) inducible HEK293T (“GFP-HEK”) cells.

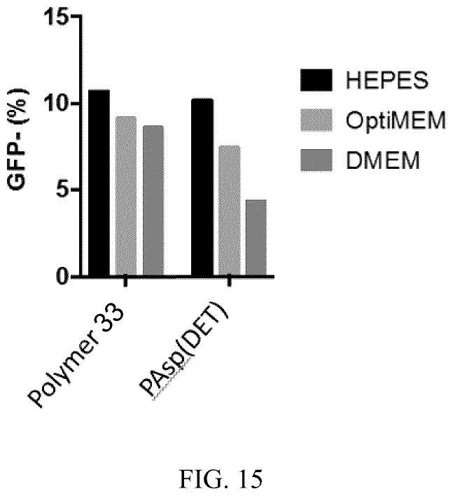

30 pmole of Cas9 RNP (sgRNA+Cas9 Protein) was mixed with 4 μg of Polymer 33 as prepared in Example 7 or 5 μg of or pAsp(DET) as a comparative, to produce nanoparticles. GFP-HEK cells (200,000 cells) were treated under three different buffer conditions, namely (i) (4-(2-hydroxyethyl)-1-piperazineethanesulfonic acid) (“HEPES”; 20 mM), (ii) Opti-MEM™ (commercially available from Thermo Fisher Scientific: Waltham, MA), or Dulbecco's Modified Eagle's Medium (“DMEM”: commercially available from Thermo Fisher Scientific: Waltham, MA). The results are set forth in FIG. 15.

FIG. 15 shows the level of Cas9 RNP delivery as measured by the GFP(-) percent of the GFP-HEK cells treated with the two mixtures (i.e., 4 μg of Polymer 33 or 5 μg of or pAsp(DET)) under the three different buffer conditions. Both polymers (i.e., Polymer 33 and pAsp(DET)) showed the same trend for the three different buffers, with HEPES outperforming Opti-MEM™, and Opti-MEM™ outperforming DMEM. In addition, Polymer 33 outperformed pAsp(DET) in all three buffers.

### Example 15

This example demonstrates the ability of the polymers described herein to stably encapsulate a nucleic acid.

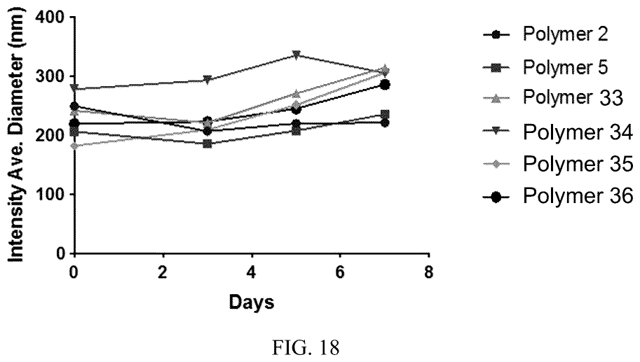

Polymers 2, 5, and 33 of the present disclosure (1 ug/uL, 10 mM HEPES) was pipet mixed with oligionucleotides (1 ug/uL, 10 mM HEPES) at a 5:1 mass ratio, and then left for 10 minutes at room temperature to form nanoparticles. They were stored at 4C when not in use. Nanoparticles from stock were diluted at ˜1:20 ratio with 10 mM HEPES and characterized by dynamic light scattering (DLS) at 0, 3, 5 and 7 days (n=1) post preparation. Intensity averaged particle size is displayed in FIG. 18.

Nanoparticle size between 150 nm to 350 nm was observed. The polymers showed minimal size changes over 7 days, indicating that the nanoparticles were stable. pAsp[DET] was run as a control (data not shown), and demonstrated a somewhat greater size change by day 7 than the other tested nanoparticles.

### Example 16

This example demonstrates the preparation of a polymer according to the disclosure

To a 100 mg (0.0074 mmol) of poly(β-benzyl 1-aspartate) (PBLA) was dissolved Into 3 mL of NMP. To this reaction mixture was added 1.5 gm of 1,4,7,10-tetramethyl-triethylenetetraamine and reaction was stirred for 16 h at RT. Crude reaction mixture was precipitated into diethyl ether and the crude product was collected via centrifuge (5000 g for 15 min). Crude product was dissolved into 3 mL of 1M HCl and dialyzed against water to achieve resulting pH=6. The resulting polymer solution was lyophilized to obtain polymer 41 as a white power.

### Example 17

This example demonstrates the use of the polymers described herein to deliver mRNA to a cell.

mRNA encoding green or red fluorescent protein was mixed with test polymer and combined with one of several different cell types as indicated in Table 2. Transfection was measured as a function of fluorescence. The results are presented in Table 2, which shows that almost all polymers produced some level of transfection in at least one cell type.

TABLE 2

| | | | Mouse Primary | | Human Neural | | | |
|---|---|---|---|---|---|---|---|---|
| | | | Myoblast | | Stem Cell | | HEK293 | Hematopoietic |
| | Hepatocyte | | No- | | No- | | No- | Stem Cell |
| Polymer | HUH-7 | HEPG2 | Serum | Serum | Serum | Serum | Serum | No-Serum |
| 1 | 0 | 0 | 0 | 0 | 0 | 1 | 0 | 0.0 |
| 2 | 0 | 0 | 0 | 0 | 0 | 1 | 0 | 4.1 |
| 3 | 0 | 0 | 0 | 1 | 12 | 1 | 0 | 0.3 |
| 4 | 0 | 0 | 0 | 0 | 0 | 1 | 1 | 0.0 |
| 5 | 0 | 0 | 3 | 1 | 19 | 3 | 12 | 0.2 |
| 6 | 0 | 0 | 0 | 0 | 0 | 0 | 0 | 0.0 |
| 7 | 9 | 0 | 0 | 1 | 7 | 3 | 6 | 1.9 |
| 8 | 7 | 0 | N/A | N/A | N/A | N/A | N/A | N/A |
| 9 | 0 | 0 | 25 | 0 | 4 | 2 | 3 | 0.9 |
| 10 | 0 | 0 | 8 | 0 | 25 | 3 | 19 | 0.7 |
| 11 | 0 | 0 | 23 | 0.5 | 18 | 4 | 25 | 0.2 |
| 12 | N/A | N/A | N/A | N/A | N/A | N/A | 9 | N/A |
| 33 | 0 | 0 | 26 | 2 | 41 | 4 | 27 | N/A |
| Lipofectamine | 84 | 43 | 39 | 52 | 49 | N/A | 26 | 0.0 |
| Mock | 0 | 0 | 0 | 0 | N/A | N/A | N/A | N/A |
| pAsp[DET] | 0 | 0 | N/A | N/A | 39 | 3 | 28 | 0.0 |

N/A = Not tested
Mock = polymer without mRNA
Mouse primary myoblast testing was performed using mRNA encoding green fluorescent protein. All other cell types were tested using mRNA encoding red fluorescent protein.

Preferred embodiments of this invention are described herein, including the best mode known to the inventors for carrying out the invention. Variations of those preferred embodiments can become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a complex” includes a plurality of such complexes and reference to “the Cas9 polypeptide” includes reference to one or more Cas9 polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

1-11. (canceled)

12. A polymer comprising a structure of Formula 4: 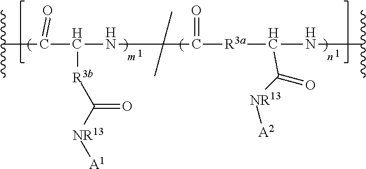
wherein,
each of m¹ and n¹ is an integer from 0 to 1000; provided that the sum of m¹+n¹ is greater than 2;
the symbol “/” indicates that the units separated thereby are linked randomly or in any order;
A¹ and A² are each independently a group of formula 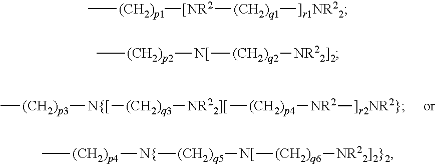
wherein p1 to p4, q1 to q6, and r1 and r2 are each independently an integer of 1 to 5;
each instance of R² is independently hydrogen or a C₁-C₁₂ alkyl group, alkenyl group, cycloalkyl group, or cycloalkenyl group, provided that A¹ and A² each comprise at least one tertiary amine;
R^{3a} and R^{3b} are each independently a methylene or ethylene group; and
each instance of R¹³ is independently hydrogen or a C₁-C₁₂ alkyl group, alkenyl group, cycloalkyl group, or cycloalkenyl group.

13. The polymer of claim 12, having the structure of Formula 4A: 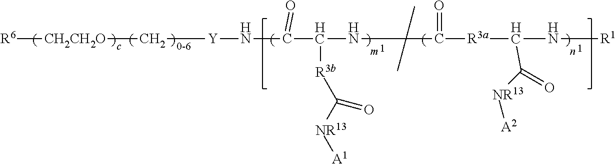
wherein,
c is an integer from 0 to 50;
Y is optionally present and is a cleavable linker;
each of m¹ and n¹ is an integer from 0 to 1000; provided that the sum of m¹+n¹ is greater than 2;
the symbol “/” indicates that the units separated thereby are linked randomly or in any order;
A¹ and A² are each independently a group of formula 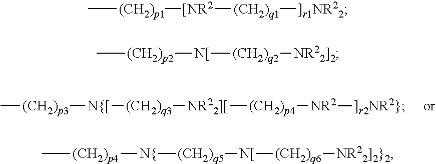
wherein p1 to p4, q1 to q6, and r1 and r2 are each independently an integer of 1 to 5; and
each instance of R² is independently a C₁-C₁₂ alkyl group, alkenyl group, cycloalkyl group, or cycloalkenyl group.
R¹ is hydrogen, an aryl group, a heterocyclic group, a C₁-C₁₂ alkyl group, alkenyl group, cycloalkyl group, or cycloalkenyl group, or a C₁-C₁₂ linear or branched alkyl group optionally substituted with one or more substituents;
each instance of R² is independently hydrogen or a C₁-C₁₂ alkyl group, alkenyl group, cycloalkyl group, or cycloalkenyl group, provided that A¹ and A² each comprise at least one tertiary amine;
R^{3a} and R^{3a} are each independently a methylene or ethylene group;
R⁶ is hydrogen, an amino group, an aryl group, a heterocyclic group, a C₁-C₁₂ alkyl group, alkenyl group, cycloalkyl group, or cycloalkenyl group, a C₁-C₁₂ linear or branched alkyl group optionally substituted with one or more amines; or a tissue-specific or cell-specific targeting moiety; and
each instance of R¹³ is independently hydrogen or a C₁-C₁₂ alkyl group, alkenyl group, cycloalkyl group, or cycloalkenyl group.

14. The polymer of claim 12, wherein each of A¹ and A² is a group of formula —(CH₂)₂—NR²—(CH₂)₂—NR²₂, wherein each instance of R² is independently a C₁-C₁₂ alkyl group, alkenyl group, cycloalkyl group, or cycloalkenyl group, with the exception that then terminal amine is a primary, secondary, or tertiary amine.

15. The polymer of claim 14, wherein each instance of R² is methyl.

16. The polymer of claim 13, wherein the tissue-specific or cell-specific targeting moiety is: 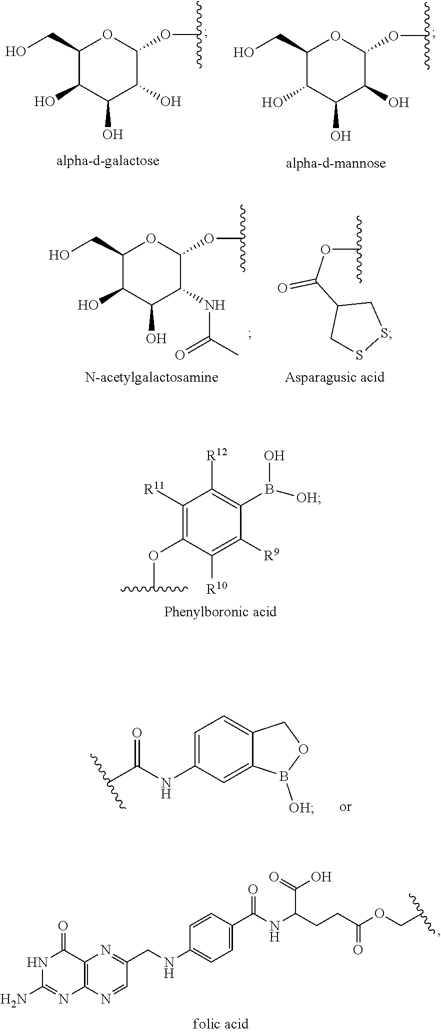
wherein each of R⁹, R¹⁰, R¹¹, and R¹² is independently hydrogen, halogen, C₁-C₄ alkyl, or C₁-C₄ alkoxy, optionally substituted with one or more amino groups.

17. The polymer of claim 12, wherein the polymer is a cationic polymer.

18. The polymer of claim 12 having the formula: 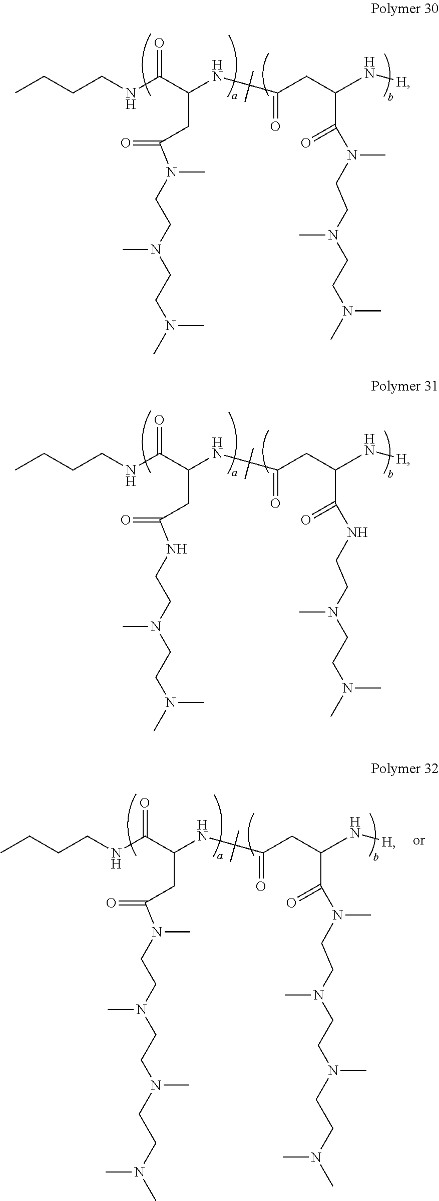 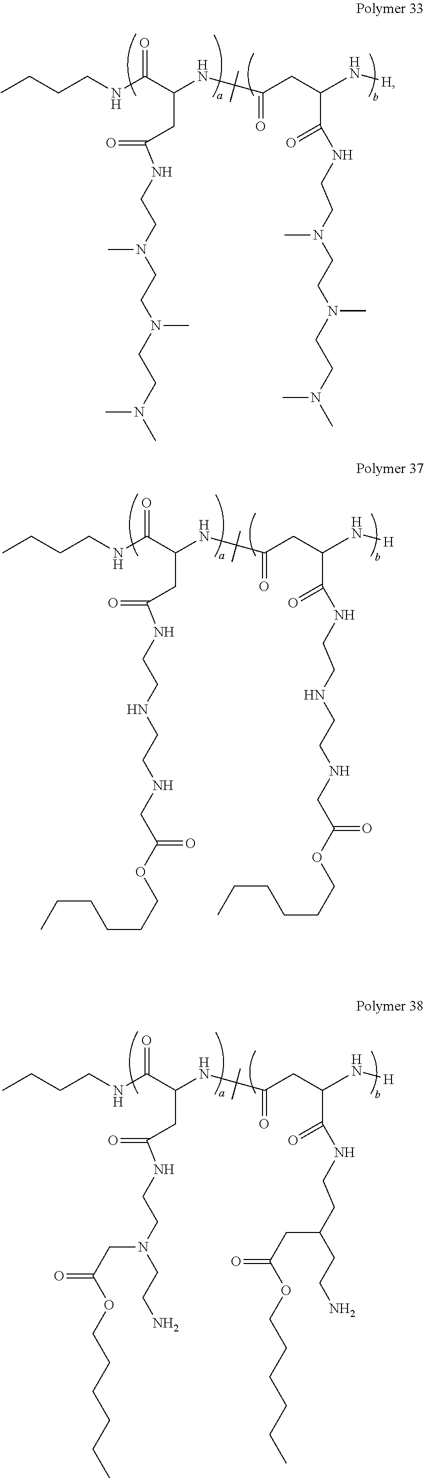 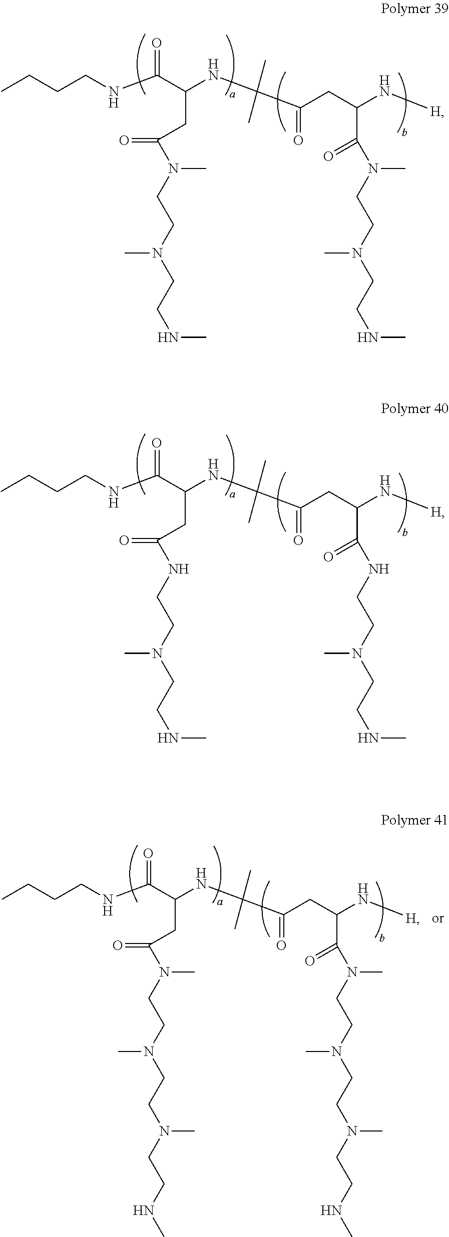 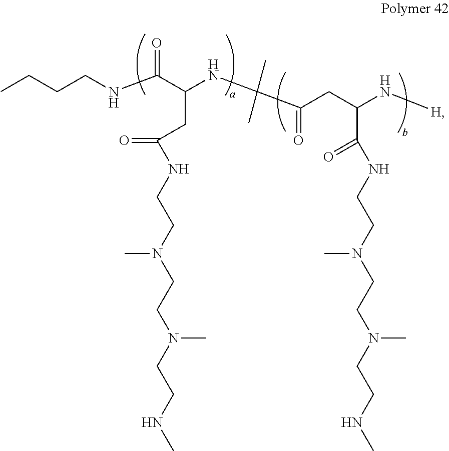
wherein (a+b) is from about 5 to about 160.

19. A method of preparing a polymer, the method comprising modifying at least a portion of groups A¹ and/or A² of a polymer comprising a structure of Formula 3: 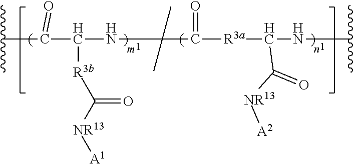
wherein:
each of m¹ and n¹ is an integer from 0 to 1000, provided that m¹+n¹ is greater than 5;
the symbol “/” indicates that the units separated thereby are linked randomly or in any order;
R^{3a} and R^{3b} are each independently a methylene or ethylene group;
each instance of R¹³ is independently hydrogen or a C₁-C₁₂ alkyl group, alkenyl group, cycloalkyl group, or cycloalkenyl group;
A¹ and A² are each independently a group of formula 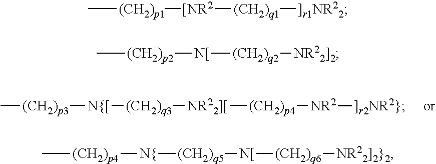
wherein p1 to p4, q1 to q6, and r1 and r2 are each independently an integer of 1 to 5; and each instance of R² is independently hydrogen or a C₁-C₁₂ alkyl group, alkenyl group, cycloalkyl group, or cycloalkenyl group,
to produce a polymer comprising a structure of Formula 1: 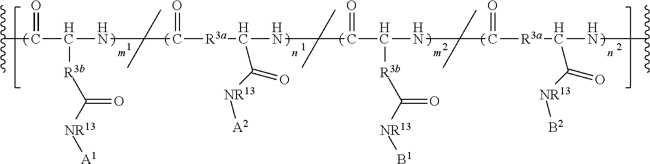
wherein:
each of m¹ and n¹ is an integer from 0 to 1000;
each of m² and n² is an integer from 0 to 1000, provided that the sum of m²+n² is greater than 5;
the symbol “/” indicates that the units separated thereby are linked randomly or in any order;
R^{3a} and R^{3b} are each independently a methylene or ethylene group;
each instance of R¹³ is independently hydrogen or a C₁-C₁₂ alkyl group, alkenyl group, cycloalkyl group, or cycloalkenyl group;
A¹ and A² are each independently a group of formula 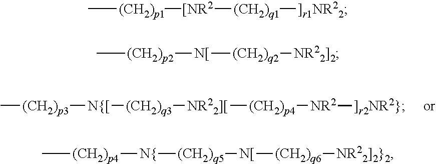
B¹ and B² are each independently 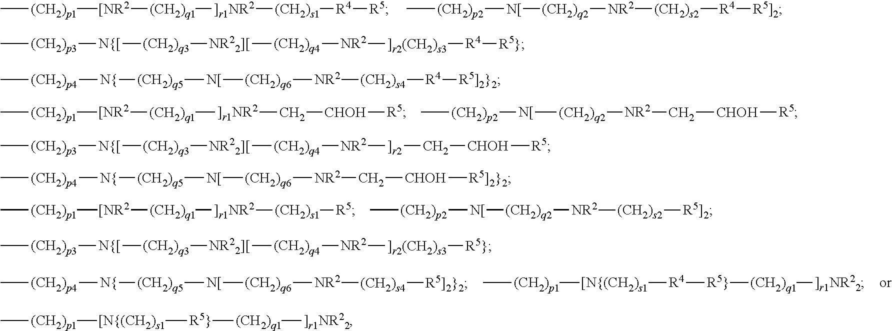
wherein p1 to p4, q1 to q6, r1 and r2, and s1 to s4 are each independently an integer of 1 to 5;
each instance of R² is independently hydrogen or a C₁-C₁₂ alkyl group, alkenyl group, cycloalkyl group, or cycloalkenyl group; each instance of R⁴ is independently —C(O)O—, —C(O)NH—, or —S(O)(O)—; and each instance of R⁵ is independently an alkyl group, cycloalkyl group, alkenyl group, cycloalkenyl group, aryl group, heteroalkyl group, heterocyclic group, or combination thereof optionally comprising from 1 to 8 secondary or tertiary amines or a substituent comprising a tissue-specific or cell-specific targeting moiety;
or
the method comprising a ring-opening polymerization of a compound Formula A by contacting an amine containing compound with a compound of Formula A, wherein the compound of Formula 5 has the structure: 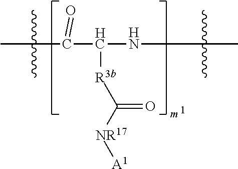
wherein,
m¹ is an integer from 1 to 2000;
R^{3b} is a methylene or ethylene group;
R¹⁷ is hydrogen or a C₁-C₁₂ alkyl group, alkenyl group, cycloalkyl group, or cycloalkenyl group;
A¹ is a group of formula 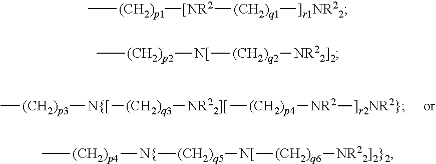
wherein p1 to p4, q1 to q6, and r1 and r2 are each independently an integer of 1 to 5; and each instance of R² is independently hydrogen or a C₁-C₁₂ alkyl group, alkenyl group, cycloalkyl group, or cycloalkenyl group,
and the compound of Formula A has the structure: 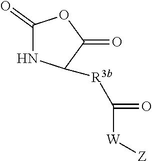
wherein,
R^{3b} is a methylene or ethylene group;
W is —NR¹⁴— or —O—, wherein R¹⁴ is hydrogen or a C₁-C₁₂ alkyl group, alkenyl group, cycloalkyl group, or cycloalkenyl group;
Z is A¹, or an alkyl group, cycloalkyl group, alkenyl group, cycloalkenyl group, aryl group, arylalkyl group, heteroalkyl group, heterocyclic group, or combination thereof optionally comprising from 1 to 8 secondary or tertiary amines or a substituent comprising a tissue-specific or cell-specific targeting moiety;
A¹ is a group of formula 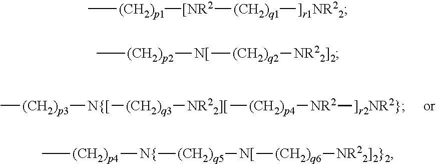
wherein p1 to p4, q1 to q6, and r1 and r2 are each independently an integer of 1 to 5; and
each instance of R² is independently hydrogen or a C₁-C₁₂ alkyl group, alkenyl group, cycloalkyl group, or cycloalkenyl group.

20-26. (canceled)

27. A composition comprising a polymer of claim 1 and a nucleic acid and/or polypeptide.

28. The composition of claim 27, wherein the composition comprises a guide nucleic acid and/or donor nucleic acid, an siRNA, or an mRNA.

29. (canceled)

30. The composition of claim 27, wherein the composition comprises an RNA-guided endonuclease or nucleic acid encoding same.

31. The composition of claim 30, wherein the RNA-guided endonuclease is Cas9, Cpf1, or a combination thereof.

32. The composition of claim 27, wherein the composition comprises a DNA recombinase, zinc finger nuclease, or transcription activator-like nuclease.

33.-35. (canceled)

36. The composition of claim 27, wherein the composition comprises a nanoparticle comprising the polymer of any of claims 1-9 and the nucleic acid or polypeptide.

37. The composition of claim 27, wherein the composition comprises a second polymer that comprises polyethylene oxide.

38. A method of delivering a nucleic acid and/or polypeptide to a cell, the method comprising administering the composition of claim 27.

39. The method of claim 38, wherein the cell is in a subject and the composition is administered to the subject.

40. The method of claim 38, wherein the polymer comprises a tissue-specific targeting moiety that localizes the polymer to tissues of the peripheral nervous system, the central nervous system, liver, muscle, lung, bone, or the eye of the subject.

41. (canceled)

42. (canceled)

43. The method of claim 38, wherein the cell is in a host, optionally a human, and the composition is delivered to the cell by administering the composition to the host.

44.-50. (canceled)

44. The polymer of claim 12, wherein each instance of R² is ethyl or methyl, with the exception that the terminal amine can be a primary, secondary, or tertiary amine, preferably a secondary or tertiary amine.
